(19) United States
(12) Patent Application Publication
(10) Pub. No.: US 2025/0376432 A1
(43) Pub. Date: Dec. 11, 2025

(54) PROCESS FOR PREPARING MODULATORS OF EUKARYOTIC INITIATION FACTOR 2B

(71) Applicant: Denali Therapeutics Inc., South San Francisco, CA (US)

(72) Inventors: Katrien Brak, Belmont, CA (US); Christopher R.H. Hale, New Brighton, MN (US); Anantha Sudhakar, Fremont, CA (US)

(21) Appl. No.: 18/877,592

(22) PCT Filed: Jun. 22, 2023
(86) PCT No.: PCT/US2023/026021

§ 371 Date: Dec. 20, 2024

Related U.S. Application Data
(60) Provisional application No. 63/355,072, filed on Jun. 23, 2022.

(51) Int. Cl. C07C 62/08; C07C 241/02; C07C 271/24; C07C 329/06; C07D 265/30; C07D 271/10

(52) U.S. Cl. CPC C07C 62/08; C07C 241/02; C07C 271/24; C07C 329/06; C07D 265/30; C07D 271/10; C07B 2200/13

(57) ABSTRACT

The present disclosure relates generally to methods for the preparation of Compound (I-1): I-1 or a stereoisomer or mixture of stereoisomers thereof, or salt of each thereof, as well as compounds and salts which are useful in the synthesis thereof.

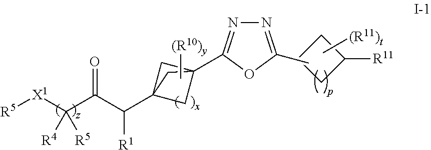

### CROSS-REFERENCE TO RELATED APPLICATIONS

This application claims the benefit under 35 U.S.C. § 119(e) of U.S. Provisional Application Ser. No. 63/355,072 filed Jun. 23, 2022, the contents of which are hereby incorporated by reference in its entirety.

### FIELD

The present application relates to processes for synthesis of small molecule modulators of eukaryotic initiation factor 2B, useful as therapeutic agents, novel intermediates, and methods for synthesizing the same.

### DESCRIPTION

The multi-subunit protein complexes eukaryotic initiation factor 2B (eIF2B) and eukaryotic initiation factor 2 (eIF2) are required for protein synthesis initiation and regulation in eukaryotic cells. The interaction between eIF2B and eIF2 plays an important role in the integrated stress response (ISR) pathway. Activation of this pathway leads in part to ATF4 (Activating Transcription Factor 4) expression and stress granule formation. Aberrant ISR activation is found in multiple neurodegenerative diseases, with a strong functional link to pathology characterized by the RNA-binding/stress-granule protein TAR DNA binding protein (TARDBP), also known as TDP43. Impairment of eIF2B activity is correlated to activation of the ISR pathway that is implicated in a variety neurodegenerative diseases including Parkinson's disease, amyotrophic lateral sclerosis (ALS), Alzheimer's disease and frontotemporal dementia, and vanishing white matter disease. Activation of eIF2B, on the other hand, inhibits the ISR and ISR dependent stress granule formation and is found to be neuroprotective in these disease models. Small molecule modulators of eIF2B are desirable to treat these conditions.

Improved methods for synthesizing small molecule modulators of eukaryotic initiation factor 2B are disclosed herein. In certain embodiments, provided herein are improved processes for preparing a compound of Formula I-1:

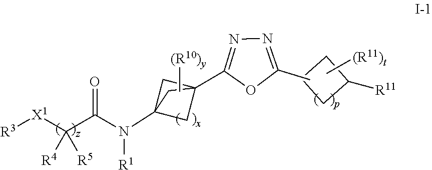

wherein p, t, x, y, z, X, R¹, R³, R⁴, R⁵, R¹⁰, and R¹¹ are as described herein.

In certain embodiments, the processes described herein are directed to making compounds of Formula I:

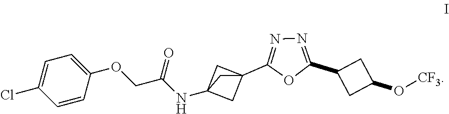

In some embodiments, the processes described herein avoid malodorous reactions or intermediates, improve purity, or provide salt or solid (e.g., crystalline) forms which allows the process to avoid oils or other liquid forms which may render the subsequent purification process or handling difficult.

### BRIEF DESCRIPTION OF THE DRAWINGS

FIG. 1 is the X-ray Powder Diffraction (XRPD) pattern of t-butylamine (TBA) salt of Compound of Formula VIa-1a, Form A (also referred to as Formula VII-1a, Form A).

FIG. 2 is the Thermogravimetry Analysis (TGA) and Differential Scanning Calorimetry (DSC) of TBA salt of Compound of Formula VIa-1a, Form A.

FIG. 3 is the XRPD pattern of dicyclohexylamine (DCHA) salt of Compound of Formula VIa-1a, Form A (also referred to as Formula VII-1b, Form A).

FIG. 4 is the TGA and DSC of DCHA salt of Compound of Formula VIa-1a, Form A.

FIG. 5 is the XRPD of L-Arginine salt of Compound of Formula VIa-1a, Form A (also referred to as Formula VII-1c, Form A).

FIG. 6 is the TGA and DSC of L-Arginine salt of Compound of Formula VIa-1a, Form A.

FIG. 7 is the XRPD of tromethamine (TMA) salt of Compound of Formula VIa-1a, Form A (also referred to as Formula VII-1d, Form A).

FIG. 8 is the TGA and DSC of TMA salt of Compound of Formula VIa-1a, Form A (also referred to as Formula VII-1d, Form A).

FIG. 9 is the DSC of 4-chlorophenoxyacetic acid salt of the Compound of Formula XI-1a Form A.

FIG. 10 is the XRPD of 4-chlorophenoxyacetic acid salt of the Compound of Formula XI-1a Form A.

FIG. 11 is the XRPD of Compound of Formula XI-1a, free base Form A.

FIG. 12 is the DSC of Compound of Formula XI-1a, free base Form A.

FIG. 13 is the XRPD of dioxalate salt of Compound of Formula XI-1a Form A.

FIG. 14 is the DSC of dioxalate salt of the Compound of Formula XI-1a Form A.

FIG. 15 is the crystal structure of 4-chlorophenoxyacetic acid salt of Compound of Formula XI-1a determined by microED.

FIG. 16 is the unit cell structure of 4-chlorophenoxyacetic acid salt of Compound of Formula XI-1a determined by microED.

FIG. 17 is a single crystal X-ray structure of Compound of Formula VII-1a.

FIG. 18 is the unit cell structure of Compound of Formula VII-1a.

### 1. DEFINITIONS

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl) or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl) or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkoxyalkyl” refers to the group “alkyl-O-alkyl”.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aminoalkyl” refers to the group “-alkyl-NR^{y}R^{z},” wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylalkyl” or “Aralkyl” refers to the group “aryl-alkyl-”.

“Carbamoyl” refers to both an “O-carbamoyl” group which refers to the group —OC(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Cyanoalkyl” refers to refers to an alkyl group as defined above, wherein one or more hydrogen atoms are replaced by a cyano (—CN) group.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkoxy” refers to “—O-cycloalkyl.”

“Cycloalkylalkyl” refers to the group “cycloalkyl-alkyl-”.

“Cycloalkylalkoxy” refers to “—O-alkyl-cycloalkyl.”

“Guanidino” refers to —NR^{y}C(═NR^{z})(NR^{y}R^{z}), wherein each R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Hydrazino” refers to —NHNH₂.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more hydrogen atoms are replaced by a halogen.

“Hydroxyalkyl” refers to an alkyl group as defined above, wherein one or more hydrogen atoms are replaced by a hydroxy group.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroalkylene” refers to a divalent alkyl group (i.e., alkylene) in which one or more of the carbon atoms (and any associated hydrogen atoms) are each independently replaced with the same or different heteroatomic group. The term “heteroalkylene” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkylene groups include, e.g., —CH₂OCH₂—, —CH(CH₃)OCH₂—, —CH₂CH₂OCH₂—, —CH₂CH₂OCH₂CH₂OCH₂—, —CH₂SCH₂—, —CH(CH₃)SCH₂—, —CH₂CH₂SCH₂—, —CH₂CH₂SCH₂CH₂SCH₂—, —CH₂S(O)₂CH₂—, —CH(CH₃)S(O)₂CH₂—, —CH₂CH₂S(O)₂CH₂—, —CH₂CH₂S(O)₂CH₂CH₂OCH₂—, —CH₂NR^{y}CH₂—, —CH(CH₃)NR^{y}CH₂—, —CH₂CH₂NR^{y}CH₂—, —CH₂CH₂NR^{y}CH₂CH₂NR^{y}CH₂—, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-”.

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Metal of closed shell” refers to a metal element where all valence electrons of the metal element are paired. Such a metal may exist as a cation (or ion) M^{W+}, wherein W denotes the charge and can be for example 1, 2, or 3. The metal cation may be, for example, a monovalent ion (e.g. having a +1 charge), a divalent ion (e.g. having a +2 charge), or a trivalent ion (e.g. having a +3 charge). For example, the ion M^{W+} may be, sodium ion (Na⁺), lithium ion (Li⁺), zinc ion (Zn²⁺), calcium ion (Ca²⁺), manganese ion (Mg²⁺), aluminum ion (Al³⁺), etc. In other words, the metal of closed shell may be Na, Li, Zn, Ca, Mn, Al, etc.

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more hydrogen atoms on the designated atom or group may or may not be replaced by a moiety other than hydrogen.

The term “substituted” refers to any of the above alkyl, alkenyl, alkynyl, cycloalkyl, heterocyclyl, aryl or heteroaryl groups in which one or more hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃, —OCF₃, N-oxide or —Si(R^{y})₃ wherein each R^{y} is independently hydrogen, alkyl, alkenyl, alkynyl, heteroalkyl, cycloalkyl, aryl, heteroaryl or heterocyclyl. In certain embodiments, “substituted” also means any of the above groups in which one or more hydrogen atoms are replaced with —C(═O)R^{g}, —C(═O)OR^{g}, —C(═O)NR^{g}R^{h}, —CH₂SO₂R^{g}, —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl) substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds described in this disclosure or prepared by processes of this disclosure include amino and/or carboxyl groups or groups similar thereto, and therefore may be prepared as acid and/or base salts.

The term “salt,” as used herein, denotes acidic salts formed with inorganic and/or organic acids, or basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. In certain embodiments, the salt comprises an amine, such as, but not limited to, t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, or dibenzylamine. In some embodiments, the salt comprises a cation, such as, but not limited to, magnesium, sodium, potassium, calcium, zinc, lithium, cesium, tetramethylammonium, or ammonium. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

The acid salt and base salt may be pharmaceutically acceptable salts or physiologically acceptable salts as defined below.

Provided are also a pharmaceutically acceptable salt, isotopically enriched analog, deuterated analog, stereoisomer, mixture of stereoisomers, and prodrugs of the compounds described herein, as well as processes for make the same. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases (e.g., an amine) include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂ (substituted alkyl)), di(substituted alkyl) amines (i.e., HN (substituted alkyl)₂), tri(substituted alkyl) amines (i.e., N (substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂ (substituted alkenyl)), di(substituted alkenyl) amines (i.e., HN (substituted alkenyl)₂), tri(substituted alkenyl) amines (i.e., N (substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. Therefore, the term “solvate” encompasses “hydrate.”

As used herein, the term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds or their pharmaceutically acceptable salts described here or provided by processes described here, may include an asymmetric center and may thus give rise to enantiomers, diastereomers, and other stereoisomeric forms that may be defined, in terms of absolute stereochemistry, as (R)- or (S)- or, as (D)- or (L)-for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The phrase “substantially as shown in FIG.” as applied to DSC thermograms is meant to include a variation of ±3° Celsius and as applied to TGA is meant to include a variation of ±2% in weight loss.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present disclosure and are disclosed herein just as if each and every such sub-combination was individually and explicitly disclosed herein.

### 2. PROCESSES

The processes described herein provide improved manufacturability for compounds useful in treating various diseases. The processes described herein are performed using suitable reactions conditions and optionally one or more protecting groups as needed.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, one or more protecting groups, or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Exemplary reaction conditions sufficient for performing the chemical transformations provided herein can be found throughout, and in particular, the examples below. It is also contemplated that the reaction conditions can include reagents in addition to those listed in the specific reaction.

The term “protecting group” refers to those groups intended to protect a given atom or functional group against undesirable reactions during synthetic procedures and includes, but is not limited to, silyl ethers, such as 2-(trimethylsilyl)ethoxymethyl (SEM) ether, or alkoxymethyl ethers, such as methoxymethyl (MOM) ether, tert-butoxymethyl (BUM) ether, benzyloxymethyl (BOM) ether or methoxyethoxymethyl (MEM) ether. Additional protecting groups include, tert-butyl, acetyl, benzyl, benzyloxycarbonyl (carbobenzyloxy, CBZ), p-methoxybenzyloxycarbonyl, p-nitrobenzyloxycarbonyl, tert-butoxycarbonyl (BOC), trifluoroacetyl, and the like. Certain protecting groups may be preferred over others due to their convenience or relative ease of removal, or due to their stereospecific effects in subsequent steps of the process. Additional suitable amino protecting groups are taught in T. W. Greene and P. G. M. Wuts, Protecting Groups in Organic Synthesis, Fifth Edition, Wiley, New York, 2014, and references cited therein which are all incorporated by reference in its entirety.

The term “contacting” or “contact” refers to the process of bringing into contact at least two distinct species such that they can interact with each other, such as in a non-covalent or covalent binding interaction or binding reaction. It should be appreciated, however, the resulting complex or reaction product can be produced directly from an interaction or a reaction between the added reagents or from an intermediate from one or more of the added reagents or moieties, which can be produced in the contacting mixture.

The term “leaving group” refers to an atom (or a group of atoms) with electron withdrawing ability that can be displaced as a stable species, taking with it the bonding electrons. Examples of suitable leaving groups include halides (e.g., Br, Cl, I), sulfonate esters (e.g., triflate, mesylate, tosylate, and brosylate), and nitrophenols.

### 3. ABBREVIATIONS

| Abbreviation | Meaning |
|---|---|
| ACN or MeCN | Acetonitrile |
| ° C. | Degree Celsius |
| DBDMH | 1,3-dibromo-5,5-dimethylhydantoin |
| DCHA | Dicyclohexylamine |
| DCM | Dichloromethane |
| DMF | Dimethylformamide |
| DIPEA | Diisopropylethylamine |
| DSC | Differential Scanning Calorimetry |
| aq. | Aqueous |
| Eq | Equivalent |
| g | Grams |
| h | Hour |
| M | Molar |
| Me | Methyl (CH₃) |
| MHz | Megahertz |
| MicroED | Microcrystal electron diffraction |
| NBS | N-bromosuccinimide |
| MTBE | Methyl tert butyl ether |
| nm | Nanometer |
| μM | Micromolar |
| RT | Room Temperature |
| TBA | t-butylamine |
| TGA | Thermogravimetry Analysis |
| THF | Tetrahydrofuran |
| TMA | tromethamine |
| XRPD | X-ray Powder Diffraction |

### 4. METHODS

Provided herein is a process for preparing a compound of Formula I-1:

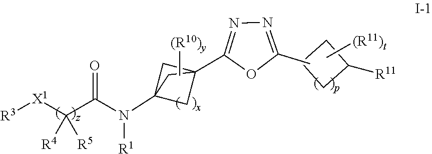

or a stereoisomer or mixture of stereoisomers thereof, or salt of each thereof, according to Scheme 1:
wherein
x is 1 or 2;
p is 0, 1, 2, 3, 4, 5, 6, or 7;
t is 0, 1, 2, 3, 4, or 5;
z is 0 or 1, provided that when z is 0 and X¹ is 0, then R³ is not alkyl;
X¹ is O, NR⁹, or a bond;
R¹ is hydrogen, C₁₋₁₂ alkyl, C₂₋₁₂ alkenyl, C₂₋₁₂ alkynyl, C₃₋₁₀ cycloalkyl, or heterocyclyl, each of which, other than hydrogen, is optionally substituted with one or more halo, oxo, acetyl, amino, hydroxyl, or C₁₋₁₂ alkyl, or R¹ and R⁵ together form a heterocyclyl ring;
R³ is hydrogen, C₁₋₁₂ alkyl, C₂₋₁₂ alkenyl, C₂₋₁₂ alkynyl, C₃₋₁₀ cycloalkyl, heterocyclyl, aryl, or heteroaryl, each of which, other than hydrogen, is optionally substituted with one or more R¹¹;
each of R⁴ and R⁵ is independently hydrogen, C₁₋₁₂ alkyl, C₂₋₁₂ alkenyl, or C₂₋₁₂ alkynyl, each of which, other than hydrogen, is independently optionally substituted with one or more halo, oxo, acetyl, amino, or hydroxyl;
or R³ and R⁴, together with the atoms to which they are attached, join to form a C₃₋₁₀ cycloalkyl, heterocyclyl, or heteroaryl, each of which is optionally substituted with one or more R¹¹;
or R⁴ and R⁵, together with the atoms to which they are attached, join to form a C₃₋₁₀ cycloalkyl or heterocyclyl, each of which is optionally substituted with one or more R¹¹;
each of R⁶, R⁷ and R¹ is independently hydrogen, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R²⁰, —C(O)OR²⁰, —C(O)NR²OR²¹, —S(O)₁₋₂R²⁰, or —S(O)₁₋₂NR²⁰, wherein each alkyl, alkenyl, alkynyl, cycloalkyl, heterocyclyl, aryl and heteroaryl of R⁶, R⁷, and R⁸ is independently optionally substituted with one or more R¹²; or
two of R⁶, R⁷ and R¹ are taken together with the atoms to which they are attached to form heterocyclyl independently optionally substituted by one or more halo, oxo, or C₁₋₁₂ alkyl independently optionally substituted by one or more oxo, halo, hydroxyl, or amino;
R⁹ is hydrogen, C₁₋₁₂ alkyl, C₂₋₁₂ alkenyl, C₂₋₁₂ alkynyl, C₃₋₁₀ cycloalkyl or heterocyclyl, each of which, other than hydrogen, is optionally substituted with one or more halo, oxo, acetyl, amino, hydroxyl, or C₁₋₁₂ alkyl;
each R¹⁰ is independently halo, C₁₋₁₂ alkyl, or C₁₋₁₂ haloalkyl;
y is 0, 1, 2, 3, 4, 5, 6, 7, or 8;
each R¹¹ is independently halo, cyano, nitro, oxo, —OR⁶, —SR⁶, —SF₅, —NR⁶R⁷, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R⁶, —C(O)OR⁶, —OC(O)OR⁶, —OC(O)R⁶, —C(O)NR⁶R⁷, —OC(O)NR⁶R⁷, —NR⁶C(O)NR⁷R⁸, —S(O)₁₋₂R⁶, —S(O)₁₋₂NR⁶, —NR⁶S(O)₁₋₂R⁷, —NR⁶S(O)₁₋₂NR⁷R⁸, —NR⁶C(O)R⁷, or —NR⁶C(O)OR⁷, wherein each alkyl, alkenyl, alkynyl, cycloalkyl, heterocyclyl, aryl and heteroaryl of R¹¹ is independently optionally substituted with one or more R¹²;
each R¹² is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³OR³¹, —OC(O)NR³OR³¹, —NR³OC(O)NR³OR³¹, —S(O)₁₋₂R³⁰, —S(O)₁₋₂NR³⁰, —NR³⁰S(O)₁₋₂R³¹, —NR³⁰S(O)₁₋₂NR³⁰R³¹, —NR³⁰C(O)R³¹, or —NR³⁰C(═O)OR³¹, wherein each alkyl, alkenyl, alkynyl, cycloalkyl, heterocyclyl, aryl and heteroaryl of R¹² is independently optionally substituted with one or more halo or C₁₋₁₂ alkyl independently optionally substituted by one or more oxo, halo, hydroxyl, or amino;
each R²⁰ and R²¹ is independently hydrogen or C₁₋₁₂ alkyl independently optionally substituted with one or more oxo, halo, hydroxyl, or amino; or
R²⁰ and R²¹ are taken together with the atoms to which they are attached to form heterocyclyl independently optionally substituted by one or more halo or C₁₋₁₂ alkyl independently optionally substituted by one or more oxo, halo, hydroxyl, or amino; and
each R³⁰ and R³¹ is independently hydrogen or C₁₋₁₂ alkyl independently optionally substituted with one or more oxo, halo, hydroxyl, or amino; or
R³⁰ and R³¹ are taken together with the atoms to which they are attached to form heterocyclyl independently optionally substituted by one or more halo or C₁₋₁₂ alkyl independently optionally substituted by one or more oxo, halo, hydroxyl, or amino.

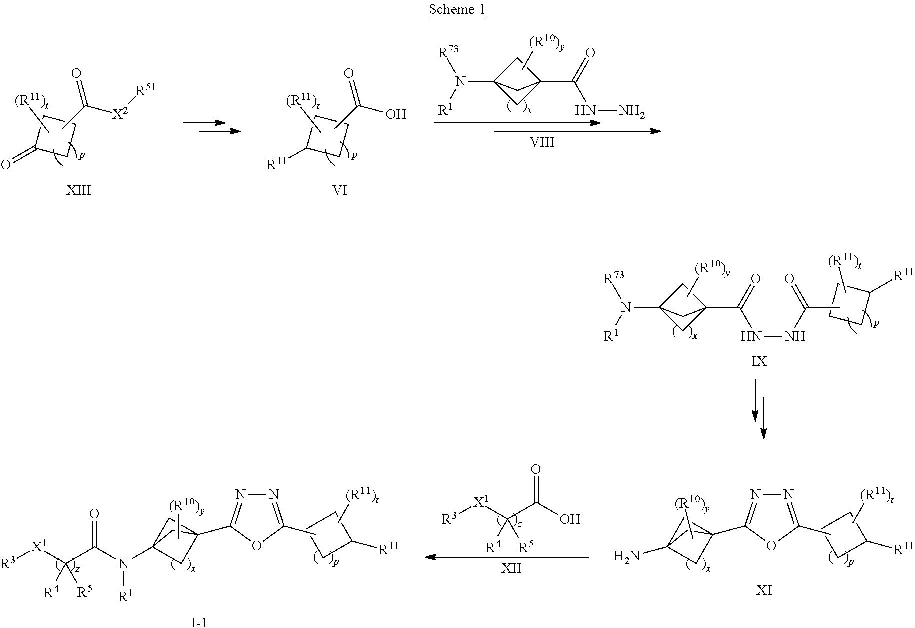

wherein:
x is 1 or 2;
p is 0, 1, 2, 3, 4, 5, 6, or 7;
t is 0, 1, 2, 3, 4, or 5;
z is 0 or 1, provided that when z is 0 and X¹ is 0, then R³ is not alkyl;
X¹ is O, NR⁹, or a bond;
X² is O or NR⁵³
R¹ is hydrogen, C₁₋₁₂ alkyl, C₂₋₁₂ alkenyl, C₂₋₁₂ alkynyl, C₃₋₁₀ cycloalkyl, or heterocyclyl, each of which, other than hydrogen, is optionally substituted with one or more halo, oxo, acetyl, amino, hydroxyl, or C₁₋₁₂ alkyl, or R¹ and R⁵ together form a heterocyclyl ring;
R³ is hydrogen, C₁₋₁₂ alkyl, C₂₋₁₂ alkenyl, C₂₋₁₂ alkynyl, C₃₋₁₀ cycloalkyl, heterocyclyl, aryl, or heteroaryl, each of which, other than hydrogen, is optionally substituted with one or more R¹¹;
each of R⁴ and R⁵ is independently hydrogen, C₁₋₁₂ alkyl, C₂₋₁₂ alkenyl, or C₂₋₁₂ alkynyl, each of which, other than hydrogen, is independently optionally substituted with one or more halo, oxo, acetyl, amino, or hydroxyl;
or R³ and R⁴, together with the atoms to which they are attached, join to form a C₃₋₁₀ cycloalkyl, heterocyclyl, or heteroaryl, each of which is optionally substituted with one or more R¹¹;
or R⁴ and R⁵, together with the atoms to which they are attached, join to form a C₃10 cycloalkyl or heterocyclyl, each of which is optionally substituted with one or more R¹¹;
each of R⁶, R⁷ and R¹ is independently hydrogen, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R²⁰, —C(O)OR²⁰, —C(O)NR²⁰R²¹, —S(O)₁₋₂R²⁰, or —S(O)₁₋₂NR²⁰, wherein each alkyl, alkenyl, alkynyl, cycloalkyl, heterocyclyl, aryl and heteroaryl of R⁶, R⁷, and R⁸ is independently optionally substituted with one or more R¹²; or
two of R⁶, R⁷ and R¹ are taken together with the atoms to which they are attached to form heterocyclyl independently optionally substituted by one or more halo, oxo, or C₁₋₁₂ alkyl independently optionally substituted by one or more oxo, halo, hydroxyl, or amino;
R⁹ is hydrogen, C₁₋₁₂ alkyl, C₂₋₁₂ alkenyl, C₂₋₁₂ alkynyl, C₃₋₁₀ cycloalkyl or heterocyclyl, each of which, other than hydrogen, is optionally substituted with one or more halo, oxo, acetyl, amino, hydroxyl, or C₁₋₁₂ alkyl;
each R¹⁰ is independently halo, C₁₋₁₂ alkyl, or C₁₋₁₂ haloalkyl;
y is 0, 1, 2, 3, 4, 5, 6, 7, or 8;
each R¹¹ is independently halo, cyano, nitro, oxo, —OR⁶, —SR⁶, —SF₅, —NR⁶R⁷, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R⁶, —C(O)OR⁶, —OC(O)OR⁶, —OC(O)R⁶, —C(O)NR⁶R⁷, —OC(O)NR⁶R⁷, —NR⁶C(O)NR⁷R⁸, —S(O)₁₋₂R⁶, —S(O)₁₋₂NR⁶, —NR⁶S(O)₁₋₂R⁷, —NR⁶S(O)₁₋₂NR⁷R⁸, —NR⁶C(O)R⁷ or —NR⁶C(O)OR⁷, wherein each alkyl, alkenyl, alkynyl, cycloalkyl, heterocyclyl, aryl, and heteroaryl of R¹¹ is independently optionally substituted with one or more R¹²;
each R¹² is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³OR³¹, —OC(O)NR³OR³¹, —NR³OC(O)NR³OR³¹, —S(O)₁₋₂R³⁰, —S(O)₁₋₂NR³⁰, —NR³⁰S(O)₁₋₂R³¹, —NR³⁰S(O)₁₋₂NR³⁰R³¹, —NR³⁰C(O)R³¹ or —NR³⁰C(═O)OR³¹, wherein each alkyl, alkenyl, alkynyl, cycloalkyl, heterocyclyl, aryl, and heteroaryl of R¹² is independently optionally substituted with one or more halo or C₁₋₁₂ alkyl independently optionally substituted by one or more oxo, halo, hydroxyl, or amino;
each R²⁰ and R²¹ is independently hydrogen or C₁₋₁₂ alkyl independently optionally substituted with one or more oxo, halo, hydroxyl, or amino; or
R²⁰ and R²¹ are taken together with the atoms to which they are attached to form heterocyclyl independently optionally substituted by one or more halo or C₁₋₁₂ alkyl independently optionally substituted by one or more oxo, halo, hydroxyl, or amino;
each R³⁰ and R³¹ is independently hydrogen or C₁₋₁₂ alkyl independently optionally substituted with one or more oxo, halo, hydroxyl, or amino; or
R³⁰ and R³¹ are taken together with the atoms to which they are attached to form heterocyclyl independently optionally substituted by one or more halo or C₁₋₁₂ alkyl independently optionally substituted by one or more oxo, halo, hydroxyl, or amino;
R⁵¹ is C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino; and
R⁷³ is hydrogen, a protecting group, or

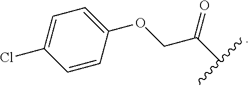 [SMILES: *C(=O)COc1ccc(Cl)cc1]

As illustrated in Scheme 1 above, in some embodiments, a compound of Formula XIII, or salt thereof, is converted into a carboxylic acid VI, or salt thereof. The carboxylic acid VI, or salt thereof, reacts with a hydrazide of Formula VIII, or salt thereof, under a condensation condition to provide a compound of Formula IX, or salt thereof. The compound of Formula IX, or salt thereof, then undergoes intramolecular ring closure reaction to provide the compound of Formula XI, or salt thereof. Finally, the compound of Formula XI, or salt thereof, further condenses with the compound of Formula XII, or salt thereof to form the product compound of Formula I-1. These processes are described in more detail below.

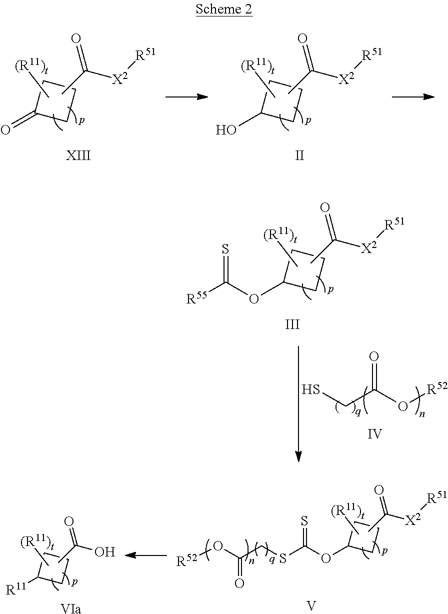

wherein p, t, and R¹¹ are as defined herein;
n is 0 or 1;
q is 1, 2, 3, 4, 5, 6, 7, 8, 9, 10, 11, or 12;
X² is O or NR⁵³;
R⁵¹ is C₁₋₁₂ alkyl or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is C₁₋₁₂ alkyl or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵³ is C₁₋₁₂ alkyl or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino; and
R⁵⁵ is selected from —Cl, —O-pyridyl, 1-imidazolyl, —N(alkyl)(alkyl), 1-pyridonyl, and 1-benzotriazolyl.

In some embodiments, at least one R¹¹ above is OR⁶. In some embodiments, at least one R¹¹ is —OCF₃.

In some embodiments, R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino. In some embodiments, R⁵¹ is C₁₋₁₂ alkyl or silyl, wherein the C₁₋₁₂ alkyl or silyl is optionally substituted with one or more halo or C₁₋₁₂ alkyl independently optionally substituted by one or more oxo, halo, hydroxyl, or amino. In some embodiments, R⁵¹ is hydrogen. In some embodiments, R⁵¹ is not hydrogen.

In some embodiments, X² is NR⁵³, and R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino. In some embodiments, X²—R⁵¹ is N-morpholino.

Referring to Scheme 2 above, in some embodiments, a compound of Formula VIa, or salt thereof, is prepared from the compound of Formula XIII, or salt thereof, following a series of steps. The compound of Formula VIa, or salt thereof, is an intermediate for the preparation of the compound of Formula I-1.

In some embodiments, provided here is a process for providing a compound of Formula II or a stereoisomer or mixture of stereoisomers thereof, or a salt thereof. The process comprises contacting a compound of Formula XIII, or a stereoisomer or mixture of stereoisomers thereof, or a salt thereof, with a reducing agent under conditions suitable to provide the compound of Formula II or a stereoisomer or mixture of stereoisomers thereof, or a salt thereof. In some embodiments, the conditions comprises a temperature below 0° C., such as about −25° C. to about −55° C., or about −35° C. to about −45° C. In some embodiments, the reducing agent is a chemical reductant. In some embodiments, the chemical reductant is selected from the group consisting of molecular hydrogen, elemental metal, metal hydride, transition metal complex with the metal in a reduced valence state, borane, borohydride, hydrazines, thiols, phosphites, and the like. In some embodiments, the chemical reductant is sodium borohydride or lithium aluminum hydride.

In some embodiments, the compound of Formula II is provided as a racemic mixture. In some embodiments, provided here is an enantiomer of the compound of Formula II. In some embodiments, the compound of Formula II is of a Formula IIa:

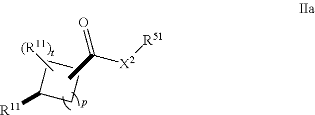

where the substituents are as defined above with respect to Scheme 2.

In some embodiments, the compound of Formula II is of a Formula IIa-1:

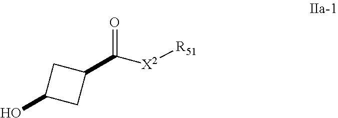

where the substituents are as defined above with respect to Scheme 2.

In some embodiments, the compound of Formula II or IIa-1 is of a Formula II-1:

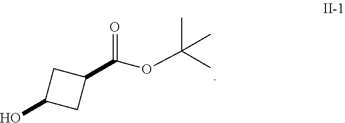

In some embodiments, the compound is not ethyl 3-[(methylthio)thioxomethoxy]cyclobutanecarboxylate. In some embodiments, the compound is not ethyl cis-3-[(methylthio)thioxomethoxy]cyclobutanecarboxylate.

In some embodiments, provided here is a process for providing a compound of Formula III or a stereoisomer or mixture of stereoisomers thereof, or a salt thereof. The process comprises contacting a compound of Formula II, IIa-1, II-1, or a stereoisomer or mixture of stereoisomers thereof, or a salt thereof, with an activating agent under reaction conditions sufficient to provide the compound of Formula III or a stereoisomer or mixture of stereoisomers thereof, or a salt thereof.

Any suitable activating reagent may be implemented. In some embodiments, the activating reagent is selected from the group consisting of thiophosgene, 1,1′-thiocarbonyldiimidazole, thiram, 1,1′-thiocarbonyldi-2(1H)-pyridone, 1,1′-(thiocarbonyl)bis-1H-benzotriazole, and di-2-pyridyl-thionocarbonate. In some embodiments, the activating reagent is 1,1′-thiocarbonyldiimidazole.

In some embodiments, the contacting of the compound of Formula II, IIa-1, II-1, or a stereoisomer or mixture of stereoisomers thereof, or the salt of each thereof, with the activating agent comprises a) optionally a reaction temperature of about 0° C. to about 60° C.; and/or b) optionally a reaction time duration of about 3 hours to about 48 hours. In some embodiments, the reaction temperature is about 15° C. to about 30° C. In some embodiments, the reaction time is about 3 hours to about 4 hours. In some embodiments, the reaction may proceed in any suitable solvent. In some embodiments, the solvent may be diethylether, methyl t-butylether, and the like.

In some embodiments, the activating reagent is carbon disulfide. In some embodiments, the contacting of the compound of Formula II, IIa-1, II-1, or a stereoisomer or mixture of stereoisomers thereof, or the salt of each thereof, with the activating agent comprises a) optionally a solvent of DMSO, b) optionally contacting the compound of Formula II, IIa-1, II-1, or a stereoisomer or mixture of stereoisomers thereof, or the salt of each thereof with 1,8-diazabicyclo[5.4.0]undec-7-ene (DBU) at a temperature of about 0° C. to about 50° C., such as about 10° C. to about 15° C., c) further contacting the mixture with carbon disulfide with the mixture at a temperature of about 0° C. to about 50° C., such as about 10° C. to about 15° C., d) optionally further contacting the mixture with methyl iodide at a temperature of about 0° C. to about 50° C., such as about 10° C. to about 30° C.

In some embodiments, provided here is a process for providing a compound of Formula V or a stereoisomer or mixture of stereoisomers thereof, or a salt thereof. The process comprises contacting a compound of Formula III or a stereoisomer or mixture of stereoisomers thereof, or a salt thereof, with a compound of Formula IV under the reaction conditions sufficient to provide a compound of Formula V or a stereoisomer or mixture of stereoisomers thereof, or a salt thereof.

In some embodiments, the compound of Formula IV is of a Formula IV-1:

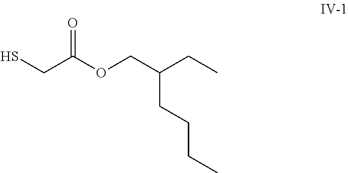

In some embodiments, provided here is a compound of Formula V, wherein the substituents are as defined above with respect to Scheme 2.

In some embodiments, provided here is the compound of Formula V as a racemic mixture. In some embodiments, provided here is an enantiomer of the compound of Formula V. In some embodiments, the compound of Formula V is of a Formula V-1:

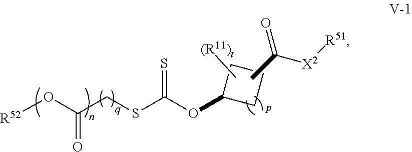

where the substituents are as defined above.

In some embodiments, the compound of Formula V is Formula V-2:

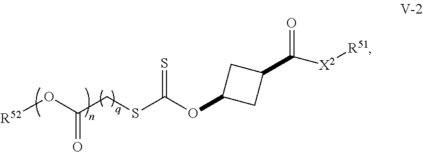

where the substituents are as defined above.

In some embodiments, provided is a method of preparing a compound of Formula V-2:

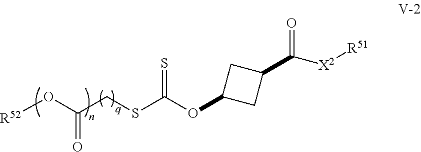

or a salt thereof, comprising contacting a compound of Formula IIa-1:

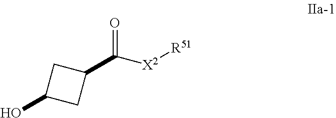

with an activating agent and then contacting with a compound of Formula IV:

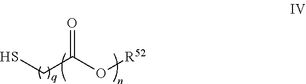

under reaction conditions sufficient to provide the compound of Formula V-2, wherein:
n is 0 or 1;
q is 1, 2, 3, 4, 5, 6, 7, 8, 9, 10, 11, or 12;
X² is O or NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided the compound is not ethyl cis-3-[(methylthio)thioxomethoxy]cyclobutanecarboxylate.

In some embodiments, X² is O.

In some embodiments, X² is NR⁵³.

In some embodiments, X² is NR⁵³, and R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino.

In some embodiments, R⁵¹ and R⁵³ together with the nitrogen atom join to form a -morpholino.

In some embodiments, R⁵¹ is C₁₋₁₂ alkyl.

In some embodiments, X² is O and R⁵¹ is C₁₋₁₂ alkyl.

In some embodiments, R⁵² is C₁₋₁₂ alkyl.

In some embodiments, n is 1. In some embodiments, n is 0.

In some embodiments, q is 1.

In some embodiments, R⁵² is substituted or unsubstituted C₄-C₁₂ alkyl.

In some embodiments, provided is a compound selected from the group consisting of:

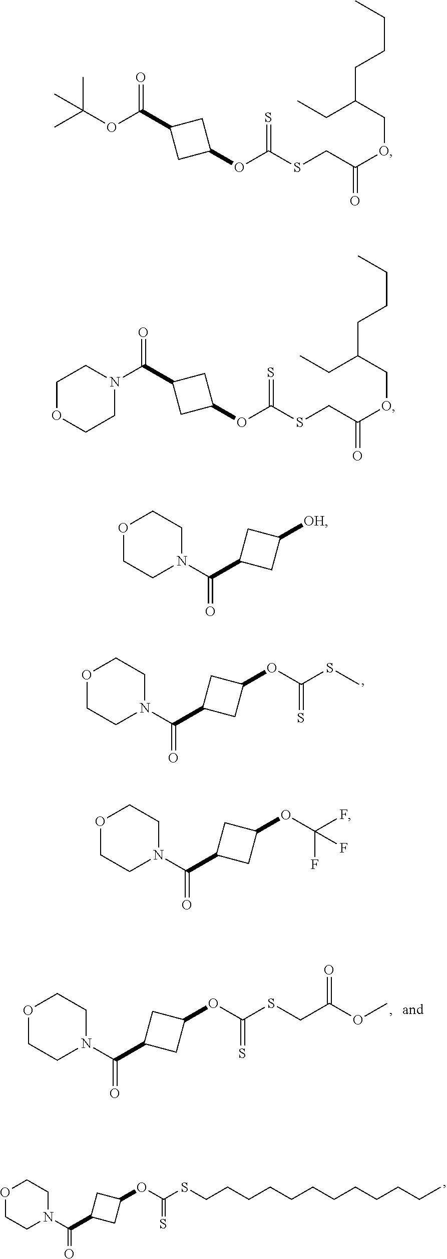

or a salt of each thereof.

In some embodiments, the compound of Formula V or the compound of Formula V-1 is Formula V-1a or a salt thereof:

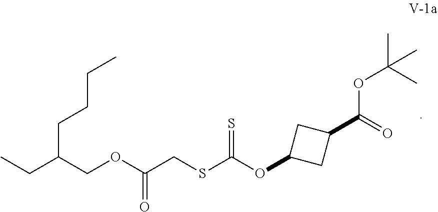

In some embodiments, the compound of Formula V or the compound of Formula V-1 is Formula V-1b or a salt thereof:

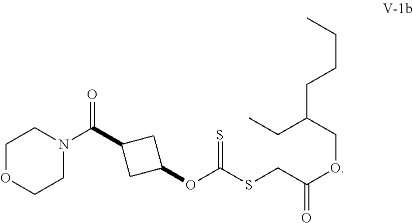

In some embodiments, the compound of Formula V or the compound of Formula V-1 is Formula V-1b or a salt thereof:

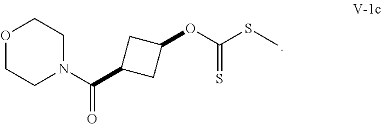 [SMILES: CSC(=S)O[C@H]1C[C@@H](C(=O)N2CCOCC2)C1]

In some embodiments, the compound of Formula V or the compound of Formula V-1 is Formula V-1b or a salt thereof:

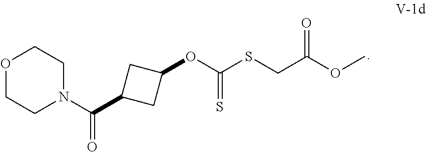 [SMILES: COC(=O)CSC(=S)O[C@H]1C[C@@H](C(=O)N2CCOCC2)C1]

In some embodiments, the contacting with the compound of Formula IV comprises contacting under reaction conditions that comprise a) optionally a reaction temperature of about 20° C. to about 80° C.; and/or b) optionally a reaction time duration of about 1 hour to about 48 hours. In some embodiments, the reaction temperature is about 40° C. to about 45° C. In some embodiments, the reaction time is about 1 hour to about 18 hours. In some embodiments, the reaction may proceed in any suitable solvent. In some embodiments, the solvent may be diethylether, methyl t-butylether, and the like.

Accordingly, as described above, provided here is a process of preparing a compound of Formula V, V-1, or V-2. The process comprises contacting a compound of Formula II, IIa-1, II-1, or a stereoisomer or mixture of stereoisomers thereof, or a salt thereof, with an activating agent, and then contacting with a compound of Formula IV or IV-1 under reaction conditions sufficient to provide the compound of Formula V, V-1, or V-2, wherein the substituents and parameters are according to those defined above with respect to Scheme 2.

In some embodiments, provided here is a process of preparing a compound of Formula V-1a. The process comprises contacting a compound of Formula II-1 with an activating agent, and then contacting with a compound of Formula IV-1 under reaction conditions sufficient to provide the compound of Formula V-1a, wherein the substituents and parameters are according to those defined above with respect to Scheme 2.

In some embodiments, for the method described here with respect to any of the above processes, R⁵¹ is a branched alkyl.

In some embodiments, for the method described here with respect to any of the above processes, t is 0, and X² is O.

In some embodiments, for the method described here with respect to any of the above processes, at least one R¹¹ is OR⁶. Accordingly, the compound of Formula VI can be described as Formula VIa-1:

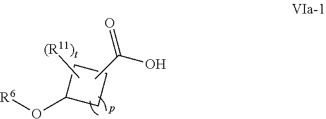

where the substituents are as defined above.

In some embodiments, provided here is a process for providing a compound of Formula VIa-1 or a stereoisomer or mixture of stereoisomers thereof, or a salt thereof. The process comprises contacting a compound of Formula V, V-1, V-2, or V1-a, or a stereoisomer or mixture of stereoisomers of each thereof, or a salt thereof, with a brominating reagent and a fluoride source under reaction conditions sufficient to provide the compound of Formula VIa or VIa-1.

In some embodiments, the compound of Formula V, V-1, V-2, or V1-a, or a stereoisomer or mixture of stereoisomers of each thereof, or a salt of each thereof, has been prepared using any of the methods described above.

In some embodiments, R⁶ is a substituted alkyl. In some embodiments, R⁶ is a haloalkyl. In some embodiments, R⁶ is trifluoromethyl (—CF₃). In some embodiments, provided here is a compound of (1s,3s)-3-(trifluoromethoxy)cyclobutane-1-carboxylic acid, or of Formula VIa-1a or a salt thereof:

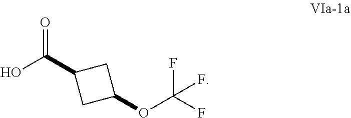

In some embodiments, provided here is a process for providing a compound of Formula VIa-1a. In some embodiments, the brominating reagent is 1,3-dibromo-5,5-dimethylhydantoin (DBDMH).

In some embodiments, the fluoride source is hydrogen fluoride (HF). In some embodiments, the fluoride source is HF-pyridine.

In some embodiments, the contacting with the brominating reagent and the fluoride source comprises introducing the compound of Formula V, V-1, V-2, or V1-a into a solution of the brominating reagent and the fluoride source at a temperature of less than about −25° C. In some embodiments, the contacting further comprises aging the resultant reaction mixture at room temperature. In some embodiments, the reaction may proceed in any suitable solvent. In some embodiments, the solvent may be DCM and the like.

In some embodiments, provided herein is a salt of the compound of Formula VIa, such as a salt of a compound of Formula VIa-1a, wherein the salt comprises an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a cation selected from magnesium, sodium, potassium, calcium, zinc, lithium, cesium, tetramethylammonium, and ammonium.

In some embodiments, provided herein is a method for preparing a salt of a compound of Formula VIa-1a:

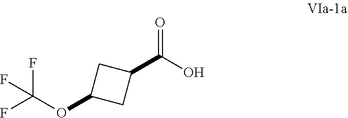

comprising contacting a compound of Formula V-2:

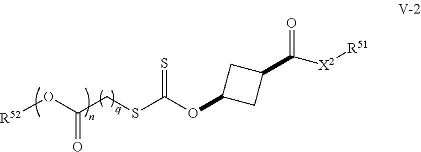

or a salt thereof, with an electrophilic bromine source and a fluorinating agent to provide the compound of Formula VIa-1a; and
contacting the compound of Formula VIa-1a with an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a base containing a cation selected from magnesium, sodium, potassium, calcium, zinc, lithium, cesium, tetramethylammonium, and ammonium; under reaction conditions sufficient to provide the compound of Formula V-2,
   wherein:
n is 0 or 1;
q is 1, 2, 3, 4, 5, 6, 7, 8, 9, 10, 11, or 12;
X² is O or NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided that when R⁵¹ is C₁₋₁₂ alkyl or silyl, or when X² is NR⁵³, the method comprises a hydrolysis step prior to forming the salt.

In some embodiments, the electrophilic bromine source comprises DBDMH (1,3-dibromo-5,5-dimethylhydantoin) or NBS (n-bromosuccinimide).

In some embodiments, the fluorinating agent is HF-pyridine.

In some embodiments, the hydrolysis step comprises a strong acid (i.e. HCl) at elevated temperature.

In some embodiments, X² is O.

In some embodiments, X² is NR⁵³.

In some embodiments, provided herein is a method for preparing a salt of a compound of Formula VIa-1a:

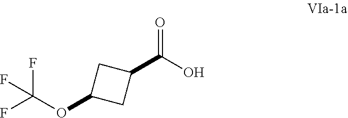

comprising contacting a compound of Formula V-2:

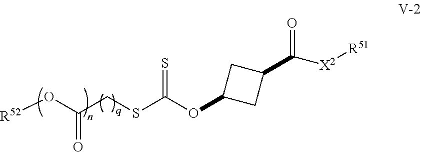

or a salt thereof, with an electrophilic bromine source and a fluorinating agent, followed by hydrolysis to form VIa-1a; and
contacting VIa-1a with an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a base containing a cation selected from magnesium, sodium, potassium, calcium, zinc, lithium, cesium, tetramethylammonium, and ammonium; under reaction conditions sufficient to provide the salt of VIa-1a:
n is 0 or 1;
q is 1, 2, 3, 4, 5, 6, 7, 8, 9, 10, 11, or 12;
X² is NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided the compound is not ethyl cis-3-[(methylthio)thioxomethoxy]cyclobutanecarboxylate.

In some embodiments, the base containing a cation is Mg(OH)₂ or NaOH.

In some embodiments, the salt of the compound of Formula VIa or VIa-1a is an amine salt (e.g., a salt comprising an amine (i.e., a nitrogen) as described herein). In some embodiments, provided here is a compound of a Formula VIIa:

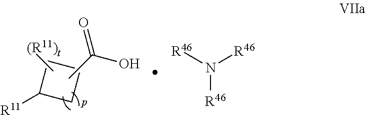

or a stereoisomer or mixture of stereoisomers thereof, wherein:
t is 0, 1, 2, 3, or 4;
each R⁴⁶ is independently hydrogen, C₁₋₆ alkyl, C₂₋₆ alkenyl, C₂₋₆ alkynyl, C₃₋₂₀ cycloalkyl, heterocyclyl, aryl, or heteroaryl, wherein each alkyl, alkenyl, alkynyl, cycloalkyl, heterocyclyl, aryl, and heteroaryl of R⁴⁶ is independently optionally substituted with one or more R¹²; and
other substituents are as defined above with respect to Scheme 2.

In some embodiments, p is 1, at least one R¹¹ is a halogen, and none of the R⁴⁶ are H.

In some embodiments, p is 1, at least one R¹¹ is a halogen, and one of the R⁴⁶ is H.

In some embodiments, p is 1, at least one R¹¹ is a halogen, and two of the R⁴⁶ are H.

In some embodiments, the N(R⁴⁶)₃ moiety in the Formula VIIa is selected from the group consisting of t-butylamine, L-lysine, piperazine, dicyclohexylamine, arginine, tromethamine, ethanolamine, dehydroabietylamine, and dibenzylamine.

In some embodiments, provided herein is a composition comprising the compound of Formula VIIa as a racemic mixture. In some embodiments, provided herein is a composition comprising the compound of Formula VIIa as a single diastereomer. In some embodiments, provided herein is a composition comprising predominantly the trans-isomer of the compound of Formula VIIa (e.g., >90% de, or >95% de, or >98% de, or greater than 99% de). In some embodiments, provided herein is a composition comprising predominantly the cis-isomer of the compound of Formula VIIa (e.g., >90% de, or >95% de, or >98% de, or greater than 99% de).

In some embodiments, the compound of Formula VIIa is Formula VII-1:

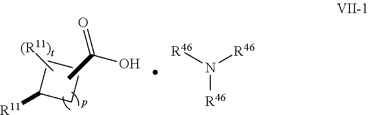

where the substituents are as defined above.

In some embodiments, the compound of Formula VII-1 is Formula VII-2a:

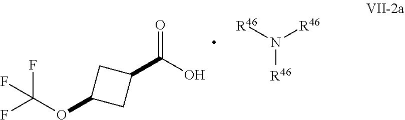

where the substituents are as defined above.

In some embodiments, provided herein is a method of preparing the compound of Formula VII-2a:

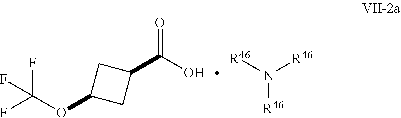

comprising contacting a compound of Formula VIa-1a:

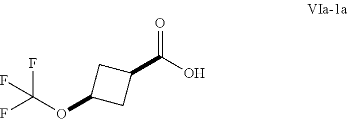

with an amine of Formula VIb:

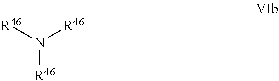

under reaction conditions sufficient to provide the compound of Formula VIIa,
wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

In some embodiments, provided here is a t-butylamine salt of (1s,3s)-3-(trifluoromethoxy)cyclobutane-1-carboxylic acid, or a compound having the structure of Formula VII-1a:

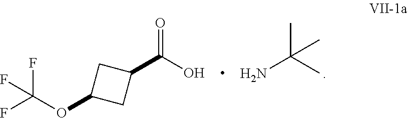

In some embodiments, provided here is a dicyclohexylamine salt of (1s,3s)-3-(trifluoromethoxy)cyclobutane-1-carboxylic acid, or a compound having the structure of Formula VII-1b:

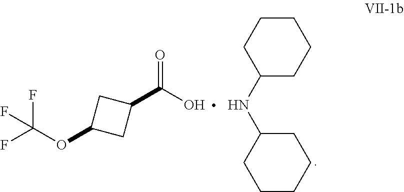 [SMILES: O=C(O)[C@H]1C[C@@H](OC(F)(F)F)C1 ; C1CCC(NC2CCCCC2)CC1]

In some embodiments, provided here is an L-arginine salt of (1s,3s)-3-(trifluoromethoxy)cyclobutane-1-carboxylic acid, or a compound having the structure of Formula VII-1c:

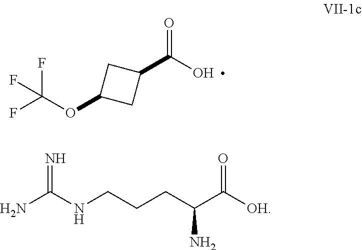 [SMILES: O=C(O)[C@H]1C[C@@H](OC(F)(F)F)C1 ; N=C(N)NCCC[C@H](N)C(=O)O]

In some embodiments, provided here is a tromethamine salt of (1s,3s)-3-(trifluoromethoxy)cyclobutane-1-carboxylic acid, or a compound having the structure of Formula VII-1d:

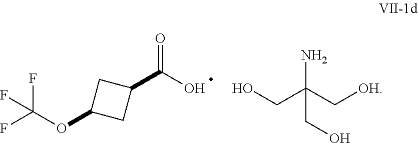 [SMILES: O=C(O)[C@H]1C[C@@H](OC(F)(F)F)C1 ; NC(CO)(CO)CO]

In some embodiments, the salt of the compound of Formula VIa is a metal salt. In some embodiments, provided here is a compound of a Formula VIIb:

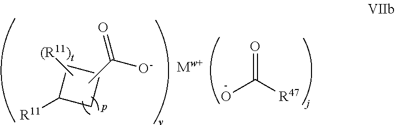

or a stereoisomer or mixture of stereoisomers thereof, wherein:
t is 0, 1, 2, 3, or 4;
w is 1, 2, or 3;
v is 1 or 2;
j is w-v, and is 0, 1, or 2;
M^{w+} is a cation;
each R⁴⁷ is C₁₋₆ alkyl; and
other substituents and parameters are as defined above with respect to Scheme 2.

Accordingly, the compound of a Formula VIIb is a metal salt of the compound of Formula VIa, and includes an anion form of the compound of Formula VIa, such as a monovalent anion of the compound of Formula VIa. In some embodiments, j is 0.

In some embodiments, the compound of a Formula VIIb may further include another anion. In other words, j may be non-zero. For example, the compound of a Formula VIIb may further include a carboxylate anion. In some embodiments, R⁴⁷ is a substituted or non-substituted alkyl. In some embodiments, R⁴⁷ is methyl. In some embodiments, j is 0 or 1.

In some embodiments, M is a metal of closed shell. In some embodiments, M is selected from the group consisting of Mg, Ca, Zn, Na, and Li. In some embodiments, the anion of the Formula VIa and the additional anion collectively balance the charge of the metal ion.

In some embodiments, provided here is the compound of Formula VIIb as a racemic mixture. In some embodiments, provided here is an enantiomer of the compound of Formula VIIb. In some embodiments, the compound of Formula VIIb is Formula VIIb-1:

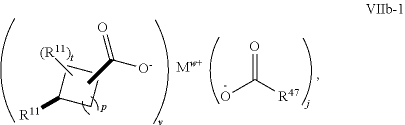

where the substituents are as defined above.

In some embodiments, the compound of Formula VIIb-1 is Formula VII-2b:

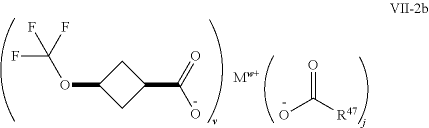

where the substituents are as defined above.

In some embodiments, with respect to the salt of the compound of Formula VIa, such as the compound of Formula VIIa or VIIb, at least one R¹¹ is OR⁶.

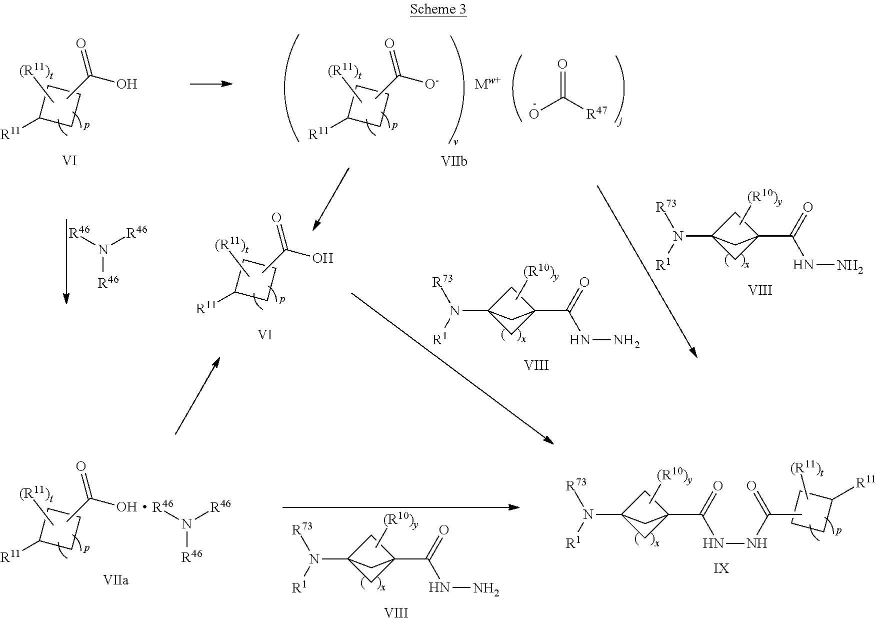

Referring to Scheme 3, in some embodiments, provided here is a method of preparing a compound of Formula IX or a stereoisomer or a mixture of stereoisomers thereof from the compound of Formula VI or a stereoisomer or a mixture of stereoisomers thereof, following a series of steps, wherein:
R¹ is hydrogen, C₁₋₁₂ alkyl, C₂₋₁₂ alkenyl, C₂₋₁₂ alkynyl, C₃₋₁₀ cycloalkyl, heterocyclyl, or trialkylsilyl, each of which, other than hydrogen, is optionally substituted with one or more halo, oxo, acetyl, amino, hydroxyl, or C₁₋₁₂ alkyl, or R¹ and R⁵ together form a heterocyclyl ring; and
R⁷³ is hydrogen, a protecting group, or a moiety of Formula

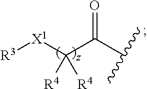

and
other substituents and parameters are as defined above with respect to Scheme 2.

In some embodiments, j is 0.

In some embodiments, the moiety of Formula

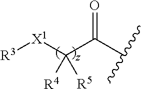

is

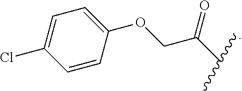 [SMILES: *C(=O)COc1ccc(Cl)cc1]

In some embodiments, provided here is a method of preparing the compound of Formula VIIa or a stereoisomer or a mixture of stereoisomers thereof, comprising contacting a compound of Formula VIa or a stereoisomer or a mixture of stereoisomers thereof with an amine of Formula VIb:

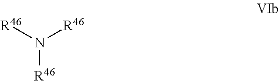

under reaction conditions sufficient to provide the compound of Formula VIIa.

In some embodiments, the contacting comprises contacting the compound of Formula VIa with the amine of Formula VIb at a stoichiometric ratio of about 1:0.8 to about 1:1.3. In some embodiments, the contacting comprises contacting the compound of Formula VIa with a slight excess of the amine of Formula VIb at a stoichiometric ratio of about 1:1.05 to about 1:1.3.

In some embodiments, provided here is a method of preparing a compound of Formula IX or a stereoisomer or mixture of stereoisomers thereof, or a solvate of each thereof, from the compound of Formula VIIa or a stereoisomer or mixture of stereoisomers thereof. In some embodiments, the process comprises purifying the compound of Formula VIIa or a stereoisomer or mixture of stereoisomers thereof to a sufficient purity (e.g., >95%, or >96%, or >97%, or >98%, or >99%, or between 95-100%). In some embodiments, the process comprises contacting the purified compound of Formula VIIa or a stereoisomer or mixture of stereoisomers thereof, with an acid under reaction conditions sufficient to provide the compound of Formula VI, or a stereoisomer or mixture of stereoisomers thereof; and contacting the compound of Formula VI, or a stereoisomer or mixture of stereoisomers thereof, with a compound of VIII under conditions sufficient to provide the compound of Formula IX or a stereoisomer or mixture of stereoisomers thereof.

Accordingly, the compound of Formula VI is generated from the compound of Formula VIIa prior to the reaction with the compound of Formula VIII. In some embodiments, the generation of the compound of Formula VI is conducted in situ prior to the introduction of the compound of Formula VIII. In some embodiments, this generation step ensures that the compound of Formula VI is provided in a sufficient purity prior to subsequent reactions. As discussed above, the compound of Formula VI generally exists in an oil form which may render its purification difficult thereby translating into low yields or purity in subsequent conversions. Here, using the compound of Formula VIIa as the starting material and later regenerate the compound of Formula VI when needed alleviates the purity challenge described above. In some embodiments, the purity of the compound of Formula VIIa is about 95%. In some embodiments, the purity of the compound of Formula VIIa is about 98%. In some embodiments, the purity of the compound of Formula VIIa is about 99%. In some embodiments, the purity of the compound of Formula VIIa is about 99.5%. In some embodiments, the purity of the compound of Formula VIIa is about 99.9%.

In some embodiments, the acid is a mineral acid. In some embodiments, the acid is sulfuric acid. In some embodiments, the reaction conditions under which the purified compound of Formula VIIa or a stereoisomer or mixture of stereoisomers thereof contacts with the acid comprises contacting at a temperature of about 20˜25° C.

In some embodiments, the reaction conditions providing the compound of Formula IX or a stereoisomer or mixture of stereoisomers thereof comprises introducing the compound of Formula VIII into a mixture of carbonyl diimidazole and the regenerated compound of Formula VI. In some embodiments, the reaction conditions include reacting at a temperature of about −10° C. to about 60° C. In some embodiments, the reaction conditions include reacting at a temperature of about 0° C. to about 30° C. The reaction may proceed in any suitable solvents. In some embodiments, the reaction conditions include reacting in DMF. In some embodiments, the reaction conditions include a reaction time of about 10 minutes to about 18 hours. In some embodiments, the reaction time is about 0.5 hour to about 4 hours.

In some embodiments, the step of generating the compound of Formula VI, or a stereoisomer or mixture of stereoisomers thereof is omitted, such that the process instead contacts the purified compound of Formula VIIa or a stereoisomer or mixture of stereoisomers thereof with the compound of VIII under conditions sufficient to provide the compound of Formula IX or a stereoisomer or mixture of stereoisomers thereof.

In some embodiments, provided here is a method of preparing a compound of Formula IX or a stereoisomer or mixture of stereoisomers thereof, or a solvate of each thereof, from the compound of Formula VIIb or a stereoisomer or mixture of stereoisomers thereof. In some embodiments, the method comprises contacting the compound of Formula VI with a suitable metal salt, such as magnesium acetate tetrahydrate, sodium hydroxide, calcium acetate, zinc acetate, lithium hydroxide, or similar metal salts in a suitable organic solvent, such as ethylacetate or toluene at a stoichiometric ratio of about 1:1. In some embodiments, the process comprises purifying the compound of Formula VIIb or a stereoisomer or mixture of stereoisomers thereof to a sufficient purity. In some embodiments, the purity of the compound of Formula VIIb is about 95%. In some embodiments, the purity of the compound of Formula VIIb is about 98%. In some embodiments, the purity of the compound of Formula VIIb is about 99%. In some embodiments, the purity of the compound of Formula VIIb is about 99.5%. In some embodiments, the purity of the compound of Formula VIIb is about 99.9%. Any suitable purification method is contemplated, including but not limited to recrystallization, and chromatography.

In some embodiments, the process comprises contacting the purified compound of Formula VIIb or a stereoisomer or mixture of stereoisomers thereof, with an acid under reaction conditions sufficient to provide the compound of Formula VI, or a stereoisomer or mixture of stereoisomers thereof; and contacting the compound of Formula VI, or a stereoisomer or mixture of stereoisomers thereof, with a compound of Formula VIII under conditions sufficient to provide the compound of Formula IX or a stereoisomer or mixture of stereoisomers thereof. Similar to the alternative route described above, the purification of the compound of Formula VIIb alleviates challenge associated with purifying the compound of Formula VI.

In some embodiments, the step of generating the compound of Formula VI, or a stereoisomer or mixture of stereoisomers thereof is omitted, such that the process instead contacts the purified compound of Formula VIIb or a stereoisomer or mixture of stereoisomers thereof with the compound of VIII under conditions sufficient to provide the compound of Formula IX or a stereoisomer or mixture of stereoisomers thereof.

In some embodiments, the acid is a mineral acid. In some embodiments, the acid is sulfuric acid. In some embodiments, the reaction conditions under which the purified compound of Formula VIIb or a stereoisomer or mixture of stereoisomers thereof contacts with the acid comprises contacting at a temperature of about 20 to 25° C.

In some embodiments, the reaction conditions providing the compound of Formula IX or a stereoisomer or mixture of stereoisomers thereof comprises introducing the compound of Formula VIII into a mixture of carbonyl diimidazole and the regenerated compound of Formula VI. In some embodiments, the reaction conditions include reacting at a temperature of about −10° C. to about 60° C. In some embodiments, the reaction conditions include reacting at a temperature of about 0° C. to about 30° C. The reaction may proceed in any suitable solvents. In some embodiments, the reaction conditions include reacting in DMF. In some embodiments, the reaction conditions include a reaction time of about 10 minutes to about 18 hours. In some embodiments, the reaction time is about 0.5 hour to about 4 hours.

In some embodiments, the compound of Formula VIII is Formula VIIIa:

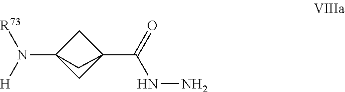

where the substituent R⁷³ are as defined above.

In some embodiments, provided here is the compound of Formula IX as a racemic mixture. In some embodiments, provided here is an enantiomer of the compound of Formula IX. In some embodiments, the compound of Formula IX is Formula IX-1:

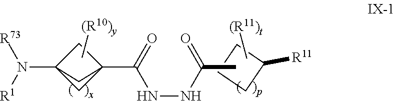

where the substituents are as defined above.

In some embodiments, the compound of Formula IX or IX-1 is Formula IX-2:

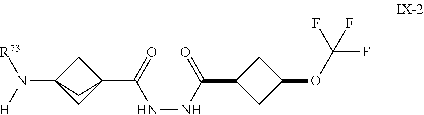

where the substituents are as defined above.

In some embodiments, provided here is a method of preparing a compound of Formula IX-2, or a solvate (for example, a hydrate) thereof, from the compound of Formula VII-2a. In some embodiments, the method comprises contacting a salt of a compound of Formula VIa-1a the compound of Formula VII-2a with an acid under reaction conditions sufficient to provide a compound of Formula VIa-1a, and contacting the compound of Formula VIa-1a with a compound of Formula VIIIa or a salt thereof under conditions sufficient to provide the compound of Formula IX-2. In some embodiments, the process comprises purifying the compound of Formula VII-2a to a sufficient purity. In some embodiments, the purity of the compound of Formula VII-2a is about 95%. In some embodiments, the purity of the compound of Formula VII-2a is about 98%. In some embodiments, the purity of the compound of Formula VII-2a is about 99%. In some embodiments, the purity of the compound of Formula VII-2a is about 99.5%. In some embodiments, the purity of the compound of Formula VII-2a is about 99.9%. Any suitable purification method is contemplated, including but not limited to recrystallization, and chromatography.

In some embodiments, the compound of Formula IX or IX-1 is Formula IX-1a:

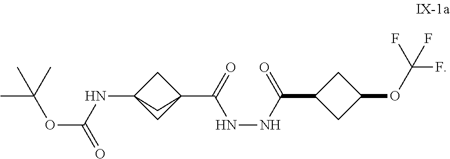

In some embodiments, provided here a solvate of the compound of Formula IX-1a. In some embodiments, provided here is a solvate, for example, a hydrate, of the compound of Formula IX-1a. In some embodiments, provided here is a hydrate, such as a monohydrate, of the compound of Formula IX-1a. In other words, the hydrate of the compound of Formula IX-1a includes the compound of Formula IX-1a with water at a stoichiometric ratio of about 1:1. In some embodiments, the monohydrate of the compound of Formula IX-1a provides superior stability in manufacturing process, as compared to, for example, an anhydrous form of the compound of Formula IX-1a.

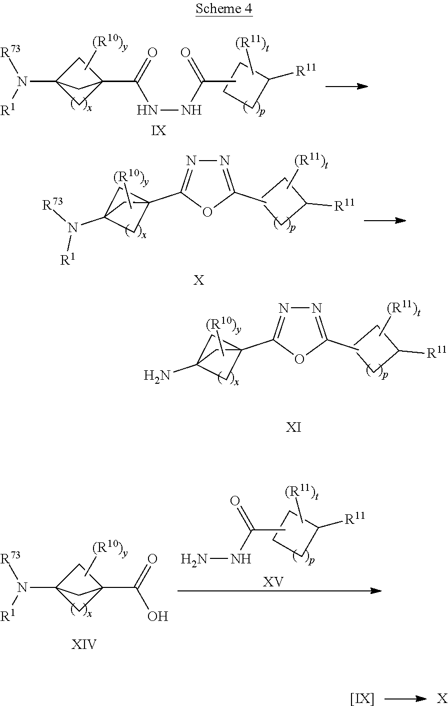

Referring to Scheme 4, where the substituents are as defined above, in some embodiments, provided here is a method of preparing a compound of Formula XI or a stereoisomer or mixture of stereoisomers thereof, or a salt of each thereof, from the compound of Formula IX or a stereoisomer or mixture of stereoisomers thereof, or a solvate of each thereof, through a two-step reaction. In some embodiments, the reaction starts from a solvate of the compound of Formula IX or a stereoisomer or mixture of stereoisomers thereof. In some embodiments, the solvate is a hydrate. In some embodiments, the solvate is a polar aprotic solvent, optionally N,N-dimethylformamide, N-methylpyrrolidone, or dimethylacetamide.

In some embodiments, provided here is a method of preparing a compound of Formula X or a stereoisomer or mixture of stereoisomers thereof, or a salt of each thereof, from the compound of Formula IX or a stereoisomer or mixture of stereoisomers thereof, or a solvate of each thereof. In some embodiments, the method comprises contacting the compound of Formula IX, or a stereoisomer or mixture of stereoisomers thereof, or a solvate of each thereof, with a dehydrating agent and with a base under reaction conditions sufficient to provide the compound of Formula X or a stereoisomer or mixture of stereoisomers thereof, or a salt of each thereof.

Alternatively, in some embodiments, provided here is a method of preparing a compound of Formula X or a stereoisomer or mixture of stereoisomers thereof, or a salt of each thereof, by contacting a compound of Formula XIV or a stereoisomer or mixture of stereoisomers thereof, or a solvate thereof, with a compound of Formula XV, under reaction conditions sufficient to provide the compound of Formula X or a stereoisomer or mixture of stereoisomers thereof, or a salt of each thereof. In some embodiments, the method comprises a dehydrating agent and a base.

In some embodiments, the dehydrating agent described above is selected from the group consisting of sulfonyl chloride, sulfonic anhydride, phosphonic anhydride, carbonic acid anhydride bearing electron withdrawing groups, and a mixture of triphenylphosphine with carbon tetrachloride.

In some embodiments, the dehydrating agent described above is selected from the group consisting of 4-toluenesulfonyl chloride, methanesulfonyl chloride, toluenesulfonic anhydride, methanesulfonyl anhydride, 2,4,6-tripropyl-1,3,5,2,4,6-trioxatriphosphinane 2,4,6-trioxide (or referred to here as tri-n-propylphosphonic anhydride), trifluoroacetic anhydride, and the mixture of triphenylphosphine with carbon tetrachloride.

In some embodiments, the acid is 4-toluenesulfonyl chloride.

In some embodiments, the base is potassium carbonate. In some embodiments, the base is an amine. In some embodiments, the base is N,N-diisopropylethylamine (DIPEA) or triethylamine (TEA). In some embodiments, the base is DIPEA. In some embodiments, the reaction conditions providing the compound of Formula X or a stereoisomer or mixture of stereoisomers thereof, or a salt of each thereof comprises a reacting in a solvent of ACN. In some embodiments, the reaction condition comprises a reaction temperature of about 0° C. to about 40° C. In some embodiments, the reaction condition comprises a reaction temperature of about 20° C. to about 25° C. In some embodiments, the ACN is anhydrous. For example, the ACN may have a water content of less than about 2%, or about 1%, or about 0.5%, or about 0.1%, or about 0.01%, or about 10 ppm, or about 1 ppm. In some embodiments, other solvents, such as other organic solvents including but not limited to DMF, N-Methylpyrrolidone (NMP), THF, methyl tetrahydrafuran (MeTHF), MTBE, isopropyl acetate (IPAc), do not produce satisfactory yield for this reaction.

In some embodiments, the compound of Formula IX or a stereoisomer or mixture of stereoisomers thereof, or a solvate of each thereof, used for the synthesis of the compound of Formula X or a stereoisomer or mixture of stereoisomers thereof, or a solvate of each thereof, is formed via the compound of Formula VIIa. In some embodiments, the compound of Formula IX or a stereoisomer or mixture of stereoisomers thereof, or a solvate of each thereof, used for the synthesis of the compound of Formula X or a stereoisomer or mixture of stereoisomers thereof, or a solvate of each thereof, is formed via the compound of Formula VIIb.

Accordingly, in some embodiments, provided here is a method for preparing the compound of Formula X, or a stereoisomer or mixture of stereoisomers thereof, or a salt of each thereof, comprising:
a) contacting a compound of Formula VIIa or a stereoisomer or mixture of stereoisomers thereof, with a first acid under reaction conditions sufficient to provide a compound of Formula VI, or a stereoisomer or mixture of stereoisomers thereof; and contacting the compound of Formula VI, or a stereoisomer or mixture of stereoisomers thereof, with a compound of VIII under conditions sufficient to provide a compound of Formula IX or a stereoisomer or mixture of stereoisomers thereof, or a solvate of each thereof;
b) contacting a compound of Formula VIIa or a stereoisomer or mixture of stereoisomers thereof, with the compound of VIII under conditions sufficient to provide a compound of Formula IX or a stereoisomer or mixture of stereoisomers thereof, or a solvate of each thereof;
c) contacting a compound of Formula VIIb or a stereoisomer or mixture of stereoisomers thereof, with the first acid under reaction conditions sufficient to provide a compound of Formula VI, or a stereoisomer or mixture of stereoisomers thereof; and contacting the compound of Formula VI, or a stereoisomer or mixture of stereoisomers thereof, with the compound of VIII under conditions sufficient to provide a compound of Formula IX or a stereoisomer or mixture of stereoisomers thereof, or a solvate of each thereof; or
d) contacting a compound of Formula VIIb or a stereoisomer or mixture of stereoisomers thereof with the compound of VIII under conditions sufficient to provide the compound of Formula IX or a stereoisomer or mixture of stereoisomers thereof, or a solvate of each thereof; and
contacting the compound of Formula IX or a stereoisomer or mixture of stereoisomers thereof, or a solvate of each thereof with a dehydrating agent and with a base under reaction conditions sufficient, to provide the compound of Formula X or a stereoisomer or mixture of stereoisomers thereof, or a salt of each thereof.

In some embodiments, the R⁷³ is a protecting group of the amine functional group. In some embodiments, the R⁷³ is tert-butoxycarbonyl (BOC). In some embodiments, R¹ is hydrogen. In some embodiments, R⁷³ is

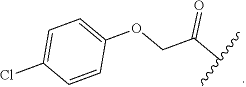 [SMILES: *C(=O)COc1ccc(Cl)cc1]

In some embodiments, the processes described with respect to Schemes 3 and 4 are conducted within a same reaction vessel without removing the reaction mixture. In some embodiments, the preparing of the compound of Formula IX, IX-1, or IX-1a and the preparing of the compound of Formula X are conducted within the same reaction vessel without removing the reaction mixture. In some embodiments, the processes described in Scheme 4 is conducted within a same reaction vessel without removing the reaction mixture.

In some embodiments, the preparing of the compound of Formula XIV, XV, IX, X, or IX-1a are conducted within the same reaction vessel without removing the reaction mixture or isolating the compound of Formula IX, or any intermediate provided by the methods described in Scheme 3 or 4.

In some embodiments, provided here is the compound of Formula X as a racemic mixture. In some embodiments, provided here is an enantiomer of the compound of Formula X. In some embodiments, the compound of Formula X is Formula X-1:

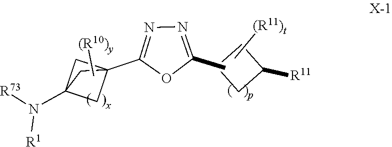

where the substituents are as defined above.

In some embodiments, the compound of Formula X-1 is Formula X-2:

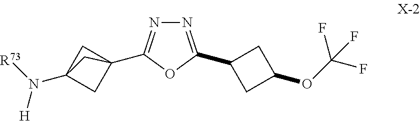

where the substituents are as defined above.

In some embodiments, provided here is a method of preparing a compound of Formula X-2 from the compound of Formula VII-2a. In some embodiments, the method comprises contacting the compound of Formula VII-2a with an acid under reaction conditions sufficient to provide a compound of Formula VIa-1a, contacting the compound of Formula VIa-1a with a compound of Formula VIIIa or a salt thereof under conditions sufficient to provide the compound of Formula IX-2, or a solvate (for example, a hydrate) of each thereof, and contacting the compound of Formula IX-2 with a dehydrating agent and with a base under reaction conditions sufficient, to provide the compound of Formula X-2 or a salt thereof. In some embodiments, the process comprises purifying the compound of Formula VII-2a to a sufficient purity. In some embodiments, the purity of the compound of Formula VII-2a is about 95%. In some embodiments, the purity of the compound of Formula VII-2a is about 98%. In some embodiments, the purity of the compound of Formula VII-2a is about 99%. In some embodiments, the purity of the compound of Formula VII-2a is about 99.5%. In some embodiments, the purity of the compound of Formula VII-2a is about 99.9%. Any suitable purification method is contemplated, including but not limited to recrystallization, and chromatography.

In some embodiments, provided here a compound of Formula X-1a:

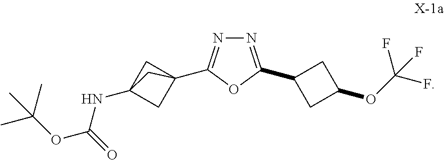

In some embodiments, provided here is a method of preparing a compound of Formula XI or a stereoisomer or mixture of stereoisomers thereof, or a salt of each thereof, from the compound of Formula X or a stereoisomer or mixture of stereoisomers thereof, or a salt of each thereof. In some embodiments, the method comprises contacting the compound of Formula X, or a stereoisomer or mixture of stereoisomers thereof, or a salt of each thereof, under deprotection conditions sufficient to provide the compound of Formula XI or a stereoisomer or mixture of stereoisomers thereof, or a salt of each thereof.

In some embodiments, the deprotection condition described with respect to the preparation of the compound of Formula IX comprises contacting the compound of Formula X with an acid. In some embodiments, the acid is generated in situ from an acid precursor in the presence of a protic compound. In some embodiments the acid precursor is acetyl chloride. In some embodiments, the protic compound is an alcohol. In some embodiments, the generation of the acid comprises generating at a temperature of about −30° C. to about 100° C. In some embodiments, the temperature is about −30° C. to about 60° C. In some embodiments, the generation of the acid comprises lowering the temperature to about 0° C. to about 5° C. and warming the reaction to a temperature of about 15° C. to about 25° C. In some embodiments, the temperature of the reaction mixture is increased to about 30° C. to about 60° C., such as about 40° C. to about 45° C., and stirred for a duration of at least about 2 hours. In some embodiments, the latter step of reaction at the higher temperature, such as at about 40° C. to about 45° C. provides significantly improved yield and/or reduced impurity content, as compared to other approaches, for example, maintaining the temperature at below about 30° C.

In some embodiments, the deprotection conditions comprise generating hydrogen chloride in situ. In some embodiments, the deprotection conditions further comprise, upon reaction completion, immediately neutralizing the generated acid that remains and isolating the compound of Formula X or the salt thereof. Other deprotection conditions, such as by contacting the intermediate X-1a with aqueous hydrochloric acid to remove the BOC group, often results in decomposition of the oxadiazole ring, leading to a low yield for the intermediate XI. By contrast, by implementing the deprotection and neutralization conditions described here, the ring decomposition is avoided, and the yield and/or purity of the compound of Formula XI may be substantially improved. As such, the deprotection conditions described here enables large scale industrial productions not previously conceivable.

In some embodiments, the contacting of the compound of Formula X, or a stereoisomer or mixture of stereoisomers thereof, or a salt of each thereof, under the deprotection conditions provides a free base form of the compound of Formula XI or a stereoisomer or mixture of stereoisomers thereof. In some embodiments, the contacting of the compound of Formula X, or a stereoisomer or mixture of stereoisomers thereof, or a salt of each thereof, under the deprotection conditions provides a salt of the compound of Formula XI or a stereoisomer or mixture of stereoisomers thereof. In some embodiments, the salt is a hydrochloride salt, oxalic acid salt, and 4-chlorophenoxyacetic acid salt. In some embodiments, every one equivalent of the hydrochloride salt includes two equivalents of chloride anion.

In some embodiments, every one equivalent of the oxalic acid salt includes two equivalents of oxalate anion. In some embodiments, every one equivalent of the 4-chlorophenoxyacetic acid salt includes one equivalent of 4-chlorophenoxyacetate anion.

Accordingly, in some embodiments, provided here is a method for preparing the compound of Formula XI, or a stereoisomer or mixture of stereoisomers thereof, or a salt of each thereof, comprising:
a) contacting a compound of Formula VIIa or a stereoisomer or mixture of stereoisomers thereof, with a first acid under reaction conditions sufficient to provide a compound of Formula VI, or a stereoisomer or mixture of stereoisomers thereof; and contacting the compound of Formula VI, or a stereoisomer or mixture of stereoisomers thereof, with a compound of VIII under conditions sufficient to provide a compound of Formula IX or a stereoisomer or mixture of stereoisomers thereof, or a solvate of each thereof;
b) contacting a compound of Formula VIIa or a stereoisomer or mixture of stereoisomers thereof, with the compound of VIII under conditions sufficient to provide a compound of Formula IX or a stereoisomer or mixture of stereoisomers thereof, or a solvate of each thereof;
c) contacting a compound of Formula VIIb or a stereoisomer or mixture of stereoisomers thereof, with the first acid under reaction conditions sufficient to provide a compound of Formula VI, or a stereoisomer or mixture of stereoisomers thereof; and contacting the compound of Formula VI, or a stereoisomer or mixture of stereoisomers thereof, with the compound of VIII under conditions sufficient to provide a compound of Formula IX or a stereoisomer or mixture of stereoisomers thereof, or a solvate of each thereof; or
d) contacting a compound of Formula VIIb or a stereoisomer or mixture of stereoisomers thereof with the compound of VIII under conditions sufficient to provide the compound of Formula IX or a stereoisomer or mixture of stereoisomers thereof, or a solvate of each thereof;
contacting the compound of Formula IX or a stereoisomer or mixture of stereoisomers thereof, or a solvate of each thereof, with a dehydrating agent and with a base under reaction conditions sufficient, to provide the compound of Formula X or a stereoisomer or mixture of stereoisomers thereof, or a salt of each thereof; and
contacting the compound of Formula X, or a stereoisomer or mixture of stereoisomers thereof, or a salt of each thereof, under deprotection conditions sufficient to provide the compound of Formula XI or a stereoisomer or mixture of stereoisomers thereof, or a salt of each thereof.

In some embodiments, provided here is the compound of Formula XI as a racemic mixture. In some embodiments, provided here is an enantiomer of the compound of Formula XI. In some embodiments, the compound of Formula XI is Formula XI-1:

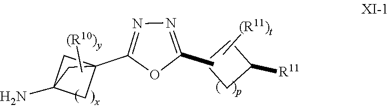

where the substituents are as defined above.

In some embodiments, provided here a compound of Formula XI-1a:

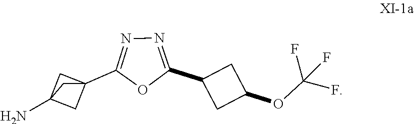

In some embodiments, with respect to any methods described above preparing a salt of the compound of Formula XI, Formula XI-1, or Formula XI-1a, the salt is a hydrochloride salt, oxalic acid salt, and 4-chlorophenoxyacetic acid salt. In some embodiments, the salt is 4-chlorophenoxyacetic acid salt. In some embodiments, the compound of Formula XI, Formula XI-1, or Formula XI-1a exists in the salt with respect to its counter ion in a ratio of about 1:1. In some embodiments, the compound of Formula XI, Formula XI-1, or Formula XI-1a exists in the salt with respect to its counter ion in a ratio of about 1:2.

In some embodiments, provided here is a method of preparing a compound of Formula XI-1a from the compound of Formula VII-2a. In some embodiments, the method comprises contacting the compound of Formula VII-2a with an acid under reaction conditions sufficient to provide a compound of Formula VIa-1a, contacting the compound of Formula VIa-1a with a compound of Formula VIIIa or a salt thereof under conditions sufficient to provide the compound of Formula IX-2, or a solvate (for example, a hydrate) of each thereof, contacting the compound of Formula IX-2 with a dehydrating agent and with a base under reaction conditions sufficient, to provide the compound of Formula X-2 or a salt thereof, and contacting the compound of Formula X-2 or a salt thereof under deprotection conditions sufficient to provide the compound of Formula XI-1a or a salt thereof. In some embodiments, the process comprises purifying the compound of Formula VII-2a to a sufficient purity. In some embodiments, the purity of the compound of Formula VII-2a is about 95%. In some embodiments, the purity of the compound of Formula VII-2a is about 98%. In some embodiments, the purity of the compound of Formula VII-2a is about 99%. In some embodiments, the purity of the compound of Formula VII-2a is about 99.5%. In some embodiments, the purity of the compound of Formula VII-2a is about 99.9%. Any suitable purification method is contemplated, including but not limited to recrystallization, and chromatography.

In some embodiments, the R⁷³ is a moiety of Formula

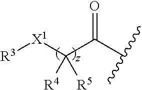

where the substituents are as defined above. Accordingly, the compound of Formula X described above has the Formula I-1.

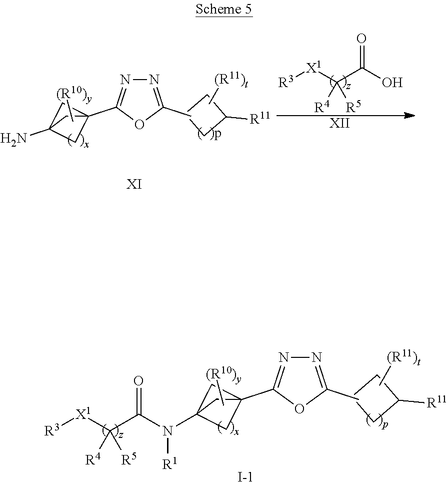

Referring to Scheme 5, in some embodiments, provided here is a method for preparing a compound of Formula I-1, or a stereoisomer or mixture of stereoisomers thereof, or a salt of each thereof, from a compound of Formula XI or a stereoisomer or mixture of stereoisomers thereof, wherein:
R³ is hydrogen, C₁₋₁₂ alkyl, C₂₋₁₂ alkenyl, C₂₋₁₂ alkynyl, C₃₋₁₀ cycloalkyl, heterocyclyl, aryl, or heteroaryl, each of which, other than hydrogen, is optionally substituted with one or more R¹¹;
each of R⁴ and R⁵ is independently hydrogen, C₁₋₁₂ alkyl, C₂₋₁₂ alkenyl, or C₂₋₁₂ alkynyl, each of which, other than hydrogen, is independently optionally substituted with one or more halo, oxo, acetyl, amino, or hydroxyl;
or R³ and R⁴, together with the atoms to which they are attached, join to form a C₃10 cycloalkyl or heterocyclyl, each of which is optionally substituted with one or more R¹¹;
or R⁴ and R⁵, together with the atoms to which they are attached, join to form a C₃10 cycloalkyl, heterocyclyl, or heteroaryl, each of which is optionally substituted with one or more R¹¹;
X¹ is O, NR⁹, or a bond;
z is 0 or 1, provided that when z is 0 and X¹ is 0, then R³ is not alkyl; and
other substituents and parameters are as defined with respect to Scheme 2.

In some embodiments, the compound of Formula XII is Formula XII-1:

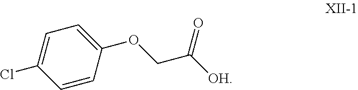 [SMILES: O=C(O)COc1ccc(Cl)cc1]

In some embodiments, if the compound of Formula XI used is a salt form, for example, the salt with a compound of Formula XII-1 acid, then no additional compound of Formula XII is needed in the reaction shown in Scheme 5.

In some embodiments, the method comprises contacting the compound of Formula XI or a stereoisomer or mixture of stereoisomers thereof with a compound of Formula XII under reaction conditions sufficient to provide the compound of Formula I-1 or a stereoisomer or mixture of stereoisomers thereof, or a salt of each thereof.

In some embodiments, X¹ is O. In some embodiments, R³ is an aryl substituted with 0-3 halo, hydroxy, or alkoxy. In some embodiments, R³ is 4-chlorophenyl.

In some embodiments, the reaction conditions providing the compound of Formula I-1 comprises:
a) optionally a first reaction step at a temperature of about 0° C. to about 5° C.;
b) optionally a second reaction step at a temperature of about 15° C. to about 30° C.; and/or
c) optionally in presence of a base and diphenylphosphinic chloride.

In some embodiments, the second reaction step is conducted at a temperature of about 20° C. to about 30° C.

In some embodiments, the reaction conditions providing the compound of Formula I-1 further comprises contacting the reaction mixture with a base, such as a carbonate, and then contacting with a mixture of isopropyl alcohol and heptane.

In some embodiments, the preparation of the compound of Formula I-1 further comprises isolating the compound of Formula XI or a stereoisomer or mixture of stereoisomers thereof, or a salt of each thereof, using heptane, IPAc, and/or IPA.

Accordingly, in some embodiments, provided here is a method for preparing the compound of Formula I-1, or a stereoisomer or mixture of stereoisomers thereof, or a salt of each thereof, comprising:
a) contacting a compound of Formula VIIa or a stereoisomer or mixture of stereoisomers thereof, with a first acid under reaction conditions sufficient to provide a compound of Formula VI, or a stereoisomer or mixture of stereoisomers thereof; and contacting the compound of Formula VI, or a stereoisomer or mixture of stereoisomers thereof, with a compound of VIII under conditions sufficient to provide a compound of Formula IX or a stereoisomer or mixture of stereoisomers thereof, or a solvate of each thereof;
b) contacting a compound of Formula VIIa or a stereoisomer or mixture of stereoisomers thereof, with the compound of VIII under conditions sufficient to provide a compound of Formula IX or a stereoisomer or mixture of stereoisomers thereof, or a solvate of each thereof;
c) contacting a compound of Formula VIIb or a stereoisomer or mixture of stereoisomers thereof, with the first acid under reaction conditions sufficient to provide a compound of Formula VI, or a stereoisomer or mixture of stereoisomers thereof; and contacting the compound of Formula VI, or a stereoisomer or mixture of stereoisomers thereof, with the compound of VIII under conditions sufficient to provide a compound of Formula IX or a stereoisomer or mixture of stereoisomers thereof, or a solvate of each thereof; or
d) contacting a compound of Formula VIIb or a stereoisomer or mixture of stereoisomers thereof with the compound of VIII under conditions sufficient to provide the compound of Formula IX or a stereoisomer or mixture of stereoisomers thereof, or a solvate of each thereof;
contacting the compound of Formula IX with a dehydrating agent and with a base under reaction conditions sufficient, to provide the compound of Formula X or a stereoisomer or mixture of stereoisomers thereof, or a salt of each thereof;
contacting the compound of Formula X, or a stereoisomer or mixture of stereoisomers thereof, or a salt of each thereof, under deprotection conditions sufficient to provide the compound of Formula XI or a stereoisomer or mixture of stereoisomers thereof, or a salt of each thereof; and
contacting the compound of Formula XI or a stereoisomer or mixture of stereoisomers thereof with a compound of Formula XII under reaction conditions sufficient to provide the compound of Formula I-1 or a stereoisomer or mixture of stereoisomers thereof, or a salt of each thereof.

In some embodiments, provided here is the compound of Formula I-1 as a racemic mixture. In some embodiments, provided here is an enantiomer of the compound of Formula I-1. In some embodiments, the compound of Formula I-1 is Formula I-1a:

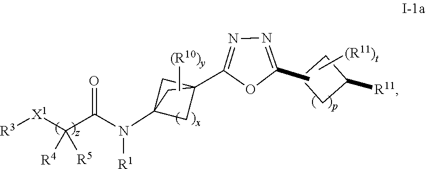

where the substituents are as defined above.

In some embodiments, provided here is a method of preparing a compound of Formula I-1a or a stereoisomer or mixture of stereoisomers thereof, or a salt of each thereof, comprising:
a) contacting a compound of Formula VII-2a or a stereoisomer or mixture of stereoisomers thereof, with a first acid under reaction conditions sufficient to provide a compound of Formula VIa-1a or a stereoisomer or mixture of stereoisomers thereof; and
contacting the compound of Formula VIa-1a, or a stereoisomer or mixture of stereoisomers thereof, with a compound of VIIIa under conditions sufficient to provide the compound of Formula IX-2 or a stereoisomer or mixture of stereoisomers thereof, or a solvate of each thereof; or
b) contacting a compound of Formula VII-2b or a stereoisomer or mixture of stereoisomers thereof with the compound of VIIIa under conditions sufficient to provide the compound of Formula IX-2 or a stereoisomer or mixture of stereoisomers thereof, or a solvate of each thereof; and
contacting the compound of Formula IX-2, or a stereoisomer or mixture of stereoisomers thereof, or a solvate of each thereof, with a dehydrating agent and a base under reaction conditions sufficient to provide the compound of Formula X-2 or a stereoisomer or mixture of stereoisomers thereof, or a salt of each thereof;
contacting the compound of Formula X-2, or a stereoisomer or mixture of stereoisomers thereof, or a salt of each thereof, under deprotection conditions sufficient to provide a compound of Formula XI-1a or a stereoisomer or mixture of stereoisomers thereof, or a salt of each thereof; and
contacting the compound of Formula XI-1a or a stereoisomer or mixture of stereoisomers thereof with a compound of Formula XII-1 under reaction conditions sufficient to provide the compound of Formula I-1a or a stereoisomer or mixture of stereoisomers thereof, or a salt of each thereof.

In some embodiments, the compound of Formula I-1a is Formula I:

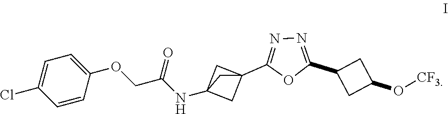

In some embodiments, provided here is a method of preparing a compound of Formula I or a salt thereof from the compound of Formula VII-2a. In some embodiments, the method comprises contacting the compound of Formula VII-2a with an acid under reaction conditions sufficient to provide a compound of Formula VIa-1a, contacting the compound of Formula VIa-1a with a compound of Formula VIIIa or a salt thereof under conditions sufficient to provide the compound of Formula IX-2, or a solvate (for example, a hydrate) of each thereof, contacting the compound of Formula IX-2 with a dehydrating agent and with a base under reaction conditions sufficient, to provide the compound of Formula X-2 or a salt thereof, contacting the compound of Formula X-2 or a salt thereof under deprotection conditions sufficient to provide the compound of Formula XI-1a or a salt thereof, and optionally contacting the compound of Formula XI-1a or a salt thereof with a compound of Formula XII-1 under reaction conditions sufficient to provide the compound of Formula I or a salt thereof. In some embodiments, the process comprises purifying the compound of Formula VII-2a to a sufficient purity. In some embodiments, the purity of the compound of Formula VII-2a is about 95%. In some embodiments, the purity of the compound of Formula VII-2a is about 98%. In some embodiments, the purity of the compound of Formula VII-2a is about 99%. In some embodiments, the purity of the compound of Formula VII-2a is about 99.5%. In some embodiments, the purity of the compound of Formula VII-2a is about 99.9%. Any suitable purification method is contemplated, including but not limited to recrystallization, and chromatography.

Any combination of steps described above may be used in the preparation of compounds described herein, including any procedures described in the Examples section.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

As described above, the compounds of this disclosure may contain one or more chiral centers. Further, the disclosure contemplates other chiral centers not explicitly described. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

### 5. SALTS AND SOLID FORMS

Further provided herein are salts of the compound of Formula VIa-1a:

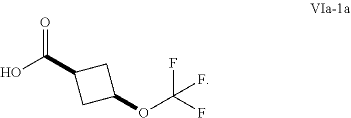

In some embodiments, the salt is selected from t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, dibenzylamine, magnesium, sodium, potassium, calcium, zinc, lithium, cesium, tetramethylammonium, and ammonium.

In some embodiments, provided herein are crystalline forms for a salt of the compound of Formula VIa-1a. In some embodiments, the solid form is a crystalline form for a salt that comprises an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a cation selected from magnesium, sodium, potassium, calcium, zinc, lithium, cesium, tetramethylammonium, and ammonium.

In some embodiments, the solid form is a crystalline form for the compound of Formula VII-2a:

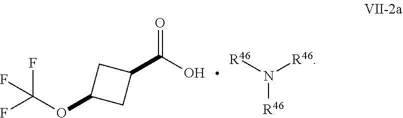

wherein each R⁴⁶ is independently hydrogen, C₁₋₆ alkyl, C₂₋₆ alkenyl, C₂₋₆ alkynyl, C₃₋₂₀ cycloalkyl, heterocyclyl, aryl, or heteroaryl, wherein each alkyl, alkenyl, alkynyl, cycloalkyl, heterocyclyl, aryl, and heteroaryl of R⁴⁶ is independently optionally substituted with one or more R¹²;
each R¹² is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³OR³¹, —OC(O)NR³OR³¹, —NR³⁰C(O)NR³OR³¹, —S(O)₁₋₂R³⁰, —S(O)₁₋₂NR³⁰, —S(O)₁₋₂NR³OR³¹, —NR³⁰S(O)₁₋₂R³¹, —NR³⁰S(O)₁₋₂NR³OR³¹, —NR³⁰C(O)R³¹, or —NR³⁰C(═O)OR³¹, wherein each alkyl, alkenyl, alkynyl, cycloalkyl, heterocyclyl, aryl, and heteroaryl of R¹² is independently optionally substituted with one or more halo or C₁₋₁₂ alkyl independently optionally substituted by one or more oxo, halo, hydroxyl, or amino; and
each R³⁰ and R³¹ is independently hydrogen or C₁₋₁₂ alkyl independently optionally substituted with one or more oxo, halo, hydroxyl, or amino; or
R³⁰ and R³¹ are taken together with the atoms to which they are attached to form heterocyclyl independently optionally substituted by one or more halo or C₁₋₁₂ alkyl independently optionally substituted by one or more oxo, halo, hydroxyl, or amino.

In some embodiments, the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

In some embodiments, the solid form is a crystalline form for the compound of Formula VII-2b:

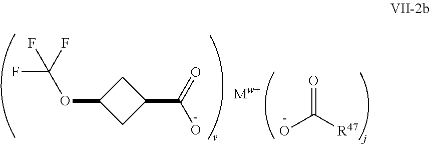

wherein w is 1, 2, or 3; v is 1 or 2; j is w-v, and is 0, 1, or 2; M^{w+} is a cation; and each R⁴⁷ is C₁₋₆ alkyl.

In some embodiments, the salt comprises a cation selected from the group consisting of magnesium, sodium, potassium, calcium, zinc, lithium, and cesium.

In some embodiments, as described above, preparing a salt form of the compound of Formula VIa-1a substantially improves the purity of the compound of Formula VIa-1a. In some embodiments, the salt is t-butylamine, L-lysine, piperazine, dicyclohexylamine, arginine, tromethamine, ethanolamine, dehydroabietylamine, or dibenzylamine. In some embodiments, the salt is t-butylamine, where the salt formation increases purity from 96.9% to 99.8% as compared to the free base starting material.

In some embodiments, provided herein is a polymorph (or crystalline) form of a t-butylamine (TBA) salt of the compound of Formula VIa-1a, which is also a crystalline form of a compound of Formula VII-1a (referred to as “Compound VIa-1a, TBA salt Form A”).

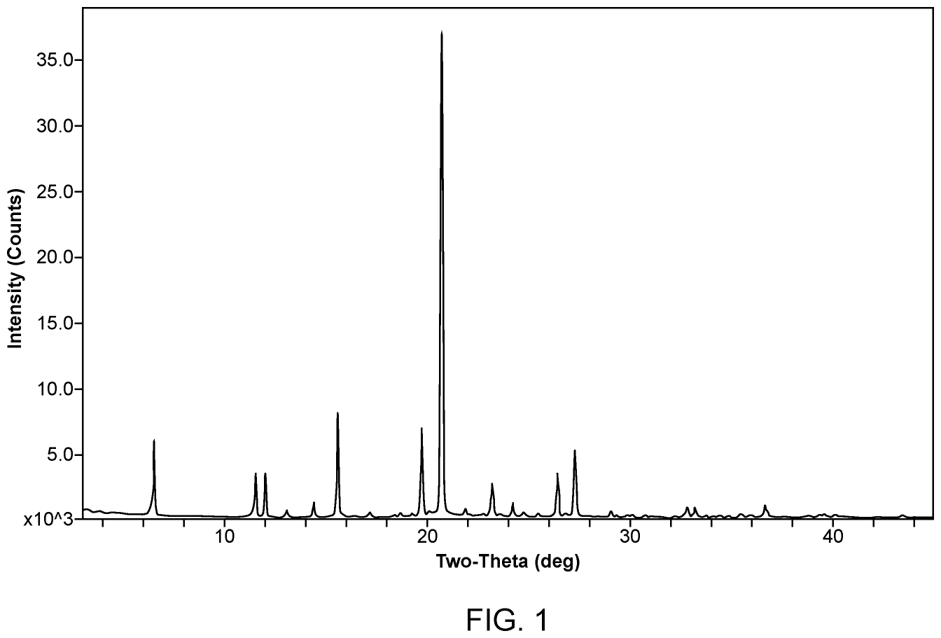

In some embodiments, Compound VIa-1a, TBA salt Form A is characterized by an X-ray powder diffraction pattern comprising one or more, or two, or three, or four, or five, or six, or seven, or eight peaks selected from 6.6, 11.6, 12.1, 15.6, 19.8, 20.8, 26.5, and 27.3 02θ±0.2° 2θ, as determined on a diffractometer using Cu-K□ radiation (λ=1.54059 Å). In some embodiments, the diffractogram further comprises one, two, three, four, five, six, seven, eight, nine, or ten additional peaks selected from 13.2, 14.4, 15.8, 17.2, 22.0, 23.2, 24.3, 32.9, 33.2, and 36.7° 2θ±0.2° 2θ. In some embodiments, compound VIa-1a, TBA salt Form A may also characterized by its full X-ray powder diffraction pattern as substantially shown in FIG. 1.

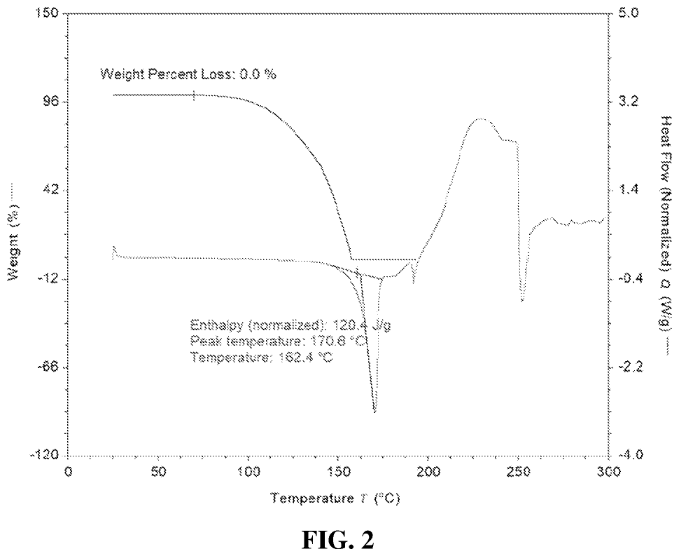

In some embodiments, Compound VIa-1a, TBA salt Form A is characterized by TGA comprising a thermogram substantially as shown in FIG. 2. In some embodiments, Compound VIa-1a, TBA salt Form A is also characterized by DSC curve comprising an endotherm at about 171° C. In another embodiment, the DSC curve is substantially as shown in FIG. 2.

In some embodiments, provided herein is a crystalline form of a dicyclohexylamine (DCHA) salt of the compound of Formula VIa-1a, which is also a crystalline form of a compound of Formula VII-1b (referred to as “Compound VIa-1a, DCHA salt Form A”).

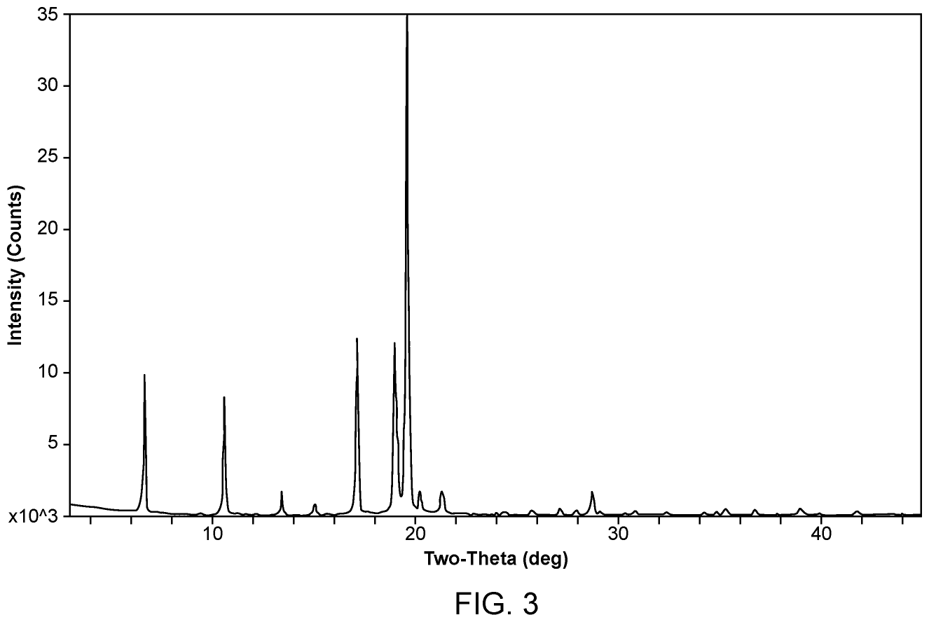

In some embodiments, Compound VIa-1a, DCHA salt Form A is characterized by an X-ray powder diffraction pattern comprising one or more, or two, or three, or four, or five peaks selected from 6.7, 10.6, 17.2, 19.0, and 19.6° 2θ±0.2° 2θ, as determined on a diffractometer using Cu-K□ radiation (λ=1.54059 Å). In some embodiments, the diffractogram further comprises one, two, three, four, five, six, seven, eight, nine, or ten additional peaks selected from 13.5, 15.1, 20.3, 21.4, 27.2, 28.0, 28.8, 35.4, 36.8, and 39.0° 2θ±0.2° 2θ. In some embodiments, compound VIa-1a, DCHA salt Form A may also characterized by its full X-ray powder diffraction pattern as substantially shown in FIG. 3.

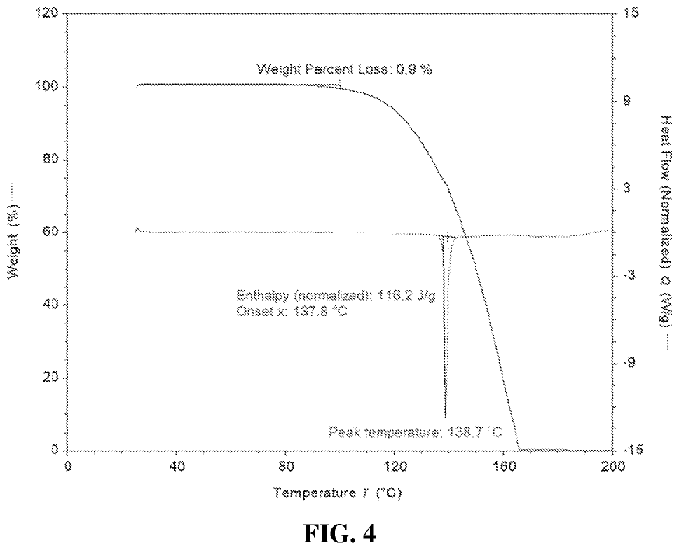

In some embodiments, Compound VIa-1a, DCHA salt Form A is characterized by TGA comprising a thermogram substantially as shown in FIG. 4. In some embodiments, Compound VIa-1a, DCHA salt Form A is also characterized by DSC curve comprising an endotherm at about 139° C. In another embodiment, the DSC curve is substantially as shown in FIG. 4.

In some embodiments, provided herein is a crystalline form of an L-arginine salt of the compound of Formula VIa-1a, which is also a crystalline form of a compound of Formula VII-1c (referred to as “Compound VIa-1a, L-arginine salt Form A”).

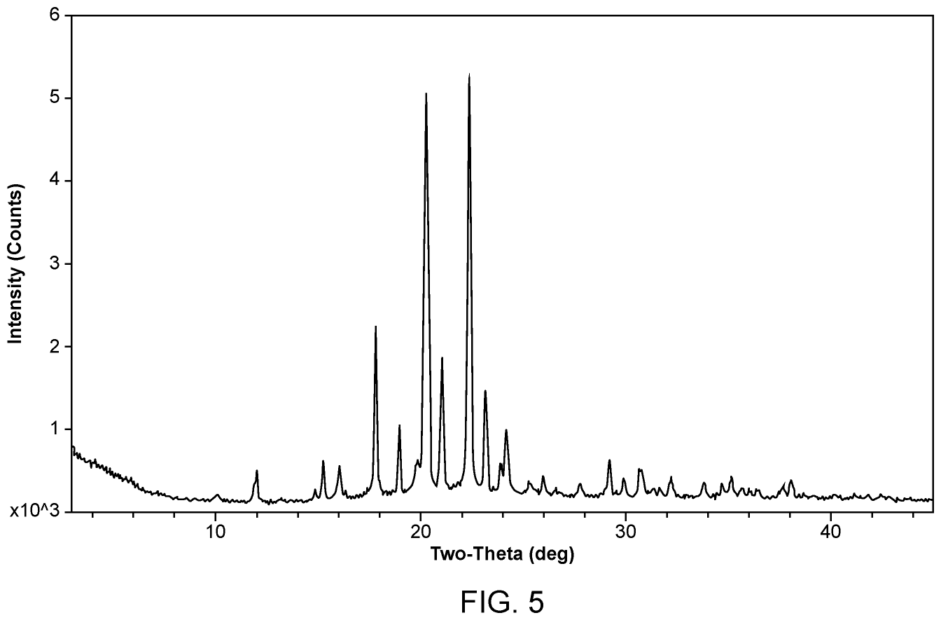

In some embodiments, Compound VIa-1a, L-arginine salt Form A is characterized by an X-ray powder diffraction pattern comprising one or more, or two, or three, or four, or five, or six, or seven peaks selected from 17.8, 19.0, 20.3, 21.1, 22.4, 23.2, and 24.2° 2θ±0.2° 2θ, as determined on a diffractometer using Cu-K□ radiation (λ=1.54059 Å). In some embodiments, the diffractogram further comprises one, two, three, four, five, six, seven, eight, nine, or ten additional peaks selected from 12.0, 15.3, 16.1, 19.9, 23.9, 26.0, 29.2, 30.7, 32.2, and 38.0° 2θ±0.2° 2θ. In some embodiments, the diffractogram further comprises one, two, three, four, five, six, or seven additional peaks selected from 14.9, 27.8, 29.9, 33.8, 34.7, 35.2, and 37.7° 2θ±0.2° 2θ. In some embodiments, Compound VIa-1a, L-arginine salt Form A may also characterized by its full X-ray powder diffraction pattern as substantially shown in FIG. 5.

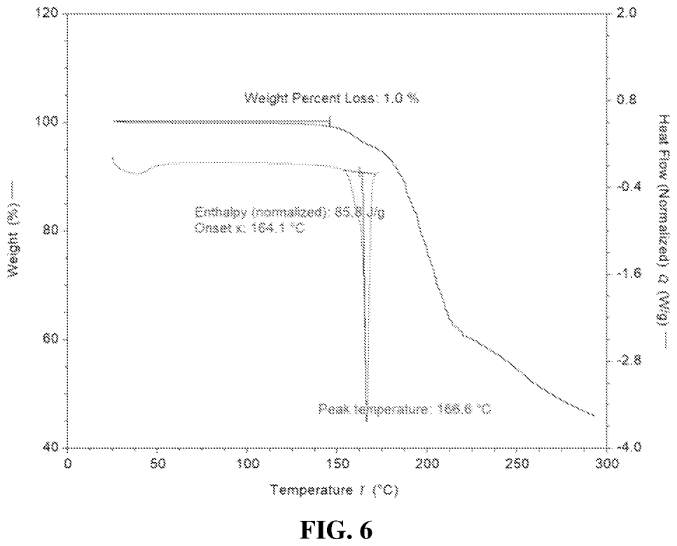

In some embodiments, Compound VIa-1a, L-arginine salt Form A is also characterized by TGA comprising a thermogram substantially as shown in FIG. 6. In some embodiments, Compound VIa-1a, L-arginine salt Form A is also characterized by DSC curve comprising an endotherm at about 167° C. In another embodiment, the DSC curve is substantially as shown in FIG. 6.

In some embodiments, provided herein is a crystalline form of a tromethamine (TMA) salt of the compound of Formula VIa-1a, which is also a crystalline form of a compound of Formula VII-1d (referred to as “Compound VIa-1a, TMA salt Form A”).

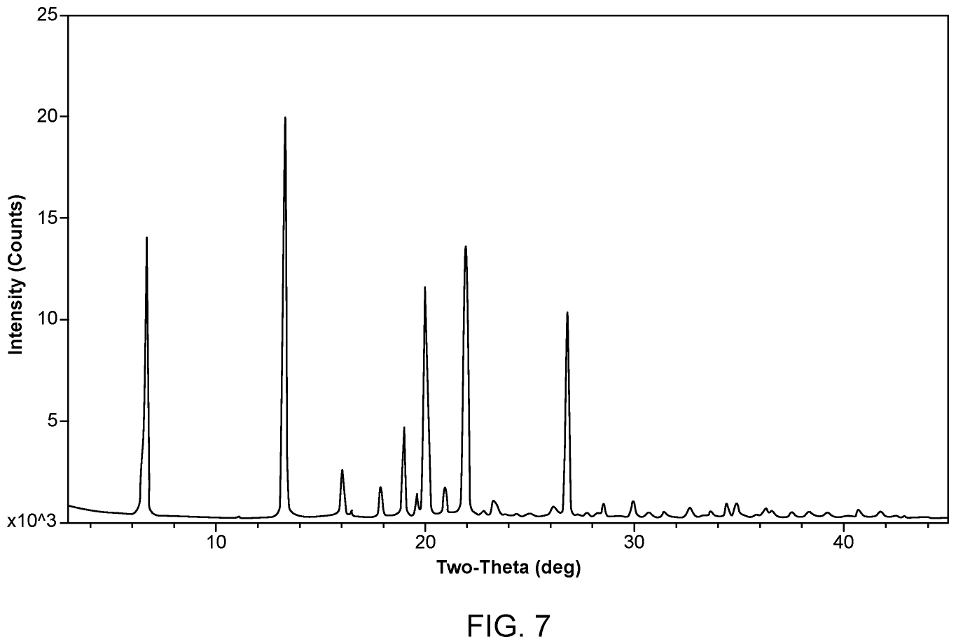

In some embodiments, Compound VIa-1a, TMA salt Form A is characterized by an X-ray powder diffraction pattern comprising one or more, or two, or three, or four, or five, or six peaks selected from 6.6, 13.2, 19.9, 20.1, 21.8, and 26.7° 2θ±0.2° 2θ, as determined on a diffractometer using Cu-K□ radiation (λ=1.54059 Å). In some embodiments, the diffractogram further comprises one, two, three, four, five, six, seven, eight, nine, ten, or eleven additional peaks selected from 15.9, 17.8, 18.9, 19.5, 20.9, 22.5, 28.4, 29.8, 33.5, 34.3, and 34.8° 2θ±0.2° 2θ. In some embodiments, Compound VIa-1a, TMA salt Form A may also characterized by its full X-ray powder diffraction pattern as substantially shown in FIG. 7.

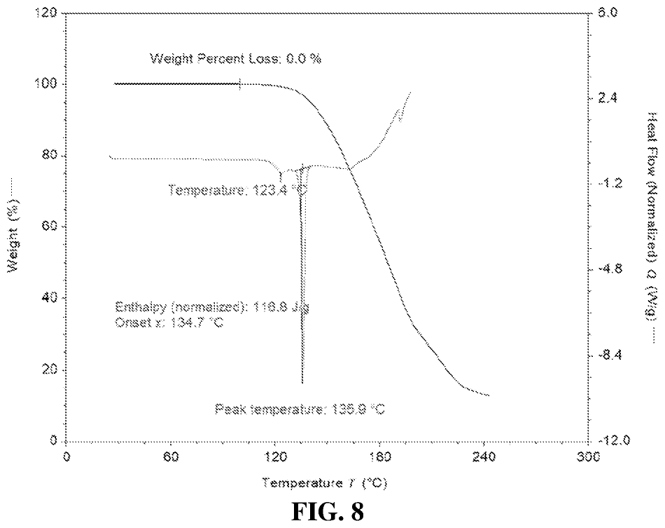

In some embodiments, Compound VIa-1a, TMA salt Form A is also characterized by TGA comprising a thermogram substantially as shown in FIG. 8. In some embodiments, Compound VIa-1a, TMA salt Form A is also characterized by DSC curve comprising endotherms at about 123° C. and at about 136° C. In another embodiment, the DSC curve is substantially as shown in FIG. 8.

Further provided herein is compound of Formula XI-1a:

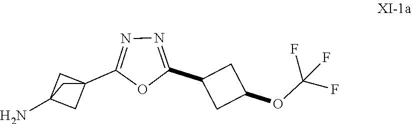

and solid forms, and salts of each thereof.

In some embodiments, provided herein is a crystalline form of a 4-chlorophenoxyacetic acid salt of the compound of Formula XI-1a (also referred to as “Compound XI-1a, 4-chlorophenoxyacetic acid salt Form A”).

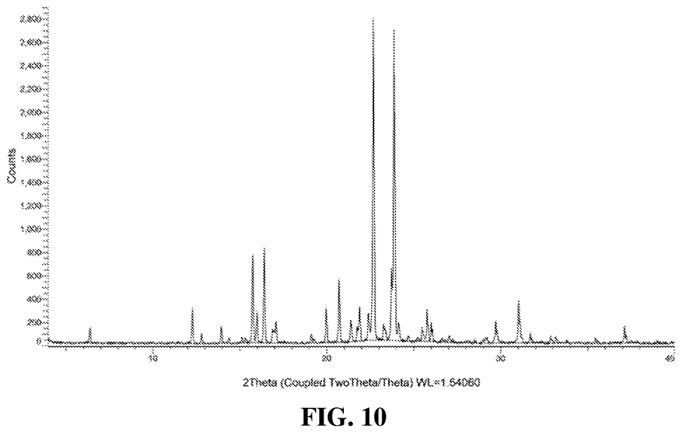

In some embodiments, Compound XI-1a, 4-chlorophenoxyacetic acid salt Form A is characterized by an X-ray powder diffraction pattern comprising one or more, or two, or three, or four, peaks selected from 15.7, 16.4, 22.7, and 23.8° 2θ±0.2° 2θ, as determined on a diffractometer using Cu-K□ radiation (λ=1.54059 Å). In some embodiments, the diffractogram further comprises one, two, three, four, or five, additional peaks selected from 20.0, 20.7, 21.9, 25.8, and 31.0° 2θ±0.2° 2θ. In some embodiments, Compound XI-1a, 4-chlorophenoxyacetic acid salt Form A may also characterized by its full X-ray powder diffraction pattern as substantially shown in FIG. 10.

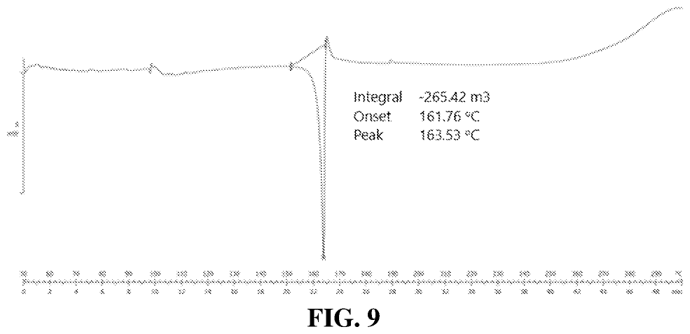

In some embodiments, Compound XI-1a, 4-chlorophenoxyacetic acid salt Form A is also characterized by DSC curve comprising endotherms at about 162° C. (onset). In another embodiment, the DSC curve is substantially as shown in FIG. 9.

In some embodiments, provided herein is a crystalline form of the compound of Formula XI-1a free base.

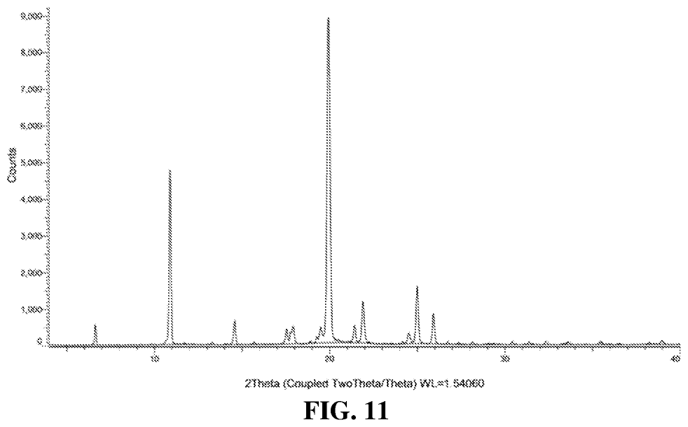

In some embodiments, Compound XI-1a, free base Form A is characterized by an X-ray powder diffraction pattern comprising one or more, or two, or three, or four, or five, or six, or seven peaks selected from 6.6, 10.9, 14.5, 19.9, 21.9, 25.0, 25.9° 2θ±0.2° 2θ, as determined on a diffractometer using Cu-K□ radiation (λ=1.54059 Å). In some embodiments, crystalline Compound XI-1a, free base may also characterized by its full X-ray powder diffraction pattern as substantially shown in FIG. 11.

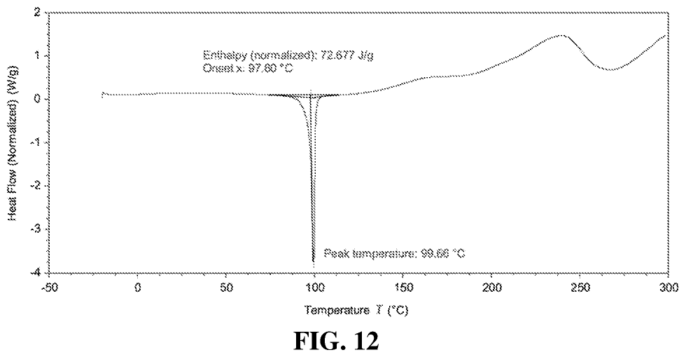

In some embodiments, Compound XI-1a, free base Form A is also characterized by DSC curve comprising endotherms at about 98° C. (onset). In another embodiment, the DSC curve is substantially as shown in FIG. 12.

In some embodiments, provided herein is a crystalline form of a dioxalate salt of the compound of Formula XI-1a, (also referred to as “Compound XI-1a, dioxalate salt Form A”).

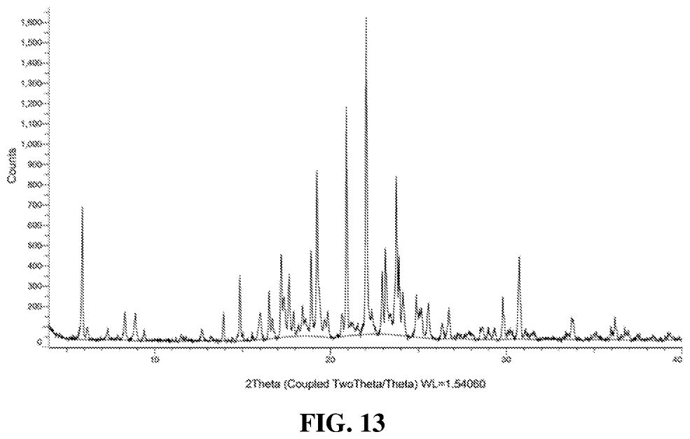

In some embodiments, Compound XI-1a, dioxalate salt Form A is characterized by an X-ray powder diffraction pattern comprising one or more, or two, or three, or four, or five peaks selected from 5.9, 19.2, 20.9, 22.0, and 23.7° 2θ±0.2° 2θ, as determined on a diffractometer using Cu-K□ radiation (λ=1.54059 Å). In some embodiments, the diffractogram further comprises one, two, three, four, or five, additional peaks selected from 14.9, 18.9, 23.1, 23.9, and 30.7° 2θ±0.2° 2θ. In some embodiments, Compound XI-1a, dioxalate salt Form A may also characterized by its full X-ray powder diffraction pattern as substantially shown in FIG. 13.

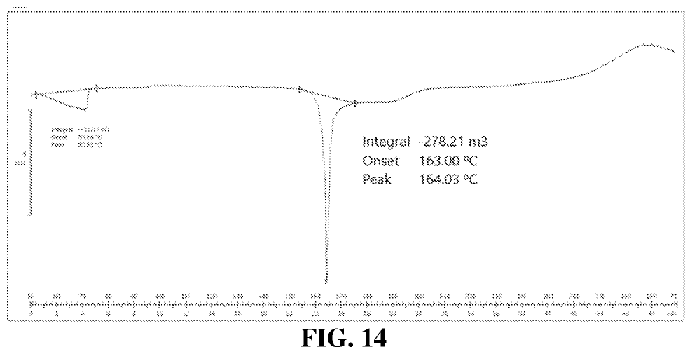

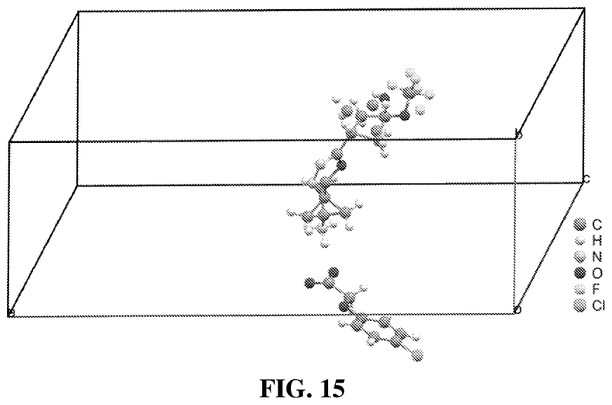

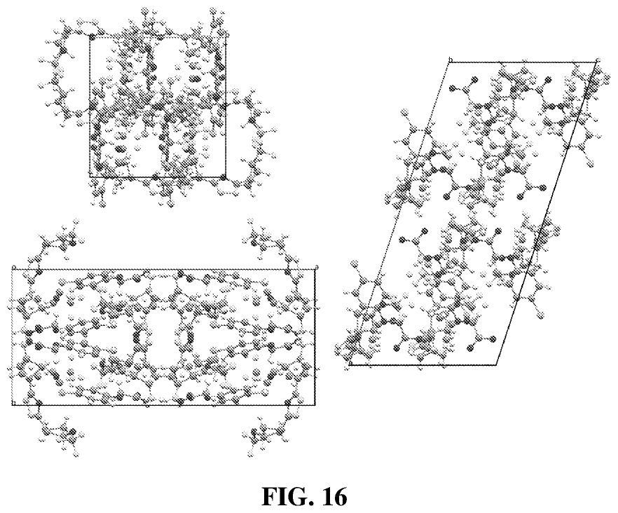

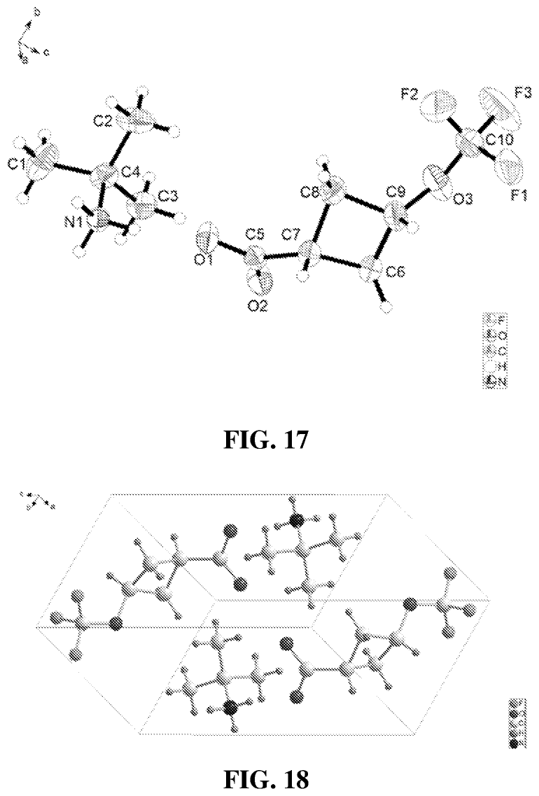

In some embodiments, Compound XI-1a, dioxalate salt Form A is also characterized by DSC curve comprising endotherms at about 163.0° C. (onset). In another embodiment, the DSC curve is substantially as shown in FIG. 14.

The present disclosure therefore provides the following numbered embodiments:

Embodiment 1. A method of preparing a compound of Formula V-2:

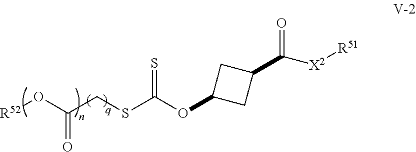

or a salt thereof, comprising contacting a compound of Formula IIa-1:

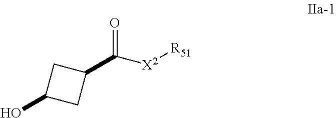

with an activating agent and then contacting with a compound of Formula IV:

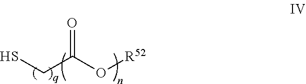

under reaction conditions sufficient to provide the compound of Formula V-2, wherein:
n is 0 or 1;
q is 1, 2, 3, 4, 5, 6, 7, 8, 9, 10, 11, or 12;
X² is O or NR¹;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided the compound is not ethyl cis-3-[(methylthio)thioxomethoxy]cyclobutanecarboxylate.

Embodiment 2. The method of Embodiment 1, wherein X² is O.

Embodiment 3. The method of Embodiment 1, wherein X² is NR⁵³.

Embodiment 4. The method of Embodiment 1, wherein X² is NR⁵³, and R⁵¹ and R⁵³ together with the nitrogen atom join to form a heterocyclyl.

Embodiment 5. The method of Embodiment 4, wherein R⁵¹ and R⁵³ together with the nitrogen atom join to form a -morpholino.

Embodiment 6. The method of Embodiment 1, wherein n is 0.

Embodiment 7. The method of Embodiment 1, wherein R⁵² is C₁₋₁₂ alkyl.

Embodiment 8. The method of any one of Embodiments 1-7, wherein the activating reagent is selected from the group consisting of 1,1′-thiocarbonyldiimidazole, thiram, 1,1′-thiocarbonyldi-2(1H)-pyridone, 1,1′-(thiocarbonyl)bis-1H-benzotriazole, and di-2-pyridyl thionocarbonate.

Embodiment 9. The method of Embodiment 8, wherein the activating reagent is 1,1′-thiocarbonyldiimidazole.

Embodiment 10. The method of any one of Embodiments 1-9, wherein the contacting of the compound of Formula IIa-1 with the activating agent comprises:
a) optionally a reaction temperature of about 0° C. to about 60° C.; and/or
b) optionally a reaction time duration of about 3 hours to about 72 hours.

Embodiment 11. The method of any one of Embodiments 1-10, wherein the contacting with the compound of Formula IV comprises contacting under reaction conditions that comprise:
a) optionally a reaction temperature of about 20° C. to about 80° C.; and/or
b) optionally a reaction time duration of about 1 hour to about 48 hours.

Embodiment 12. A compound selected from the group consisting of:

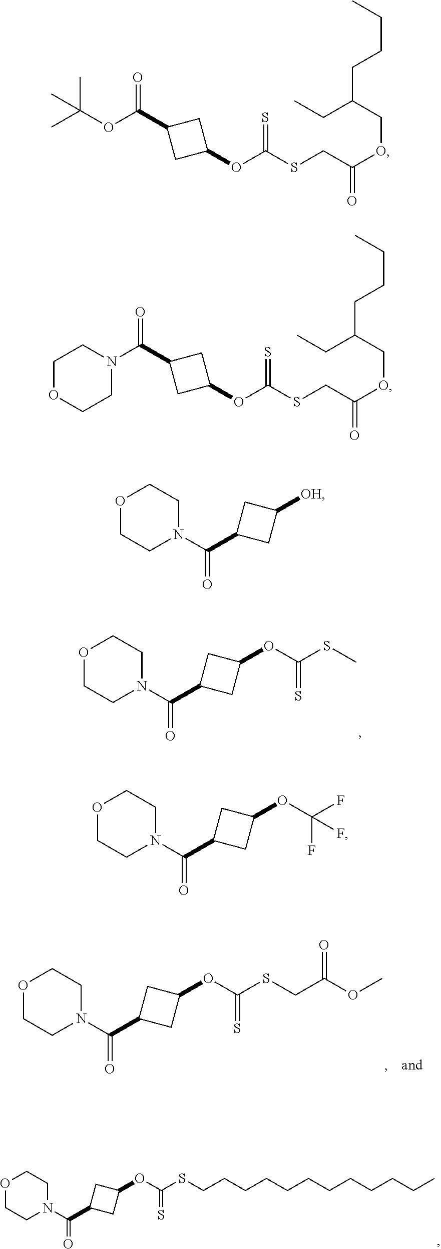

or a salt of each thereof.

Embodiment 13. A salt of a compound of Formula VIa-1a:

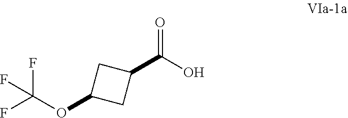

wherein the salt comprises an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a cation selected from magnesium, sodium, potassium, calcium, zinc, lithium, cesium, tetramethylammonium, and ammonium.

Embodiment 14. A method for preparing a salt of a compound of Formula VIa-1a:

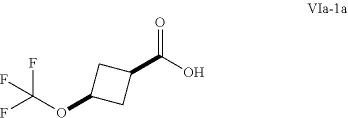

comprising contacting a compound of Formula V-2:

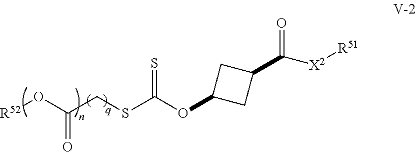

or a salt thereof, with an electrophilic bromine source and a fluorinating agent to provide the compound of Formula VIa-1a; and
contacting the compound of Formula VIa-1a with an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a base containing a cation selected from magnesium, sodium, potassium, calcium, zinc, lithium, cesium, tetramethylammonium, and ammonium; under reaction conditions sufficient to provide the compound of Formula V-2,
   wherein:
n is 0 or 1;
q is 1, 2, 3, 4, 5, 6, 7, 8, 9, 10, 11, or 12;
X² is O or NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided that when R⁵¹ is C₁₋₁₂ alkyl or silyl, or when X² is NR⁵³, the method comprises a hydrolysis step prior to forming the salt.

Embodiment 15. The method of Embodiment 14, wherein the electrophilic bromine source comprises DBDMH (1,3-dibromo-5,5-dimethylhydantoin) or NBS (n-bromosuccinimide).

Embodiment 16. The method of Embodiment 14, wherein X² is O.

Embodiment 17. The method of Embodiment 14, wherein R⁵¹ is hydrogen or C₁₋₁₂ alkyl.

Embodiment 18. The method of Embodiment 14, wherein X² is NR⁵³.

Embodiment 19. A method for preparing a salt of a compound of Formula VIa-1a:

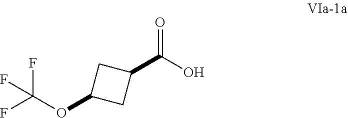

comprising contacting a compound of Formula V-2:

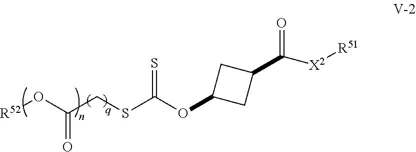

or a salt thereof, with an electrophilic bromine source and a fluorinating agent, followed by hydrolysis to form VIa-1a; and
contacting VIa-1a with an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a base containing a cation selected from magnesium, sodium, potassium, calcium, zinc, lithium, cesium, tetramethylammonium, and ammonium; under reaction conditions sufficient to provide the salt of VIa-1a:
n is 0 or 1;
q is 1, 2, 3, 4, 5, 6, 7, 8, 9, 10, 11, or 12;
X² is NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided the compound is not ethyl cis-3-[(methylthio)thioxomethoxy]cyclobutanecarboxylate.

Embodiment 20. The salt of Embodiment 13, having a Formula of VII-2a:

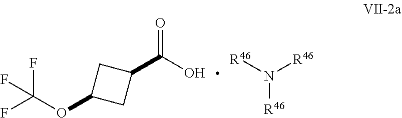

wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

Embodiment 21. The salt of Embodiment 13, having a Formula VII-1a:

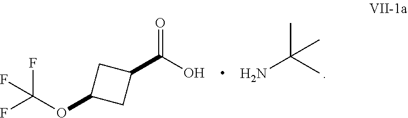

Embodiment 22. The salt of Embodiment 13, having a Formula VII-1b

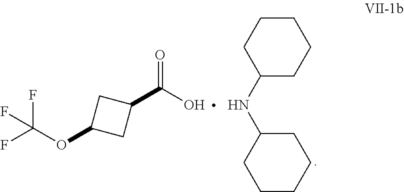 [SMILES: O=C(O)[C@H]1C[C@@H](OC(F)(F)F)C1 ; C1CCC(NC2CCCCC2)CC1]

Embodiment 23. The salt of Embodiment 13, having a Formula VII-1c:

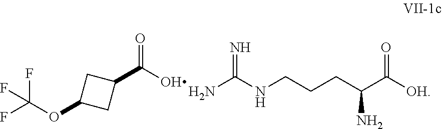 [SMILES: O=C(O)[C@H]1C[C@@H](OC(F)(F)F)C1 ; N=C(N)NCCC[C@H](N)C(=O)O]

Embodiment 24. The salt of Embodiment 13, having a Formula VII-1d:

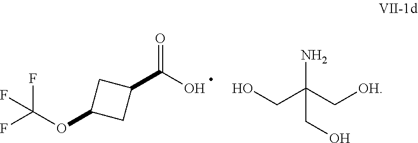 [SMILES: O=C(O)[C@H]1C[C@@H](OC(F)(F)F)C1 ; NC(CO)(CO)CO]

Embodiment 25. Form A polymorph of a t-butylamine (TBA) salt of (1s,3s)-3-(trifluoromethoxy)cyclobutane-1-carboxylic acid (Compound VIa-1a, TBA salt Form A), that exhibits an X-ray powder diffraction pattern having one or more peaks selected from 6.6, 11.6, 12.1, 15.6, 19.8, 20.8, 26.5, and 27.3° 2q±0.2° 2q, wherein the X-ray powder diffraction pattern is made using Cu-Kat radiation.

Embodiment 26. The Compound VIa-1a, TBA salt Form A polymorph of Embodiment 25, further characterized by:
i) the X-ray powder diffraction pattern further comprising one or more peaks selected from 13.2, 14.4, 15.8, 17.2, 22.0, 23.2, 24.3, 32.9, 33.2, and 36.7° 2q±0.2° 2q;
ii) a diffractogram substantially as shown in FIG. 1;
iii) a differential scanning calorimetry (DSC) comprising an endotherm at about 171° C.; or
iv) a thermogravimetric analysis (TGA) comprising a thermogram substantially as shown in FIG. 2.

Embodiment 27. Form A polymorph of a dicyclohexylamine (DCHA) salt of (1s,3s)-3-(trifluoromethoxy)cyclobutane-1-carboxylic acid (Compound VIa-1a, DCHA salt Form A), that exhibits an X-ray powder diffraction pattern having one or more peaks selected from 6.7, 10.6, 17.2, 19.0, and 19.6° 2q±0.2° 2q, wherein the X-ray powder diffraction pattern is made using Cu-Kat radiation.

Embodiment 28. The Compound VIa-1a, DCHA salt Form A polymorph of Embodiment 27, further characterized by:
i) the X-ray powder diffraction pattern further comprising one or more peaks selected from 13.5, 15.1, 20.3, 21.4, 27.2, 28.0, 28.8, 35.4, 36.8, and 39.0° 2q±0.2° 2q;
ii) a diffractogram substantially as shown in FIG. 3;
iii) a differential scanning calorimetry (DSC) comprising an endotherm at about 139° C.; or
iv) a thermogravimetric analysis (TGA) comprising a thermogram substantially as shown in FIG. 4.

Embodiment 29. Form A polymorph of an L-arginine salt of (1s,3s)-3-(trifluoromethoxy)cyclobutane-1-carboxylic acid (Compound VIa-1a, L-arginine salt Form A), that exhibits an X-ray powder diffraction pattern having one or more peaks selected from 17.8, 19.0, 20.3, 21.1, 22.4, 23.2, and 24.2° 2q±0.2° 2q, wherein the X-ray powder diffraction pattern is made using Cu-Kα radiation.

Embodiment 30. The Compound VIa-1a, L-arginine salt Form A polymorph of Embodiment 29, further characterized by:
i) the X-ray powder diffraction pattern further comprising one or more peaks selected from 12.0, 15.3, 16.1, 19.9, 23.9, 26.0, 29.2, 30.7, 32.2, and 38.0° 2q±0.2° 2q;
ii) a diffractogram substantially as shown in FIG. 5;
iii) a differential scanning calorimetry (DSC) comprising an endotherm at about 167° C.; or
iv) a thermogravimetric analysis (TGA) comprising a thermogram substantially as shown in FIG. 6.

Embodiment 31. Form A polymorph of a tromethamine (TMA) salt of (1s,3s)-3-(trifluoromethoxy)cyclobutane-1-carboxylic acid (Compound VIa-1a, TMA salt Form A), that exhibits an X-ray powder diffraction pattern having one or more peaks selected from 6.6, 13.2, 19.9, 20.1, 21.8, and 26.7° 2q±0.2° 2q, wherein the X-ray powder diffraction pattern is made using Cu-Kα radiation.

Embodiment 32. The Compound VIa-1a, TMA salt Form A polymorph of Embodiment 31, further characterized by:
i) the X-ray powder diffraction pattern further comprising one or more peaks selected from 15.9, 17.8, 18.9, 19.5, 20.9, 22.5, 28.4, 29.8, 33.5, 34.3, and 34.8° 2q±0.2° 2q;
ii) a diffractogram substantially as shown in FIG. 7;
iii) a differential scanning calorimetry (DSC) comprising an endotherm at about 123° C. and about 136° C.; or
iv) a thermogravimetric analysis (TGA) comprising a thermogram substantially as shown in FIG. 8.

Embodiment 33. A method of preparing a compound of Formula IX-2:

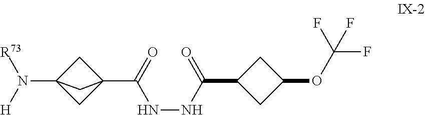

or a solvate thereof, comprising:
contacting a salt of a compound of Formula VIa-1a:

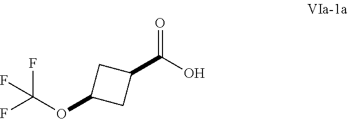

with an acid under reaction conditions sufficient to provide the compound of Formula VIa-1a; and
contacting the compound of Formula VIa-1a with a compound of Formula VIIIa or a salt

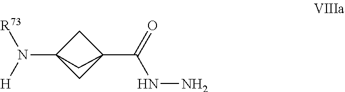

under conditions sufficient to provide the compound of Formula IX-2 or a solvate thereof,
wherein R⁷³ is hydrogen or a protecting group.

Embodiment 34. The method of Embodiment 33, wherein the protecting group is tert-butoxycarbonyl or of Formula

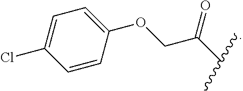 [SMILES: *C(=O)COc1ccc(Cl)cc1]

Embodiment 35. The method of Embodiment 33 or 34, wherein the salt is Formula VII-2a:

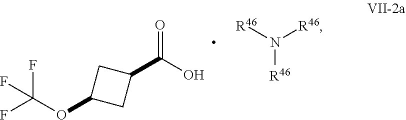

wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

Embodiment 36. The method of Embodiment 34 or 35, wherein the compound of Formula IX-2 is prepared by contacting the compound of Formula V-2 with a fluorinating agent and DBDMH (1,3-dibromo-5,5-dimethylhydantoin) or NBS (n-bromosuccinimide).

Embodiment 37. The method of Embodiment 36, wherein the fluorinating agent is HF-pyridine.

Embodiment 38. A method of preparing a compound of Formula X-2:

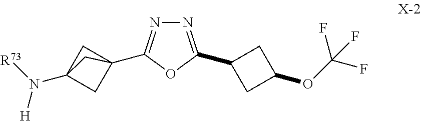

or a salt thereof, comprising:
contacting a salt of a compound of Formula VIa-1a:

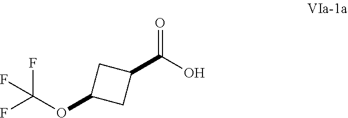 [SMILES: O=C(O)[C@H]1C[C@@H](OC(F)(F)F)C1]

with a first acid under reaction conditions sufficient to provide the compound of Formula VIa-1a; and
contacting the compound of Formula VIa-1a with a compound of Formula VIIIa or a salt

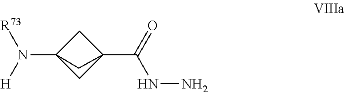

under conditions sufficient to provide a compound of Formula IX-2:

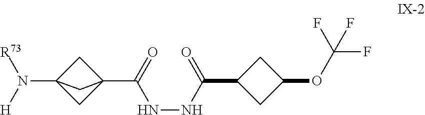

or a solvate thereof; and
contacting the compound of Formula IX-2 or a solvate thereof with a dehydrating agent and with a base under reaction conditions sufficient, to provide the compound of Formula X-2 or a salt thereof, wherein R⁷³ is hydrogen or a protecting group.

Embodiment 39. The method of Embodiment 38, wherein the salt of the compound of Formula VIa-1a is Formula VII-2a:

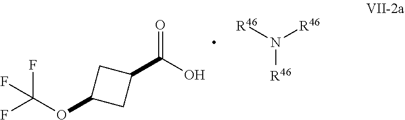

wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

Embodiment 40. The method of Embodiment 38 or 39, wherein R⁷³ is a protecting group.

Embodiment 41. The method of any one of Embodiments 38-40, wherein R⁷³ is tert-butoxycarbonyl.

Embodiment 42. The method of any one of Embodiments 38-40, wherein the protecting group is tert-butoxycarbonyl or of Formula

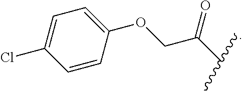 [SMILES: *C(=O)COc1ccc(Cl)cc1]

Embodiment 43. A method of preparing a compound of Formula XI-1a:

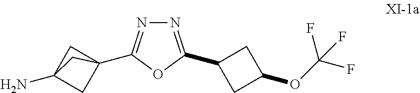

or a salt thereof, comprising:
contacting a salt of a compound of Formula VIa-1a:

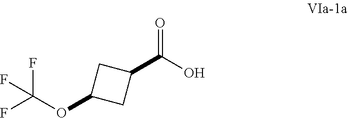 [SMILES: O=C(O)[C@H]1C[C@@H](OC(F)(F)F)C1]

with a first acid under reaction conditions sufficient to provide the compound of Formula VIa-1a; and
contacting the compound of Formula VIa-1a with a compound of Formula VIIIa or a salt thereof:

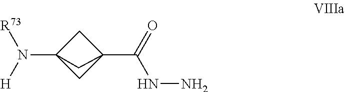

under conditions sufficient to provide a compound of Formula IX-2:

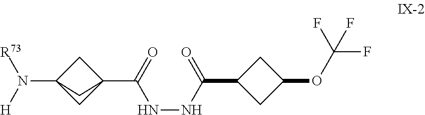

or a solvate thereof;
contacting the compound of Formula IX-2 or a solvate thereof with a dehydrating agent and with a base under reaction conditions sufficient, to provide a compound of Formula X-2:

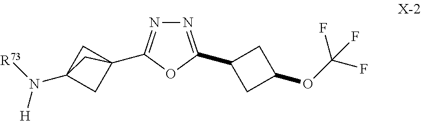

or a salt thereof; and
contacting the compound of Formula X-2 or a salt thereof, under deprotection conditions sufficient to provide the compound of Formula XI-1a or a salt thereof, wherein R⁷³ is hydrogen or a protecting group.

Embodiment 44. The method of Embodiment 43, wherein the protecting group is tert-butoxycarbonyl or of Formula

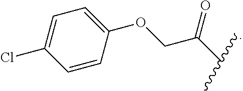 [SMILES: *C(=O)COc1ccc(Cl)cc1]

Embodiment 45. The method of Embodiment 43 or 44, wherein the salt of the compound of Formula VIa-1a of Formula VII-2a:

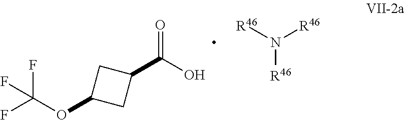

wherein the N(R⁴⁶)³ moiety is selected from the group consisting of t-butylamine, L-lysine, piperazine, dicyclohexylamine, arginine, tromethamine, ethanolamine, dehydroabietylamine, and dibenzylamine.

Embodiment 46. A method of preparing a compound of Formula I:

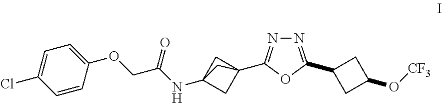

or a salt thereof, comprising:
contacting a salt of a compound of Formula VIa-1a:

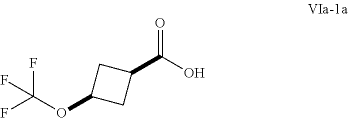

with a first acid under reaction conditions sufficient to provide the compound of Formula VIa-1a; and
contacting the compound of Formula VIa-1a with a compound of Formula VIIIa or a salt thereof:

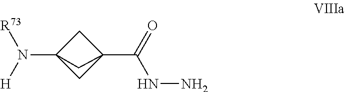

under conditions sufficient to provide a compound of Formula IX-2:

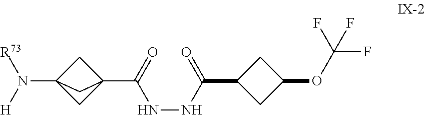

or a solvate thereof;
contacting the compound of Formula IX-2 or a solvate thereof with a dehydrating agent and with a base under reaction conditions sufficient, to provide a compound of Formula X-2:

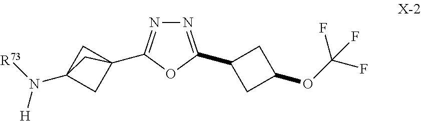

or a salt thereof; and
contacting the compound of Formula X-2 or a salt thereof, under deprotection conditions sufficient to provide the compound of Formula XI-1a:

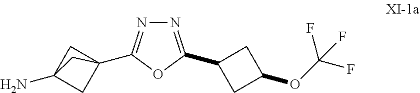

or a salt thereof, and
optionally contacting the compound of Formula XI-1a or a salt thereof with a compound of Formula XII-1:

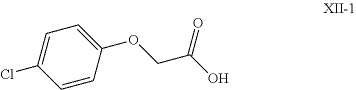 [SMILES: O=C(O)COc1ccc(Cl)cc1]

under reaction conditions sufficient to provide the compound of Formula I or a salt thereof, wherein R⁷³ is hydrogen or a protecting group.

Embodiment 47. The method of Embodiment 46, wherein the salt of the compound of Formula VIa-1a is Formula VII-2a:

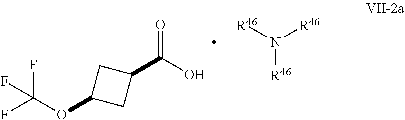

wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

Embodiment 48. The method of Embodiment 46 or 47, wherein the salt of the compound of Formula X-2 is a hydrochloride salt, oxalic acid salt, and 4-chlorophenoxyacetic acid salt.

Embodiment 49. The method of any of Embodiments 46-48, wherein the deprotection conditions comprises contacting the compound of Formula X-2 with a second acid.

Embodiment 50. The method of Embodiment 49, wherein the second acid is generated in situ from an acid precursor in the presence of a protic compound, optionally at a temperature of about 10° C. to about 100° C.

Embodiment 51. The method of any of Embodiments 46-50, wherein the deprotection conditions comprise generating hydrogen chloride in situ.

Embodiment 52. The method of any of Embodiments 46-51, further comprising isolating the compound of Formula XI-1a or a salt thereof using heptane, IPAc, and/or IPA.

Embodiment 53. The method of Embodiment 46, wherein the reaction conditions providing the compound of Formula I comprises:
a) a temperature of about 0° C. to about 5° C., and warming to a temperature of about 20° C. to about 25° C.; and/or
b) optionally in presence of a base and diphenylphosphinic chloride.

Embodiment 54. The method of Embodiment 53, wherein the reaction conditions providing the compound of Formula I further comprises contacting a reaction mixture of Embodiment 53 with a base and further contacting with a mixture of isopropyl alcohol and heptane.

Embodiment 55. The method of Embodiment 38, 46, or 47, wherein the dehydrating agent is selected from the group consisting of sulfonyl chloride, sulfonic anhydride, phosphonic anhydride, carbonic acid anhydride bearing electron withdrawing groups, and a mixture of triphenylphosphine with carbon tetrachloride.

Embodiment 56. The method of Embodiment 55, wherein the dehydrating agent is selected from the group consisting of 4-toluenesulfonyl chloride, methanesulfonyl chloride, toluenesulfonic anhydride, methanesulfonyl anhydride, tri-n-propylphosphonic anhydride, trifluoroacetic anhydride, and the mixture of triphenylphosphine with carbon tetrachloride.

Embodiment 57. The method of any of Embodiments 35-56, wherein the solvate is a polar aprotic solvent.

Embodiment 58. The method of any of Embodiments 35-56, wherein the solvate is hydrate.

Embodiment 59. The method of Embodiment 49, wherein the polar aprotic solvent is selected from the group consisting of N,N-dimethylformamide, N-methylpyrrolidone, and dimethylacetamide.

Embodiment 60. The method of any of Embodiments 38-59, wherein the base is N,N-diisopropylethylamine (DIPEA), triethylamine (TEA), or potassium carbonate.

Embodiment 61. The method of any of Embodiments 38-60, wherein the reaction conditions sufficient to provide the compound of Formula X-2 or a salt thereof comprises acetonitrile as a solvent.

Embodiment 62. The method of any of Embodiments 43-61, preparing a salt of the compound of Formula XI-1a, wherein the salt of the compound of Formula XI-1a is a hydrochloride salt, oxalic acid salt, or a 4-chlorophenoxyacetic acid salt.

Embodiment 63. The method of Embodiment 62, wherein the salt of the compound of Formula XI-1a is 4-chlorophenoxyacetic acid salt.

Embodiment 64. A hydrate of a compound of Formula IX-1a:

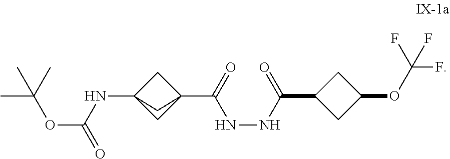

Embodiment 65. The hydrate of Embodiment 64, wherein a stoichiometric ratio of the compound of Formula IX-1a in the hydrate to water is about 1:1.

Embodiment 66. A dioxalate salt of 3-(5-((1s,3s)-3-(trifluoromethoxy)cyclobutyl)-1,3,4-oxadiazol-2-yl)bicyclo[1.1.1]pentan-1-amine:

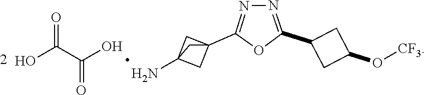

Embodiment 67. A 4-chlorophenoxyacetic acid salt of 3-(5-((1s,3s)-3-(trifluoromethoxy)cyclobutyl)-1,3,4-oxadiazol-2-yl)bicyclo[1.1.1]pentan-1-amine:

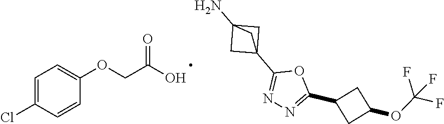

Embodiment 68. Crystalline 3-(5-((1s,3s)-3-(trifluoromethoxy)cyclobutyl)-1,3,4-oxadiazol-2-yl)bicyclo[1.1]pentan-1-amine (Compound XI-1a) free base Form A:

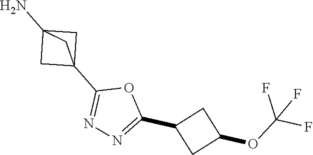

characterized by an X-ray powder diffraction pattern comprising one or more, or two, or three, or four, or five, or six, or seven peaks selected from 6.6, 10.9, 14.5, 19.9, 21.9, 25.0, 25.9° 2θ±0.2° 2θ, as determined on a diffractometer using Cu-Kα radiation (λ=1.54059 Å).

Embodiment 69. The Compound XI-1a free base Form A of Embodiment 68, further characterized by DSC curve comprising endotherms at about 98° C. (onset).

### EXAMPLES

### X-Ray Powder Diffraction (XRPD)

Standard XRPD patterns were collected using a Bruker D8 Advance diffractometer (Bruker, GER). The X-ray source is a copper (Cu) tube that was operated at 40 kV and 40 mA. Powder samples were prepared on zero-background silicon (Si) holders using manual light pressure to keep the sample surfaces flat. Each sample was analyzed from 3 to 45° 2θ with an effective step size of 0.02° 2θ and 0.08 s exposure time.

In situ high temperature XRPD patterns were collected using a Malvern PANalytical Aeris diffractometer (Malvern Panalytical, UK) coupled with a BTS500 online hot stage (Anton Paar, AT). The X-ray source was operated at 40 kV and 7.5 mA, and powder samples were prepared on zero-background Si holders. Each sample was analyzed from 8 to 45° 2θ with an effective step size of 0.02° 2θ. The measurement time for each sample is 20 min.

### Thermogravimetry Analysis (TGA)

Thermogravimetric analysis (TGA) was performed on a TA Instruments Discovery 550 (TA, US). Each sample was placed into a pre-tared platinum pan and heated from 25° C. to target temperature at a heating rate of 10° C./min under a nitrogen atmosphere. The nitrogen purge was 40 mL/min at the balance and 60 mL/min at the furnace.

### Differential Scanning Calorimetry (DSC)

DSC analyses were conducted on a TA Instruments Discovery 250 (TA, US). Calibration of the instrument temperature and cell constant was performed using indium. The DSC cell was kept under a nitrogen purge of 50 mL/min during each analysis. The sample was placed in a Tzero hermetic pan with a pinhole and was heated from 25° C. to the target temperature at a rate of 10° C./min. Several DSC analyses was conducted by using Tzero hermetic pan without a pinhole, since the sample may sublime at high temperature.

### Dynamic Vapor Sorption (DVS) Analysis

DVS analysis was carried out using a Surface Measurement System DVS Intrinsic analyzer (SMS, UK). The instrument balance was calibrated with standard weights. Approximately 15-20 mg of sample was loaded into a pan for analysis. The sample was analyzed at 25° C. in 10% relative humidity (RH) steps from 50% to 95% RH (adsorption cycle), from 95% to 0% RH (desorption cycle), and from 0% to 50% RH (adsorption cycle). The movement from one step to the next occurred either after satisfying the equilibrium criterion of 0.002% weight change (dm/dt) or, if the equilibrium criterion was not met, after six hours.

### Polarized Light Microscopy (PLM)

Microscopic images of crystals were captured on a Nikon Ci-POL445 Polarizing Microscope (Nikon, Japan) under a suitable objective.

### Gas Chromatography (GC)

Shimadzu GC-2104 (Shimadzu, Japan) was utilized for chemical purity characterization. Detailed chromatographic conditions are listed in Table 1.

TABLE 1

| Injection | Injection mode | Split mode | | |
|---|---|---|---|---|
| | Split ratio | 10:01 | | |
| | Carrier gas | He | | |
| | Temperature | 280° C. | | |
| Column | Column | HP-5 (30 m, 0.32 mm ID, Agilent Technologies) | | |
| | Flow mode | constant linear velocity | | |
| | Flow rate | 1 mL/min | | |
| | Injection volume | 1 μL | | |
| Oven | Heating rate (° C./min) | N/A | 2 | 15 |
| | Temperature (° C.) | 80 | 120 | 250 |
| | Time(min) | 2 | 0 | 0 |

| | | |
|---|---|---|
| Detector | Temperature | 300° C. |
| | Detector | FID |

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemie or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Preparation of (1s,3s)-3-hydroxycyclobutane-1-carboxylic acid (II-1)

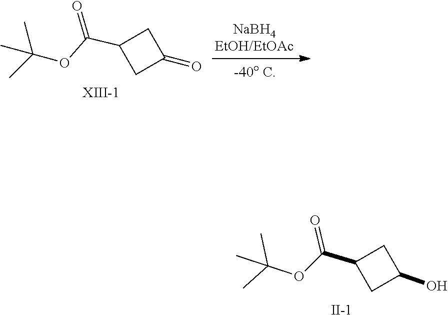

Ethylacetate (EtOAc) (277.2 kg) and EtOH (120.0 kg) were charged into a 1000 L Stainless Steel (SS) reactor at about 15° C.˜25° C. Sodium borohydride (NaBH₄, 9.36 kg, 247 mol, 0.35 eq) was added into the reactor under nitrogen at about 15° C.˜25° C. The 1000 L reactor was cooled to about −35° C.˜−45° C. EtOAc (68.4 kg) and 3-oxo-cyclobutanecarboxylic acid tert-butyl ester (XIII-1) (120.0 kg, 705 mol, 1.0 eq) were charged into a 500 L vessel. The solution was stirred for about 0.5 h at about 10° C.˜30° C. The XIII-1 solution in the 500 L vessel was charged into the NaBH₄ 1000 L reactor at about −35° C.˜−45° C. The reaction mixture was stirred at about −35° C.˜−45° C. for about 3 h until the reaction was deemed complete.

Deionized water (336.0 kg) and ammonium chloride (24.0 kg) were added into a 2000 L glass-lined (GL) reactor. The solution was stirred at about 10° C.˜30° C. The ammonium chloride solution in the 2000 L GL reactor was cooled to about −3° C.˜5° C. The reaction mixture in the 1000 L SS reactor was charged into the 2000 L GL reactor. During the addition, the temperature was controlled in the range of about −3° C.˜5° C. to control the exotherm. The reaction mixture of 2000 L GL reactor was heated to about 5° C.˜15° C. and agitated for about 6 h. The reaction mixture in 2000 L GL reactor was filtered at about 5° C.˜15° C., and then the cake was washed with EtOAc (54.0 kg). The filtrate was transferred to the 1000 L GL reactor at about 5° C.˜15° C., settled, and phase-separated. The aqueous phase and EtOAc (216 kg) were added into the 1000 L GL reactor at about 5° C.˜15° C., stirred for about 1 h, and settled for about 1 h. The aqueous phase was discarded. The combined organic phases were washed with water (120 kg) once at about 5° C.˜15° C., and the aqueous layer was discarded. The organic solution in the 1000 L GL reactor was concentrated at gradually increasing temperature under a pressure (P) less than or equal to about −0.8 mPa, until the temperature reaches about 60° C. Heptane (81.6 kg) was added into the 1000 L GL reactor, concentrated under P≤−0.08 mPa, until the temperature reaches about 60° C. Then, another portion of heptane (81.6 kg) was added into the 1000 L GL reactor, concentrated under P≤−0.08 mPa, until the temperature reaches about 60° C. The removal of EtOAc and EtOH was confirmed. Heptane (120 kg) was added into the solution, and heated to about 50° C.˜60° C., to dissolve the solids. The solution in the 1000 L GL reactor was cooled to about 8° C.˜12° C. and stirred for about 1 h at about 10° C.˜15° C. The mixture was centrifuged in a centrifuge at about 8° C.˜12° C. The centrifugal solids were washed with heptane (81.6 kg), and the solids were weighed. Heptane (96.8 kg) and centrifugal solid were charged into the 1000 L GL reactor, heated to about 50-60° C. until the solid was dissolved completely. The solution in the 1000 L GL reactor was cooled to about 8° C.˜12° C. and stirred for about 1 h at about 8° C.˜12° C. The product was isolated by centrifuge at about 8° C.˜12° C., washed with heptane (66.0 kg), and dried at about 35° C.˜40° C. to give II-1. Yield: 83.38 kg (68.7% yield).

¹H NMR (DMSO-d₆, 400 MHz) δ=5.14 (d, J=6.8 Hz, 1H), 3.88-3.97 (m, 1H), 2.32-2.42 (m, 3H), 1.88-1.91 (m, 2H), 1.39 (s, 9H) ppm.

### Example 2: Preparation of tert-butyl (1s,3s)-3-((((2-((2-ethylhexyl)oxy)-2-oxoethyl)thio)carbonothioyl)oxy)cyclobutane-1-carboxylate (V-1a)

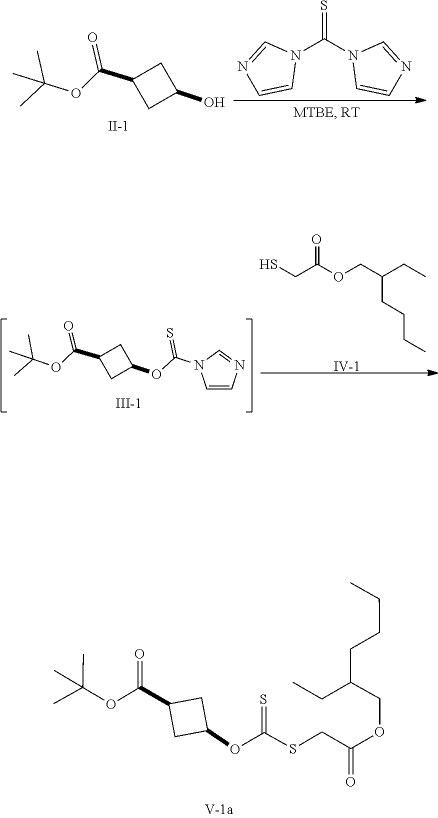

Methyl tert-butyl ether (MTBE) (310.8 kg) was charged into a 1000 L GL reactor and stirred for 0.5 h at about 15˜30° C. II-1 (42 kg, 244 mol, 1.0 eq) was charged into the 1000 L GL reactor and stirred for about 0.5 h at about 15˜30° C. Thiocarbonyldiimidazole (47.88 kg, 269 mol, 1.1 eq) was charged into the 1000 L GL reactor at about 20˜25° C. and stirred at about 20˜25° C. for 4 h until the reaction was deemed complete.

2-ethylhexyl thioglycolate (55 kg, 269 mol, 1.1 eq) was charged into the 1000 L GL reactor at about 20˜25° C. and heated to about 40˜45° C. for about 2 h until the reaction was deemed complete. The reaction mixture was then cooled to about 10˜20° C. Deionized (DI) water (420.0 kg) was charged into the 1000 L GL reactor at about 10˜20° C., stirred for about 1 h, and settled for about 4 h. The layers were separated, and the aqueous was extracted with MTBE (155.4 kg) at about 10˜20° C. The combined organic phases were charged into the 1000 L GL reactor at about 10˜20° C. with 5% by weight sodium bicarbonate solution (420 kg), agitated for about 1 h, and allowed to settle for about 4 h. The layers were separated. Anhydrous sodium sulfate (63 kg) was charged into the 1000 L GL reactor at about 10˜20° C., stirred for about 12 h and sampled to confirm water content to be less than about 2%. The reaction mixture was filtered at about 10˜20° C. and the filter cake was rinsed with MTBE (63 kg). The filtrate was charged to a 1000 L GL reactor at about 15˜30° C. The organic solution was concentrated under P≤−0.8 mPa at gradually increasing temperature, until the temperature reaches about 50° C. Dichloromethane (DCM) (223.44 kg) was charged into the 1000 L GL reactor and concentrated under P≤−0.08 mPa at gradually increasing temperature, until the temperature reaches about 40° C. The distillation process of DCM was repeated until the DCM content was less than or equal to about 5.0%, the MTBE content was less than or equal to about 2.0%, each by percentage of the integrated area under the Gas Chromatography curve, and the water content was less than or equal to about 500 ppm (by Karl Fischer method). The mixture was cooled to 15˜30° C. The liquid was used as-is for the next step.

¹H NMR (CDCl₃, 400 MHz) δ=5.43-5.36 (m, 1H), 4.11-4.05 (m, 2H), 3.92 (s, 2H), 2.75-2.63 (m, 3H), 2.48-2.37 (m, 2H), 1.68-1.56 (m, 1H), 1.45 (s, 9H), 1.39-1.25 (m, 8H), 0.92-0.87 (m, 6H) ppm.

### Example 3: Preparation of tert-butyl (1s,3s)-3-(dithiocarboxyoxy)cyclobutane-1-carboxylate (III-1b)

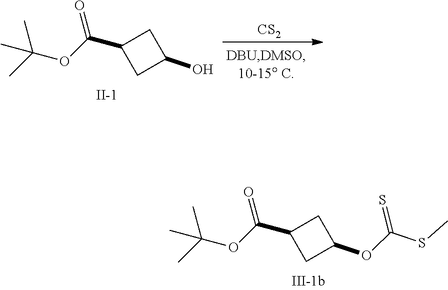

Compound II-1 (9.67 kg, 56.2 mol, 1.0 eq) was dissolved in DMSO (29 L) at 25° C. to give a colorless solution. The mixture was cooled to 10° C. within 0.5 hour. DBU (9.40 kg, 61.8 mol, 1.10 eq) was added into the reaction mixture drop-wise over 0.5 hours to keep the inner temperature between 10-15° C. The reaction mixture was stirred at 10° C. for 0.5 hour. CS₂ (6.41 kg, 84.2 mol, 1.50 eq) was added drop-wise into the reaction mixture over 1 hour to keep the inner temperature between 10-15° C., and the solution turned from colorless to yellow. The reaction mixture was stirred at 10° C. for 0.5 hour. Mel (8.77 kg, 61.8 mol, 1.10 eq) was drop-wise added into the reaction mixture over 1.5 hour to keep the inner temperature between 10-15° C. The mixture was warmed to 25° C. within 0.5 hour and stirred for 12 hours. HPLC_IPC (Rt-SM=2.226, Rt-product=3.918) showed that the starting material was consumed completely. The reaction mixture was cooled to 10° C. 10% NaH₂PO₄ (29 L) was drop-wise added into the reaction mixture over 2 hours to keep the inner temperature of between 10-15° C. The mixture was extracted with n-heptane (20 L×3). The combined organic layer was washed with water (10 L×2) and brine (10 L×2). The organic layer was concentrated in vacuo at 45° C. to give a yellow solution (30 L). Active carbon (3.0 kg) was added into the solution in one portion at 20° C. The mixture was stirred at 20° C. for 12 hours. The mixture was filtered through a pad of Celite at 25° C. The filter cake was washed with n-heptane (10 L). The eluent turn to colorless. The filtrate was concentrated in vacuo at 45° C. to provide compound III-1b (5.1 kg, 19.2 mol, 66.6% yield, 98.5% purity). ¹H NMR (d6-DMSO, 400 MHz) δ=5.37-5.44 (m, 1H), 2.76-2.78 (m, 1H), 2.65-2.68 (m, 2H), 2.55 (s, 3H), 2.23-2.26 (m, 2H), 1.40 (s, 9H) ppm.

The title compound can be used as an alternative to compound III-1 in the preparation of compound V-1a as in Example 2, above.

### Example 4: Preparation of (1s,3s)-3-(trifluoromethoxy)cyclobutane-1-carboxylic acid (VIa-1a)

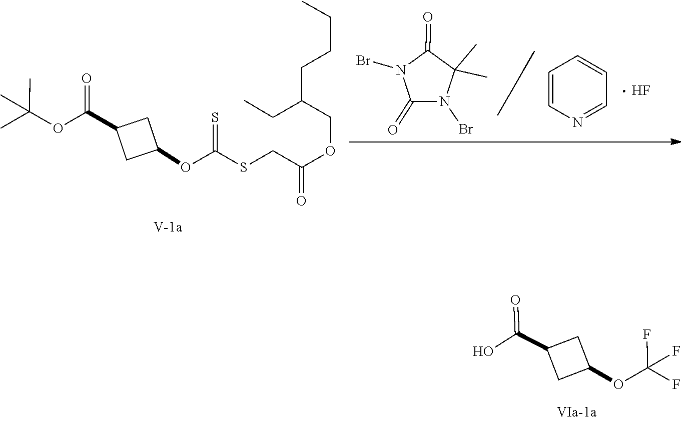

DCM (235 kg) and 1,3-dibromo-5,5-dimethylhydantoin (DBDMH) (75.2 kg, 263 mol, 3.5 eq) were charged into 500 L perfluoroalkoxy alkane (PFA) coated reactor at about 15˜30° C., and the vessel was cooled to about −75° C.˜−50° C. Hydrogen fluoride pyridine (130.2 kg, 1.31 kmol, 61 eq) was charged into the vessel via dropping tank at about −75° C.˜−50° C., and then the tank was rinsed with about 10˜20 kg DCM. After the addition was complete, the mixture was aged at the same temperature for about 1 h. Separately, V-1a (38.2 kg, 78 mol, 1.0 eq) and DCM (18.3 kg) was mixed and transferred into the vessel via dropping tank maintaining about −75° C.˜−50° C. at about 10˜30 kg/h addition rate. Upon completion of the addition, the reaction mixture was warmed to about 15° C.˜25° C., and aged for about 2 h until the reaction was deemed complete. To a separate 1000 L PTFE reactor at about 15° C.˜30° C., water (286.5 kg) and ice (95.5 kg) were added. While controlling the temperature to about −5° C.˜15° C., the reaction mixture was transferred to the ice-water mixture via pump and stirred for 4 h. The mixture was further stirred for an additional about 2 h at about 0° C.˜20° C. and then the phases were separated. The aqueous phase was extracted with DCM (191 kg) three times. The combined organic phases were washed with water (355.3 kg) two times. The organic layer was transferred to a GL reactor and distilled at a temperature less than or equal to about 50° C. until no distillate was observed, and the contents were cooled to about 20° C.˜30° C. Water (68.8 kg) and potassium carbonate (22.9 kg) were separately mixed, and then combined with the DCM product mixture at not more than about 30° C. The mixture was agitated for about 2 h and then the layers were separated. The aqueous phase was washed with DCM (38 kg) three times. At about 0˜30° C., HCl was added dropwise to adjust the pH to about 1˜2. The phases separated and the aqueous layer was extracted with DCM (68.8 kg) one time. The DCM product stream was transferred to a 1000 L GL reactor and concentrated at not more than about 50° C., P≤-0.08 MPa until no fractions were observed. After isolation by vacuum distillation up to about 110° C.,P≤5 mmHg, VIa-1a was received as an oil. Three batches were combined for the vacuum distillation to give a yield of 99.1 kg (based on assay, 55% yield based on input assay of V-1a). ¹H NMR (DMSO-d₆, 400 MHz) δ=12.42 (br s, 1H), 4.78-4.70 (m, 1H), 2.74-2.65 (m, 1H), 2.59-2.56 (m, 2H), 2.28-2.25 (m, 2H) ppm. ¹⁹F NMR (DMSO-d₆, 400 MHz) δ=−57.9 ppm.

### Example 5. Alternative Preparation of (1s,3s)-3-(trifluoromethoxy)cyclobutane-1-carboxylic acid (VIa-1a)

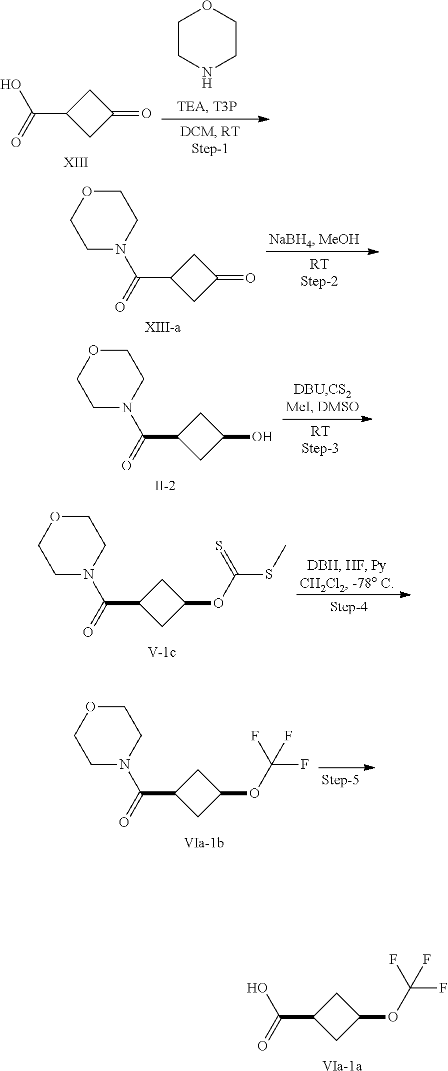

Step-1: To a stirred solution of 3-oxo cyclobutane carboxylic acid (XIII) (300 g, 1 equiv) in anhydrous DCM (3 L, 10 vol) was added T3P (1.5 equiv, 50% solution in EtOAc) followed by morpholine (1.1 equiv) at 0° C. under inert atmosphere and reaction mixture was then allowed to stir at RT for 30 min. The reaction mass was then cooled 0° C., and TEA (3 equiv) was added. The reaction mixture was then allowed to stir at RT for 8 h. Upon completion of the reaction as monitored by TLC, the reaction mass was quenched with water (1.5 L). Layers were separated, and organic layer was washed with 10% NaHCO₃ solution (1×1 L). The aqueous layer was extracted in DCM (3×2 L). The combined organic layers were dried over anhydrous Na₂SO₄, and solvent was evaporated to dryness to afford product (2) as yellow thick liquid, solidified upon standing at −20° C. in refrigerator. The crude product was taken to next without further purification (410 g, 85% yield). Melting point: 36.0°-45.0° C. ¹H NMR (CDCl₃, 400 MHz) δ=3.70-3.62 (m, 6H), 3.55-3.47 (m, 4H), 3.36-3.28 (m, 1H), 3.24-3.15 (m, 2H) ppm.

Step-2: To a stirred solution of XIII-a (100 g, 1 equiv) in anhydrous methanol (70 vol) was added NaBH₄ (0.5 equiv) in portions (over a period of 60 min) at 0° C. under inert atmosphere. Reaction mass was then allowed to stir at RT for 2 h. At this stage, the progress of the reaction was monitored by TLC. Reaction mixture was quenched with ice-water (1 vol), and then solvent was evaporated to dryness. Residue obtained was diluted water (2 vol) and acidified to pH=1 to 2 with 1.5N solution of HCl in water at 0° C. and allowed to stir at RT for 1 h. Extracted in 15 to 20% methanol in dichloromethane (5×2 L). The combined extract was dried (Na₂SO₄), and solvent was evaporated to dryness to afford crude II-2 as pale-yellow gum slowly solidified upon standing (96 g, 95% yield). The crude product obtained was predominantly cis-isomer (>96%). Melting point: 86.1°-89.1° C. ¹H NMR (CDCl₃, 400 MHz) δ=4.20 (p, J=7.2 Hz, 1H), 3.67-3.62 (m, 4H), 3.60-3.45 (m, 4H), 2.79-2.70 (m, 1H), 2.60-2.53 (m, 2H), 2.27-2.18 (m, 2H) ppm.

Step-3: Compound II-2 (50 g, 1 equiv) was dissolved in anhydrous DMSO (75 mL, 1.5 vol) and added DBU (1.1 equiv) at 0° C. and allowed to stir at RT for 1 h under inert atmosphere. To the reaction mixture was then added carbon disulfide (1.5 equiv) in drops at 0° C., and allowed to stir at RT for 2 h. Methyl iodide (1.1 equiv) was then added to the reaction mixture at 0° C., and allowed the reaction to stir at RT for 2 h. Reaction mass was treated with 10% aqueous NaH₂PO₄ (500 mL) and EtOAc (1000 mL). Organic layer was separated and washed with water (2×250 mL). Aqueous layer was extracted again in EtOAc (2×200 mL). The combined extract was dried, and solvent was removed under vacuum. Crude product V-1c obtained was slowly solidified as orange brown upon standing at −20° C. for overnight period (63 g, 85% yield). Melting point: 50.5°-60.1° C. ¹H NMR (CDCl₃, 400 MHz) δ=5.51 (p, 1H, J=7.2 Hz), 3.70-3.64 (m, 6H), 3.42-3.40 (m, 2H), 2.92-2.86 (m, 1H), 2.78-2.72 (m, 2H), 2.64-2.58 (m, 5H) ppm.

Step-4: To a solution of DBH (3 equiv) in DCM (1250 mL, 25 vol) at −78° C. in a polyethene bottle was added HF-pyridine (22 equiv) under open air and allowed to stir at the same temperature for 30 min. Then a solution of V-1c (50 g, 1 equiv) in DCM (250 mL, 5 vol) was added dropwise at −78° C. (the reaction mass turned to reddish brown) and reaction mass was allowed to stir at the same temperature for 30 min. Reaction mass was then allowed to stir at RT for 6 h. The progress of the reaction was monitored by TLC & HPLC. Reaction mixture was poured slowly onto a mixture of NaHSO₃ (10 vol, 10% solution), NaOH (40 vol, 10% solution) and NaHCO₃ (200 g), such that the pH was approx. 10 to 11. Layers were separated, and aqueous layer was further extracted in dichloromethane (2×1000 mL). The combined extract was washed with 1.5N HCl solution (750 mL), water (2×750 mL), and solvent was evaporated to dryness to afford the crude product. Crude product VIa-1b was triturated with petroleum ether to remove the non-polar impurities (35 g, 76% yield). Melting point: 45.8°-56.9° C. ¹H NMR (CDCl₃, 300 MHz)=4.58 (p, J=7.6 Hz, 1H), 3.67-3.60 (m, 4H), 3.51 (bs, 4H), 2.83-2.73 (m, 1H), 2.59-2.53 (m, 4H) ppm.

Step-5: Method-1: 4N HCl in dioxane (50 mL) was added drop-wise to VIa-1b (5 g, 1 equiv) at 0° C., followed by water (10 mL). The reaction mass was heated to 100° C. for 5 h. The progress of the reaction was monitored by TLC. TLC showed complete reaction. Solvent was concentrated under vacuum and the reaction mass was diluted with water (10 mL). Aqueous layer was basified with 5% aqueous NaOH solution (pH ˜10 to 11) and washed with MTBE (3×50 mL) to remove the non-polar impurities. The reaction mass was acidified with 1.5N HCl (pH ˜1 to 2) and the aqueous layer was further extracted in EtOAc (3×75 mL). The combined extract was dried over anhydrous sodium sulfate, and solvent was evaporated to dryness to afford the product VI (2.9 g, 80% yield).

Step-5: Method-2: Compound VIa-1b (10 g, 1 equiv) was suspended in conc. HCl (375 mL, 37.5 vol) at RT. The reaction mixture was heated to 120° C. for 4 h. At this stage TLC showed complete reaction. Reaction mass was cooled to RT, diluted with water (100 mL). Extracted in EtOAc (3×250 mL). The combined extract was concentrated in vacuum. Residue obtained was suspended in water (50 mL) and basified to pH=10 to 12 using 5% NaOH solution at 0° C. Aqueous layer was washed with MTBE (4×100 mL). Aqueous layer was cooled to 0° C., and acidified to pH=1 to 2, and extracted in EtOAc (3×150 mL). Solvent was dried (Na₂SO₄) and concentrated under vacuum to afford the acid to afford the product VI (4.7 g, 64% yield).

### Example 6: Preparation of methyl 2-((((1s,3s)-3-(morpholine-4-carbonyl)cyclobutoxy)carbonothioyl)thio)acetate

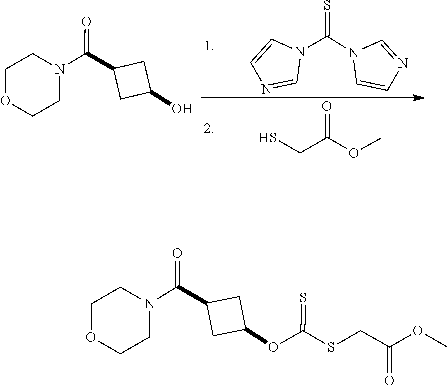

To a stirred mixture of (3-hydroxycyclobutyl)-morpholino-methanone (1225 g, 6.7 mol, 1.0 equiv) in DMF (1.85 L, 1.5 V) at 0-5° C. was added 1,1′-thiocarbonyldiimidazole (TCDI) (1424 g, 8.0 mol, 1.2 equiv). The reaction mixture was stirred at this temperature for 30 min before being warmed to 20-30° C. and stirred for 4 h. Subsequently, methyl thioglycolate (711 g, 6.7 mol, 1.0 equiv) was charged at 20-30° C., and the reaction mass was stirred at this temperature for 2 h. The reaction mixture was quenched with ice cold water (12.4 L, 10.0 V) and stirred for 30 min. The resulting precipitate was filtered and the solid was washed with water (1.2 L, 1.0 V). This solid was added to MTBE (3.7 L, 3.0 V), and stirred at 40-45° C. for 1 h before being cooled to 20-25° C. and stirred for 30 min. The mass was then filtered, and the filter cake was washed with MTBE (1.2 L, 1.0 V) before being dried in vacuo at 40-50° C. A mixture of crude product in a solution of 60% methanol in water (3.7 L, 3.0 V) was heated at 55-60° C. for 1 h. The mass was then cooled to 20-30° C. and stirred for approx. 30 min before being cooled to 0-5° C. and stirred for 1 h. Then the mixture was filtered and the solid washed with a cooled solution of 60% methanol in water (1.2 L, 1.0 V) before being dried in vacuo at 45-50° C. Yield: 1320 g (63%). Mass spectrum: Calculated MH+=334.08, observed=334.19. ¹H NMR (CDCl₃, 400 MHz) δ=5.45 (p, J=7.2 Hz, 1H), 3.91 (s, 2H), 3.76 (s, 3H), 3.70-3.57 (bs, 6H), 3.40-3.33 (bs, 2H), 2.93-2.82 (m, 1H), 2.75-2.67 (m, 2H), 2.62-2.53 (m, 2H) ppm.

### Example 7: Preparation of morpholino((1s,3s)-3-(trifluoromethoxy)cyclobutyl)methanone (VIa-1b)

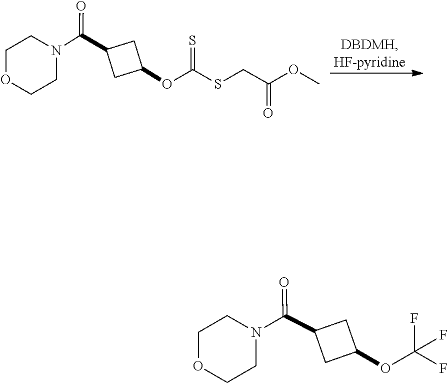 [SMILES: COC(=O)CSC(=S)O[C@H]1C[C@@H](C(=O)N2CCOCC2)C1 ; O=C([C@H]1C[C@@H](OC(F)(F)F)C1)N1CCOCC1]

To a solution of DBDMH (3.5 kg, 12.3 mol, 3.0 equiv) in dichloromethane (6.9 L, 5.0 rel. volumes) stirred at −78° C. in a Teflon flask was added a solution of HF-pyridine (70%, 5.9 kg, 205 mol, 50.0 equiv). The mixture was stirred at this temperature for 1 h before a solution of methyl 2-[3-(morpholine-4-carbonyl)cyclobutoxy]carbothioylsulfanylacetate (1370 g, 4.1 mol, 1.0 equiv) in dichloromethane (5.5 L, 4.0 rel. volumes) was added dropwise at −78° C. The reaction mixture was stirred at this temperature for 30 min before the reaction mass was allowed to warm to 20-30° C., whereupon the mixture was stirred at this temperature for 2 h at which point HPLC showed no remaining starting material. (When complete the reaction was held overnight at this point). Subsequently, the reaction mixture was quenched by slow addition onto ice cold water (13.7 L, 10 volumes) and sodium thiosulphate solution (10%, 9.6 L, 7.0 volumes). The resulting layers were separated, and aqueous layer was extracted with dichloromethane (2×2.7 L, 2×2 volumes). The combined organic layers were neutralized to approx. pH 10 with a solution of saturated sodium bicarbonate (4.1 L, approx. 3.0 volumes). Then washed with a solution of hydrochloric acid (1.5N, 3.4 L, 2.5 volumes) and water (2×3.4 L, 2×2.5 volumes) before being concentrated in vacuo. The resulting crude (1255 g, 1.0 equiv) was then charged with 10% MTBE in heptane (3.8 L, 3.0 rel. volumes) and cooled to 0-5° C. The mixture was then stirred for 1 h before being filtered. The solid on filter was washed with pre-cooled 10% MTBE in heptane (1.3 L, 1.0 volume), and then the resulting solid was dried in vacuo at 40° C. Yield: 975 g (94%)

### Example 8: Preparation of (1s,3s)-3-(trifluoromethoxy)cyclobutane-1-carboxylic acid tert-butylamine Complex (VII-1a)

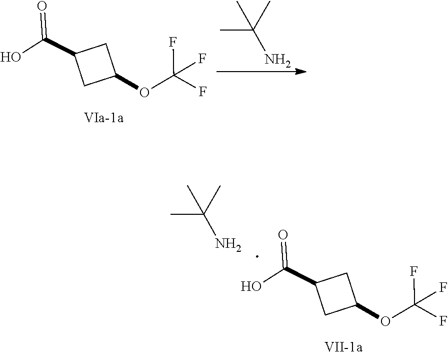

EtOAc (55.6 kg) and VIa-1a (55.6 kg, 292 mol, 1.0 eq) were combined in a drum and then charged to a reactor via filter. EtOAc (222.4 kg) was then charged to the vessel via filter, and the contents were mixed for about 0.5 h. At about 15˜35° C., tert-butylamine (24.5 kg, 335 mol, 1.15 eq) was charged to the reactor via pump, and stirred at about 15˜35° C. for about 2 h. The reaction mixture was warmed to about 75˜78° C., agitated for about 1 h, and then cooled to about 20˜25° C. VII-1a was isolated by centrifugation and washed with EtOAc (55.6 kg). The product was dried at not more than about 55° C. to give solid VII-1a. Yield: 70.5 kg (93.5% yield).

¹H NMR (DMSO-d₆, 400 MHz) δ=6.05 (s, 1H), 4.57-4.53 (m, 1H), 2.42-2.25 (m, 3H), 2.20-2.11 (m, 2H), 1.21 (s, 9H) ppm. ¹⁹F NMR (DMSO-d₆, 376 MHz) δ=−57.8 ppm.

### Example 9: Preparation of tert-butyl (3-(2-((1s,3s)-3-(trifluoromethoxy)cyclobutane-1-carbonyl)hydrazine-1-carbonyl)bicyclo[1.1.1]pentan-1-yl)carbamate monohydrate (IX-1a)

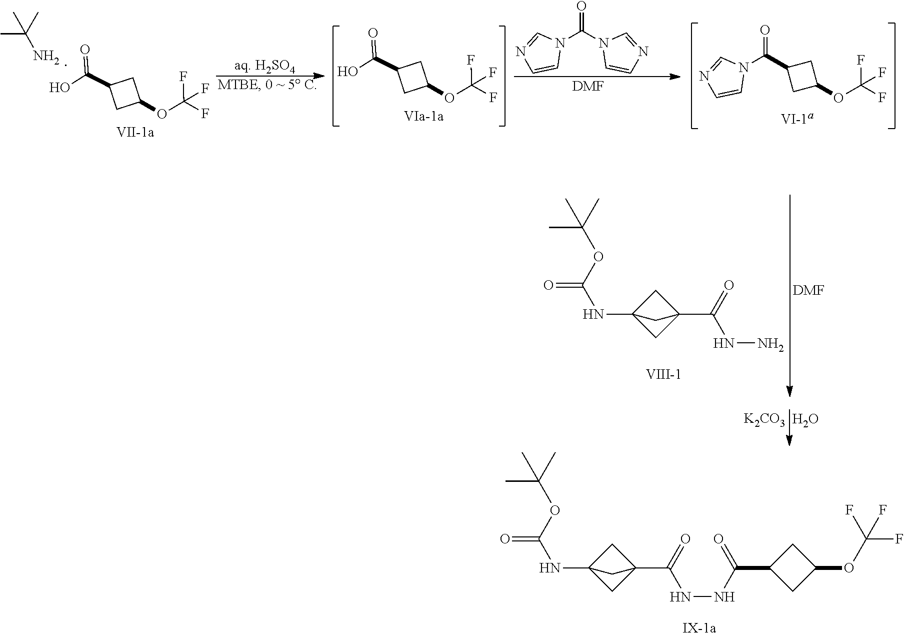

Purified water was charged to a reactor and cooled to about 0˜5° C. Sulfuric acid was slowly charged while maintaining an internal temperature of not more than about 35° C., and then cooled to about 0˜5° C. VII-1a and MTBE were charged and then adjusted at about 20˜25° C. The contents were agitated at about 20˜25° C. for not less than about 1.5 hours to provide a solution containing in-situ-generated VIa-1a. The layers were settled and then separated. The organic layer was concentrated under vacuum until no distillate was observed while maintaining a jacket temperature of not more than about 50° C. (water content was not more than 0.5 w/w %).

1,1′-Carbonyldiimidazole (CDI) and N,N-dimethylformamide (DMF) were charged to another reactor and cooled to about 0˜5′ C. The solution containing in-situ generated VIa-1a was transferred to the CDI slurry for not less than about 0.5 hour, and then rinsed with DMF. The contents were agitated at about 0˜5° C. for not less than about 2 hours to form activated VI-1^{a}. Once the reaction was completed, VIII-1 was charged to the VI-1a then rinsed with DMF at about 0˜5′ C. The contents were agitated at about 0˜5° C. then adjusted to about 20˜25° C. for not less than about 0.5 hour. The contents were agitated at about 20˜25° C. for not less than about 3 hours until the reaction was completed. Potassium carbonate (K₂CO₃) and purified water were charged to another reactor and heated to about 40˜45° C. Once the reaction was completed, the reaction mixture was slowly transferred to the aq. K₂CO₃ for not less than about 1 h while maintaining an internal temperature of about 40˜45° C.. The contents were adjusted to about 20˜25° C. then agitated at about 20˜25° C. for not less than about 1 hour. The slurry was filtered then washed with purified water twice. The wet cake was dried at not more than about 80° C. (target 50° C.) under vacuum until the water content was not more than about 5.0 w/w %. ¹H NMR (d₆-DMSO, 600 MHz): 9.74 (s, 2H), 7.58-7.32 (m, 1H), 4.79 (p, 1H, J=7.5 Hz), 2.68 (p, 1H, J=7.5 Hz), 2.53-2.49 (m, 2H), 2.29-2.24 (m, 2H), 2.12-2.00 (m, 6H), 1.28 (s, 9H) ppm.

### Optional Purification

IX-1a dry cake and purified water were charged to a reactor. The contents were agitated at about 20˜25° C. for not less than about 6 hours. The slurry was filtered then washed with purified water twice. The wet cake was dried at not more than about 80° C. (target 50° C.) under vacuum until the water content was not more than 5.0 w/w %. If the purity of dry cake was less than 96.0 a/a % by HPLC, the optional purification was repeated.

### Example 10: Preparation of tert-butyl (3-(5-((1s,3s)-3-(trifluoromethoxy)cyclobutyl)-1,3,4-oxadiazol-2-yl)bicyclo[1.1.1]pentan-1-yl)carbamate (X-1a)

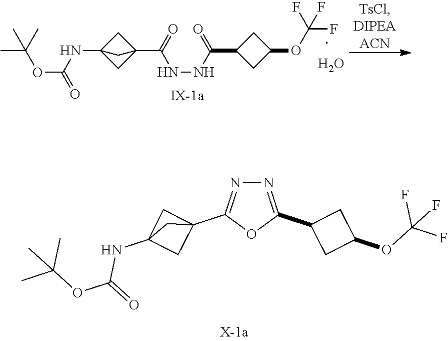

IX-1a, 4-toluenesulfonyl chloride (TsCl), and acetonitrile (ACN) were charged to a reactor. The contents were agitated and adjusted to about 20˜25° C. N,N-Diisopropylethylamine (DIPEA) was added and rinsed with ACN. The contents were adjusted to about 20˜25° C. and agitated at about 20˜25° C. for not less than about 12 hours. Once the reaction was completed, potassium hydroxide (KOH) solution (aq. 6%) was added to the reaction mixture while maintaining an internal temperature of not more than about 30° C. (target 20˜25° C.). The resulting slurry was adjusted to about 20˜25° C. then agitated at about 20˜25° C. for not less than about 2 hours until TsC1 content was not more than about 10 ppm. The slurry was filtered then washed with aq. ACN and purified water. The wet cake was dried at not more than about 60° C. (target 50° C.) under vacuum until the water content was not more than about 0.2 w/w % by Karl Fischer method. ¹H NMR (CDCl₃, 600 MHz): 5.12 (s, 1H), 4.68 (p, 1H, J=8 Hz), 3.30 (p, 1H, J=9 Hz), 2.86-2.82 (m, 2H), 2.68-1.63 (m, 2H), 2.58 (s, 6H), 1.45 (s, 9H) ppm.

### Example 11: Preparation of 3-(5-((1s,3s)-3-(trifluoromethoxy)cyclobutyl)-1,3,4-oxadiazol-2-yl)bicyclo[1.1.1]pentan-1-amine (XI-1a)

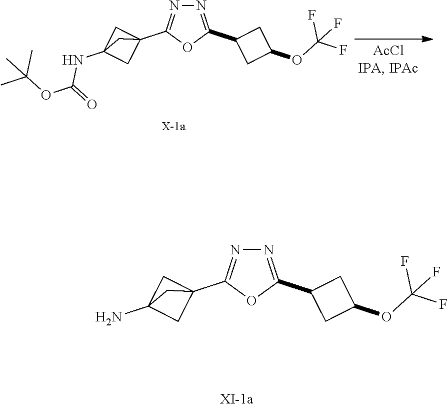

Acetyl chloride (AcCl) and isopropyl acetate (IPAc) were charged to a reactor and cooled to about 0˜5° C. An IPAc solution of isopropyl alcohol (IPA) was charged into the AcCl solution for not less than about 1 h while maintaining an internal temperature of not more than about 20° C. under nitrogen (N₂) atmosphere. The contents were adjusted to about 20˜25° C., and then agitated at about 20˜25° C. for not less than about 2 h, to provide an in-situ generated hydrogen chloride (HCl) solution. X-1a and IPAc were charged to another reactor and cooled to about 0˜5° C. The HCl solution was added to the X-1a/IPAc slurry while maintaining an internal temperature of not more than about 15° C. under N₂ atmosphere, and then rinsed with IPAc. The contents were adjusted to about 40˜45° C. then agitated at about 40˜45° C. for not less than about 2 h. Once the reaction was completed, the reaction mixture was adjusted to about 20˜25° C. and slowly added to a pre-cooled (0˜5° C.) aq. potassium phosphate (K₃PO₄) solution while maintaining an internal temperature of not more than about 20° C., and then rinsed with IPAc twice. The mixture was adjusted to about 20˜25° C. and agitated for not less than about 1 h. The layers were settled and separated. The aqueous layer was extracted with IPAc. If the 3-amino-N′-(cis-3-(trifluoromethoxy)cyclobutane-1-carbonyl)bicyclo[1.1.1]pentane-1-carbohydrazide content was more than 1.0%, the combined organic layer was additionally washed with aq. KOH. The combined organic layer was concentrated to a target volume to 3 V at not more than about 50° C. under vacuum. The resulting concentrate was diluted with IPAc. The contents were adjusted to about 20-25° C. then filtered through 1 μm of disk filter and 1 μm of cartridge filter then rinsed with IPAc. The filtrate was concentrated to a target volume of 1.6 V while maintaining a jacket temperature of not more than about 50° C. The content was heated to about 40˜50° C. then agitated for not less than about 1 hour. N-heptane was slowly added to the content, and then cooled to about 20˜25° C. for not less than about 1 hour. N-heptane was slowly added for not less than about 1 hour then the slurry was agitated at about 20˜25° C. for not less than about 2 hours. The slurry was cooled to about 0˜5° C. then agitated for not less than about 3 hours at about 0˜5° C. The slurry was filtered then washed with pre-cooled mixture of n-heptane/IPAc (16:1, v/v). The wet cake was dried at not more than about 50° C. (target 25° C.) under vacuum until the water content was not more than 0.6 w/w % by Karl Fischer method and the sum of residual solvents (chloroethane, 2-chloropropane, tert-butylamine, IPAc, n-heptane, DIPEA) was not more than 5,000 ppm. ¹H NMR (CDCl₃, 600 MHz): 4.69 (quint, 1H, J=8 Hz), 3.29 (quint, 1H, J=9 Hz), 2.85-2.80 (m, 2H), 2.68-2.63 (m, 2H), 2.28 (s, 6H) ppm. The DSC of the free-base is shown in FIG. 12.

### Example 12: Preparation of 4-chlorophenoxyacetic acid salt of 3-(5-((1s,3s)-3-(trifluoromethoxy)cyclobutyl)-1,3,4-oxadiazol-2-yl)bicyclo[1.1.1]pentan-1-amine

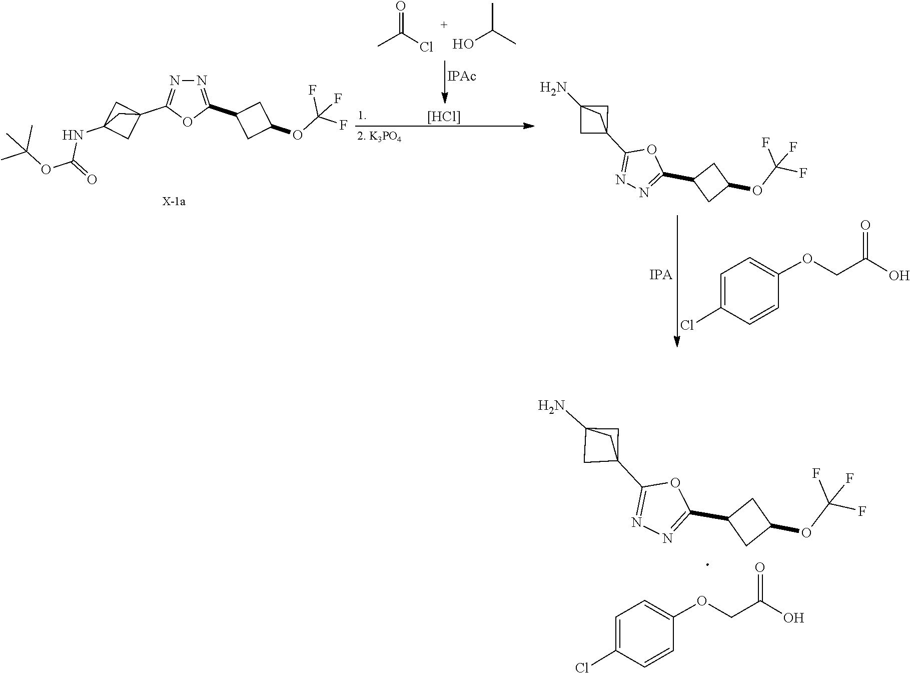

Acetyl chloride (AcCl) and isopropyl acetate (IPAc) were charged to a reactor and cooled to about 0˜5° C. An IPAc solution of isopropyl alcohol (IPA) was charged into the AcCl solution for not less than about 1 h while maintaining an internal temperature of not more than about 20° C. under nitrogen (N₂) atmosphere. The contents were adjusted to about 20˜25° C., and then agitated at about 20˜25° C. for not less than about 2 h, to provide an in-situ generated hydrogen chloride (HCl) solution. X-1a and IPAc were charged to another reactor and cooled to about 0˜5° C. The HCl solution was added to the X-1a/IPAc slurry while maintaining an internal temperature of not more than about 15° C. under N₂ atmosphere, and then rinsed with IPAc. The contents were adjusted to about 40˜45° C. then agitated at about 40˜45° C. for not less than about 2 h. Once the reaction was completed, the reaction mixture was adjusted to about 20˜25° C. and slowly added to a pre-cooled (0˜5° C.) aq. potassium phosphate (K₃PO₄) solution while maintaining an internal temperature of not more than about 20° C., and then rinsed with IPAc twice. The mixture was adjusted to about 20˜25° C. and agitated for not less than about 1 h. The layers were settled and separated. The aqueous layer was extracted with IPAc. The combined organic layer was concentrated to a target volume to 3 V at not more than about 50° C. under vacuum until the water content was not more than 0.3% w/w. The resulting concentrate was diluted with IPAc. The contents were adjusted to about 20˜25° C. then filtered through 1 m of disk filter and 1 m of cartridge filter then rinsed with IPAc. The filtrate was concentrated to a target volume of 2.5 V while maintaining a jacket temperature of not more than about 50° C. The content was adjusted to about 20-25° C. 4-chlorophenoxyacetic acid solution in IPA was separately prepared, and was then added slowly to the concentrated XI-1a/IPAc mixture over not less than 2 hours at 20-25° C. and rinsed with IPA. The contents were then adjusted to 0-5° C. over 1 hour and mixed for not less than 1 hour. The slurry was filtered under vacuum and deliquored. The vessel was rinsed with pre-cooled IPA, and the cake was dried at not more than 50° C. under vacuum. ¹H NMR (600 MHz, d₆-DMSO)=7.32-7.30 (m, 2H), 6.93-6.91 (m, 2H), 4.88 (quint, 1H, J=7.2 Hz), 4.61 (s, 2H), 3.39 (quint, 1H, J=6.0 Hz), 2.85-2.82 (m, 2H), 2.50-2.45 (m, 2H), 2.12 (s, 6H).

Referring to FIG. 9, the DSC of the 4-chlorophenoxyacetic acid salt of 3-(5-((1s,3s)-3-(trifluoromethoxy)cyclobutyl)-1,3,4-oxadiazol-2-yl)bicyclo[1.1.1]pentan-1-amine comprises an endothermic peak having a onset temperature of about 162° C.

### Example 13: Preparation of dioxalate salt of 3-(5-((1s,3s)-3-(trifluoromethoxy)cyclobutyl)-1,3,4-oxadiazol-2-yl)bicyclo[1.1.1]pentan-1-amine

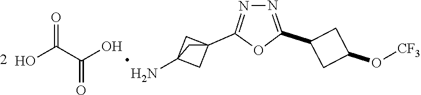

To a 3-neck 100 mL round bottom flask, 5 g of Compound XI-1a, 11.56 g (10 eq.) of oxalic acid, and 50 mL (10 V) of MeCN were added. The contents were heated to 40-45° C. and agitated for 19 hours. The contents were cooled to 20-25° C. and agitated for about 1 hour. The contents were filtered and rinsed with 20 mL (4 V) of MeCN. The wet cake was dried under a vacuum in a 30° C. oven to yield the title compound (97% yield). ¹H NMR (d₆-DMSO, 600 MHz): 4.89 (quint, 1H, J=7.8 Hz), 3.41 (quint, 1H, J=8.4 Hz), 2.85-2.81 (m, 2H), 2.51-2.46 (m, 2H), 2.43 (s, 6H) ppm. The DSC of the salt is shown in FIG. 14.

### Example 14: Preparation of 2-(4-chlorophenoxy)-N-(3-(5-((1s,3s)-3-(trifluoromethoxy)cyclobutyl)-1,3,4-oxadiazol-2-yl)bicyclo[1.1.1]pentan-1-yl)acetamide (I)

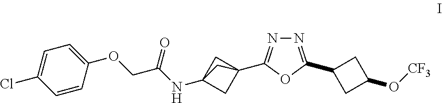

XI-1a, 2-(4-chlorophenoxy)acetic acid (XII-1a), and 2-MeTHF were charged to a reactor under N₂ condition and cooled to 0˜5° C. TEA was added while maintaining an internal temperature of not more than about 10° C. under N₂ condition and rinsed with 2-methyltetrahydrafuran (2-MeTHF). The contents were agitated at about 0˜5° C. for not less than about 20 minutes. Diphenylphosphinic chloride in 2-MeTHF solution is added slowly while maintaining an internal temperature of not more than about 10° C. under N₂ condition and rinsed with 2-MeTHF. The contents were warmed to about 20˜25° C. and then agitated for not less than about 1 hour until the reaction was completed. The contents were cooled to about 0˜5° C. and then aqueous 10% K₂CO₃ was added while maintaining an internal temperature of not more than about 10° C. After phase separation, the organic layer was successively washed with aqueous 10% K₂CO₃ and 5% K₂CO₃. The organic layer was concentrated to a target volume 3 V. 2-MeTHF was added and then the contents were concentrated to a target volume 3 V to control the water content to not more than about 0.3 w/w %. IPA was added and then the contents were heated to about 60˜70° C. to dissolve all solids. The contents were filtered at about 60˜70° C. through cartridge filter and rinsed with pre-heated IPA (60˜70° C.). The filtrate was concentrated to a target volume 4 V. IPA was added and concentrated to a target volume 4 V to control the residual 2-MeTHF relative to IPA to not more than 1% by GC. The contents were adjusted to about 20˜25° C. n-Heptane was added and then heated to about 60˜80° C. to dissolve all solids. The contents were adjusted to about 62˜70° C. Seed crystal was charged and agitated for not less than about 0.5 hour. n-Heptane was added while maintaining an internal temperature of about 60˜65° C. The contents were cooled to about 0-5° C. over 12 hour (5° C. per hour). The slurry was agitated for not less than 2 hours. The slurry was filtered and washed with pre-cooled IPA/n-heptane mixture. The wet cake was dried at 25° C. under vacuum. If any individual impurity except 2-(4-chlorophenoxy)-N-(3-(5-(trans-3-(trifluoromethoxy)cyclobutyl)-1,3,4-oxadiazol-2-yl)bicyclo[1.1.1]pentan-1-yl)acetamide was more than 0.12%, recrystallization was performed.

¹H NMR (600 MHz, MeCN-d₃): 7.65 (s, 1H), 7.31 (d, 2H, J=9 Hz), 6.96 (d, 2H, J=9 Hz), 4.79 (m, 1H), 4.40 (s, 2H), 3.34-3.29 (m, 1H), 2.96-2.82 (q, 2H, J=9 Hz), 2.59-2.54 (q, 2H, J=10 Hz), 2.53 (s, 6H) ppm.

### Example 15: Alternative Preparation of 2-(4-chlorophenoxy)-N-(3-(5-((1s,3s)-3-(trifluoromethoxy)cyclobutyl)-1,3,4-oxadiazol-2-yl)bicyclo[1.1.1]pentan-1-yl)acetamide (I)

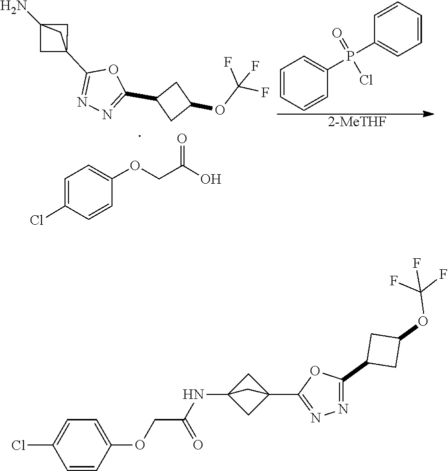

4-chlorophenoxyacetic acid salt of 3-(5-((1s,3s)-3-(trifluoromethoxy)cyclobutyl)-1,3,4-oxadiazol-2-yl)bicyclo[1.1.1]pentan-1-amine (XI-1a) and 2-MeTHF were charged to a reactor under N₂ condition and cooled to 0˜5° C. TEA was added while maintaining an internal temperature of not more than about 10° C. under N₂ condition and rinsed with 2-methyltetrahydrafuran (2-MeTHF). The contents were agitated at about 0˜5° C. for not less than about 20 minutes. Diphenylphosphinic chloride in 2-MeTHF solution is added slowly while maintaining an internal temperature of not more than about 10° C. under N₂ condition and rinsed with 2-MeTHF. The contents were warmed to about 20-25° C. and then agitated for not less than about 1 hour until the reaction was completed. The mixture was cooled to about 0-5° C. and charged with 10% aqueous potassium carbonate solution over not less than 1 h at not more than 10° C. The temperature was increased to about 45-50° C. and agitated for not less than 1 hour. The layers were separated, and the organic phase was charged again with 10% aqueous potassium carbonate and agitated at about 45-50° C. for not less than 0.5 hours. The layers were separated, and the organic phase was charged with 5% aqueous potassium carbonate solution and agitated at about 45-50° C. for not less than 0.5 hours. The layers were separated, and the organic layer was concentrated to about 3.6V under vacuum at not more than 50° C. MeTHF was charged, and the mixture was concentrated to about 3.6V under vacuum at not more than 50° C. until the water content was not more than 1% w/w. MeTHF was charged to the vessel and the mixture was cooled to 20-25° C. The solution was filtered through a cartridge filter, and the filtrate was concentrated to about 2.4V under vacuum at not more than 50° C. The solvent was exchanged with IPA until the MeTHF:IPA ratio was not more than 20 wt %. The mixture temperature was adjusted to about 45-50° C. Heptane was charged, and the contents were adjusted to 60-70° C. to generate a homogeneous solution. The temperature was then adjusted to 50-55° C. and agitated for not less than 2 hours. Additional heptane was charged, and the temperature was then adjusted to about 0-5° C. and agitated for not less than 2 hours. The solids were isolated by filtration and washed with precooled IPA/heptane solution, and thoroughly deliquored. The solids were dried under vacuum at not more than 25° C.

### Example 16: Salt Screening for the Compound of Formula VIa-1a

Selected solvents (ethyl acetate (EtOAc) or toluene) were added into vials containing about 22 mg of the compound of Formula VIa-1a and an equal molar amount of selected bases to prepare slurries, as illustrated in Table 2. The slurries were stirred at room temperature (RT) for 2 to 3 days. If solids precipitated out, the solids were retrieved by centrifugation and then vacuum dried at RT for XRPD analysis. Samples with no precipitation after solution-mediated reaction were cooled at −15° C. in fridge for 1 day. If solids precipitated out at that time, the solids were retrieved by centrifugation and then vacuum dried at RT for XRPD analysis. If no solids obtained after cooling, n-heptane was added and the mixture further stirred for 2 days. Solutions with no precipitation after n-heptane addition were then uncapped for slow evaporation under ambient until solids formed. The results were summarized in Table 2 below:

TABLE 2

| | Solvent | |
|---|---|---|
| Base | Ethyl Acetate | Toluene |
| t-Butyl amine (TBA) | TBA salt Form A | TBA salt Form A |
| Magnesium(II) Acetate | Mg salt Form A + Mg(OAc)₂ | Magnesium(II) Acetate tetrahydrate |
| tetrahydrate | | |
| L-Lysine | L-Lysine salt Form A | yellow gel |
| Piperazine | Piperazine salt Form A | Piperazine salt Form A |
| Sodium hydroxide | Na salt Form A | Na salt Form A |
| Calcium(II) Acetate | Ca salt Form B | Ca salt Form A |
| Dicyclohexylamine (DHCA) | DCHA salt Form A | gel |
| Zinc(II) Acetate | Zn salt Form A | Zn salt Form B |
| L-Arginine | L-Arginine salt Form A | L-Arginine salt Form A |
| N,N,N′,N′- | oil | oil |
| Tetramethylethylenediamine | | |
| Triisobutylamine | gel | gel |
| 4-Methylmorpholine | oil | oil |
| Dibutylamine | oil | oil |
| TrimethamineTromethamine | TMA salt Form A | TMA salt Form A |
| (TMA) | | |
| Lithium (Li) hydroxide | Li salt Form A | Li salt Form B |
| monohydrate | | |
| Dibenzylamine (DBA) | DBA salt/cocrystal Form B | DBA salt/cocrystal Form A |
| Dehydroabietylamine (DAA) | DAA salt Form A | DAA salt Form A |
| Ethanolamine | Ethanolamine salt Form A | Ethanolamine salt Form A |

### Example 17: Scale Up for the Certain Salts of the Compound of Formula VIa-1a

### Compound VIa-1a, DCHA Salt Form A

Around 2.5 mmol of the compound of Formula VIa-1a and DCHA (1.1 eq) were added into 20 mL EtOAc and stirred at RT for 2 days. Solids were retrieved by centrifugation and vacuum dried at 40° C. for about 1 day. The solids were identified as Compound of Formula VIa-1a, DCHA salt Form A.

### Compound VIa-1a, L-Arginine Salt Form A

Around 2.5 mmol of the compound of Formula VIa-1a and L-Arginine (1.1 eq) were added into 20 mL EtOAc, and stirred at RT for about 5 days. After stirring at RT for 2 days, the wet cake was characterized by XRPD and identified as Compound of Formula VIa-1a, L-Arginine salt Form B. After stirring at RT for another about 3 days, the wet cake was characterized by XRPD and identified as Compound of Formula VIa-1a, L-Arginine salt Form A. Solids were retrieved by centrifugation and vacuum dried at 40° C. for 2 days. Then obtained solids were further vacuum dried at 60° C. for 2 days. The solids were identified as Compound of Formula VIa-1a, L-Arginine salt Form A.

### Compound VIa-1a, TMA Salt Form A

Around 2.5 mmol of the compound of Formula VIa-1a and tromethamine (TMA) (1.1 eq) were added into 20 mL EtOAc and stirred at RT for about 2 days. Solids were retrieved by centrifugation and vacuum dried at 40° C. for about 1 day. The solids were identified as Compound of Formula VIa-1a, TMA salt Form A. However, the XRPD pattern comprises an extra peak at 18.1° as compared to Compound VIa-1a, TMA salt Form A prepared from that of Example 15.

### Example 18: Characterization of Salt Forms

As illustrated in Table 2, eighteen salts were obtained from the above experiment. These salts were characterized by XRPD, TGA, and DSC. Data for select salts were presented above. Additional results are summarized in Table 3.

TABLE 3

| | TGA weight loss/ | DSC peak | |
|---|---|---|---|
| Salt form | Temperature range (° C.) | temperature (° C.) | Phase |
| TBA salt Form A | 0.0/RT-70 | Endo: 170.6 | Anhydrate |
| Mg salt Form A | 20.6/RT-170 | Endo: 76.0 | Solvate/Hydrate |
| L-Lysine salt Form A | 4.8/RT-150 | Endo: 92.0, 181.8 | Hydrate |
| Piperazine salt Form A | 8.5/RT-110 | Endo: 123.7, 139.9 | Hydrate |
| Ca salt Form A | 5.6/RT-150 | Endo: 135.2, 163.0; | Solvate/Hydrate |
| | | Exo: 195.3 | |
| Ca salt Form B | 6.1/RT-210 | Endo: 241.1; | Solvate/Hydrate |
| | | Exo: 107.3 | |
| DCHA salt Form A | 0.8/RT-100 | Endo: 138.3 | Anhydrate |
| Zn salt Form A | 0.7/RT-150 | Endo: 192.6, 199.1 | Anhydrate |
| Zn salt Form B | 5.4/RT-150 | Endo: 103.5, 183.2 | Hydrate |
| L-Arginine salt Form A | 1.0/RT-150 | Endo: 164.1, 166.6 | Anhydrate |
| Na salt Form A | 9.2/RT-140 | Endo: 64.6, 137.4 | Hydrate |
| TMA salt Form A | 0.0/RT-100 | Endo: 136.3 | Anhydrate |
| Li salt Form A | 11.6/RT-150 | Endo: 86.1, 215.5 | Solvate |
| Li salt Form B | 1.0/RT-150 | Endo: 127.0, 209.2 | Anhydrate |
| Ethanolamine salt Form A | 1.0/RT-100 | Endo: 69.3 | Anhydrate |
| DAA salt Form A | 0.0/RT-100 | Endo: 152 | Anhydrate |
| Dibenzylamine (DBA) | 0.7/RT-90 | Endo: 88.5, 94.5 | Anhydrate |
| salt/cocrystal Form A | | | |
| DBA salt/cocrystal Form B | 0.7/RT-90 | Endo: 95.1 | Anhydrate |

Several salts were further investigated for hygroscopicity, purity, and solid form stability. To determine solid form stability, glass weighing bottles containing about 20 mg samples were kept under (1) 60° C., (2) 25° C./60% RH, and (3) 40° C./75% RH conditions, respectively. After 1 week and 2 weeks, respectively, XRPD and GC analyses were conducted to investigate the physical and chemical stability of the selected forms under corresponding conditions. The results were summarized in Table 4 below.

TABLE 4

| | | Compound | Compound | Compound | Compound |
|---|---|---|---|---|---|
| | | VIa-1a, TBA | VIa-1a, DCHA | VIa-1a, L-Arginine | VIa-1a, TMA |
| Form | | salt Form A | salt Form A | salt Form A | salt Form A^{#} |
| Identification | | Anhydrate | Anhydrate | Anhydrate | Anhydrate |
| TGA weight loss %/ | | 0.0/RT-70 | 0.9/RT-100 | 0.3/RT-130 | 0.0/RT-100 |
| Temperature range (° C.) | | | | | |
| DSC peak temperature | | Endo: 170.6 | Endo: 138.7 | Endo: 161.4 | Endo: 117.7, |
| (° C.) | | | | | 134.2/136.1 |
| Hygroscopicity | | non- | non- | hygroscopic | non- |
| | | hygroscopic | hygroscopic | | hygroscopic* |
| Purity (%) | | 99.8 | 99.9 | 99.1 | 99.4 |
| 60° C. 2 weeks | XRPD | Sublimed^{&} | Sublimed | additional peak | No change |
| stability | | | | at 18.1° | |
| | Purity (%) | NA | NA | 99.2 | 99.3 |
| 25° C./60% RH | XRPD | NA | No change | No change | No change |
| 2-weeks stability | Purity (%) | NA | 99.7 | 98.8 | 99.4 |
| 40° C./75% RH | XRPD | NA | No change | Deliquesced | No change |
| 2-weeks stability | Purity (%) | NA | 99.3 | NA | 99.3 |

^{#}Mixture containing TMA salt Form A and an unidentified Form based on DSC results
^{&}Completely sublimed by stressed at 60° C. for 1 day

### Compound VIa-1a TBA Salt Form A

Compound VIa-1a TBA Salt Form A was obtained by solution-mediated reaction with an equal molar quantity of t-butylamine in ethylacetate (EtOAc) or toluene. XRPD pattern of Compound VIa-1a TBA Salt Form A was described above with respect to FIG. 1. As shown in FIG. 2, no weight loss was observed up to 70° C. in TGA curve. The sample may sublime above 100° C. DSC profile showed an endothermic peak at around 171° C. DSC analysis was conducted using Tzero hermetic pan without a pinhole. ¹H NMR indicated salt formation between the compound of Formula VIa-1a and t-butylamine at a ratio of about 1:1, without organic solvent. Form A is an anhydrate.

### Compound Via-1a DCHA Salt Form A

Compound VIa-1a DCHA salt Form A was obtained by solution-mediated reaction with equal molar quantity of dicyclohexylamine (DCHA) in EtOAc. XRPD pattern of Compound VIa-1a DCHA salt Form A was described above with respect to FIG. 3. As shown in FIG. 4, 0.8% weight loss was observed up to 100° C. in TGA curve, and the sample might sublime at above 110° C. DSC analysis was conducted by using Tzero hermetic pan without a pinhole. DSC curve showed an endothermic peak at around 139° C. corresponding to melting. ¹H NMR result suggested salt formation between the compound of Formula VIa-1a and DCHA at a ratio of about 1:1, with no organic solvent. Compound VIa-1a DCHA salt Form A is an anhydrate. DVS analysis suggests Compound VIa-1a DCHA salt Form A to be non-hygroscopic; and no form conversion was detected after DVS test. The PLM image showed that Compound VIa-1a DCHA salt Form A crystals were needle-like. Form B is an anhydrate.

### Compound VIa-1a, L-arginine Salt Form A

Compound VIa-1a, L-arginine salt Form A was obtained by solution-mediated reaction with an equal molar quantity of L-Arginine in EtOAc or toluene. XRPD pattern of Compound VIa-1a, L-arginine salt Form A was described above with respect to FIG. 5. As shown in FIG. 6, 1.0% weight loss was observed up to 150° C. in TGA curve, and the sample might decompose by heating above 170° C. DSC curve showed an endothermic peak at around 161° C. corresponding to melting. ¹H NMR result indicated salt formation between the compound of Formula VIa-1a and L-Arginine at a ratio of about 1:1 with no organic solvent. Compound VIa-1a, L-arginine salt Form A is likely to be an anhydrate. DVS analysis of Compound VIa-1a, L-arginine salt Form A showed about 28% weight gain at 80% RH and about 74% weight gained at 95% RH, indicating high hygroscopicity. Compound VIa-1a, L-arginine salt Form A deliquesced with high humidity. The PLM image showed that Compound VIa-1a, L-arginine salt Form A crystals were small particles clustered together.

### Compound VIa-1a, TMA Salt Form A

Compound VIa-1a, tromethamine (TMA) salt Form A was obtained by solution-mediated reaction with an equal molar quantity of TMA in EtOAc or toluene. XRPD pattern of Compound VIa-1a, TMA salt Form A was described above with respect to FIG. 7. As shown in FIG. 8, no weight loss was observed up to 100° C. in TGA curve, and the sample might decompose above 135° C. DSC analysis was conducted by using Tzero hermetic pan without a pinhole. DSC curve showed endothermic peaks at around 123° C. and 136° C. ¹H NMR result indicated salt formation between the compound of Formula VIa-1a and TMA at a ratio of about 1:1 with no organic solvent. Compound VIa-1a, TMA salt Form A is likely to be an anhydrate. DVS analysis of Compound VIa-1a, TMA salt Form A showed about 0.1% weight gain at 80% RH, about 47% weight gain at 95% RH, and about 7% weight gain at 0% RH. Compound VIa-1a, TMA salt Form A was non-hygroscopic but may deliquesce with high humidity (>90% RH). PLM image of Compound VIa-1a, TMA salt Form A showed plate-like crystals.

### Example 19: Crystal Structure Determination of 4-Chlorophenoxyacetic Acid Salt of Compound of Formula XI-1a by microED

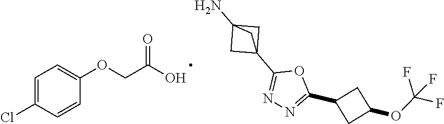

MicroED data was collected using a Thermo Scientific Glacios 200 kV cryo-TEM with a Thermo Scientific Ceta-D detector at 200 kV, 98(2) K, and high vacuum of 10⁻⁷ Pa. The sample was prepared by adding 4-chlorophenoxyacetic acid salt of compound of Formula XI-1a powder on the electron microscope grid, and rapidly freezing the grid in liquid ethane using a Thermo Scientific Vitrobot cryo-EM sample preparation system. Data collection was performed using Thermo Scientific EPU-D. Data was processed with XDS, SHELXT, and SHELXL. Twenty-six sets of diffraction frames were collected from 30 different particles. They were indexed and integrated by XDS. The cell parameters are a=28.94(8) Å, b=12.34(4) Å, c=13.37(3) Å, α=90°, β=108.3(2°), γ=90°, with a volume of V=4532(23) Å 3. Twenty-two of the data sets yield the best quality and were merged by XSCALE. The merged data set had 47570 total reflections, and 3003 of them were independent. The resolution of the reflections was truncated to 0.90 Å. The Rint value was 0.3625. The crystal system was determined to be monoclinic, with a space group of C 2/c (No. 15) and a Z′ of 1.

### Example 20: Single Crystal Structure x-Ray Determination of Compound of Formula VII-1a

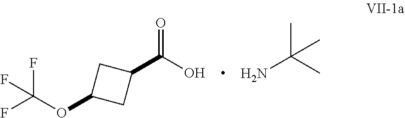

Crystals of compound of Formula VII-1a were obtained by liquid vaporization from an EtOAc solution at room temperature. A suitable single crystal having a size of 0.35×0.08×0.06 mm was mounted on a glass fiber for SCXRD measurement. SCXRD was collected using a SuperNova (Rigaku JPN). The X-ray source is a Cu tube (λ=1.54184 Å). The crystal was kept at a steady temperature at 150.0 K during data collection. Preliminary examination and data collection were performed and analyzed with the CrysAlisPro software package. Cell parameters and an orientation matrix for data collection were retrieved and refined by CrysAlisPro using the setting angles of 1547 reflections in the range 3.235°<θ<74.111°. The final data completeness was 99.7% (θ=66.97°). The structure was solved in the space group P-1 with the XT (Sheldrick, 2015) structure solution program using the intrinsic phasing method and by using Olex2 as the graphical interface. The model was refined with version of XL (Sheldrick, 2015) using full matrix least squares on F² minimization. All non hydrogen atoms were refined anisotropically. The positions of all hydrogen atoms were calculated geometrically and refined using the riding model. The crystal structure representations and the thermal ellipsoids drawings were generated by Diamond. The crystal system of the VII-1a single crystal is triclinic crystal system and P-1 space group. The cell parameters are: a=6.4961(5) Å, b=7.4117(5) Å, c=13.9567(11) Å, α=101.253(6) °, β=91.442(6) °, γ=100.968(6) °, V=645.66(9) Å³.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed:

1. A salt of a compound of Formula VIa-1a: 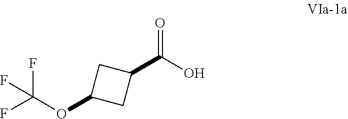
wherein the salt comprises an amine selected from the group consisting of t-butylamine, L-lysine, arginine, piperazine, dicyclohexylamine, tromethamine, ethanolamine, diethanolamine, N,N,N′,N′-tetramethylethylenediamine, triisobutylamine, 4-methylmorpholine, dibutylamine, tromethamine, dehydroabietylamine, N-methyldicyclohexylamine, diethylamine, diisopropylethylamine, diisopropylamine, imidazole, 1,4-diazabicyclo[2.2.2]octane (DABCO), ammonia, and dibenzylamine, or a cation selected from magnesium, sodium, potassium, calcium, zinc, lithium, cesium, tetramethylammonium, and ammonium.

2. The salt of claim 1, having a Formula of VII-2a: 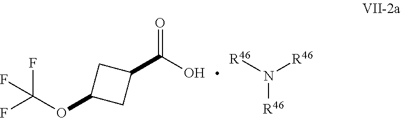
wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

3. The salt of claim 1, having a Formula VII-1a:

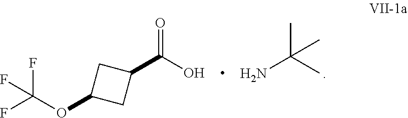

4. The salt of claim 1, having a Formula VII-1b:

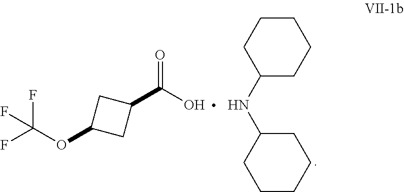 [SMILES: O=C(O)[C@H]1C[C@@H](OC(F)(F)F)C1 ; C1CCC(NC2CCCCC2)CC1]

5. The salt of claim 1, having a Formula VII-1c:

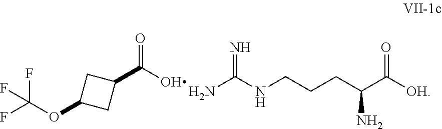 [SMILES: O=C(O)[C@H]1C[C@@H](OC(F)(F)F)C1 ; N=C(N)NCCC[C@H](N)C(=O)O]

6. The salt of claim 1, having a Formula VII-1d:

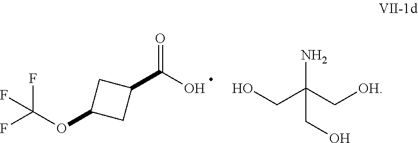 [SMILES: O=C(O)[C@H]1C[C@@H](OC(F)(F)F)C1 ; NC(CO)(CO)CO]

7. Form A polymorph of a t-butylamine (TBA) salt of (1s,3s)-3-(trifluoromethoxy)cyclobutane-1-carboxylic acid (Compound VIa-1a, TBA salt Form A), that exhibits an X-ray powder diffraction pattern having one or more peaks selected from 6.6, 11.6, 12.1, 15.6, 19.8, 20.8, 26.5, and 27.3° 2q±0.2° 2q, wherein the X-ray powder diffraction pattern is made using Cu-Kα radiation.

8. The Compound VIa-1a, TBA salt Form A polymorph of claim 7, further characterized by:
i) the X-ray powder diffraction pattern further comprising one or more peaks selected from 13.2, 14.4, 15.8, 17.2, 22.0, 23.2, 24.3, 32.9, 33.2, and 36.7° 2q±0.2° 2q;
ii) a diffractogram substantially as shown in FIG. 1;
iii) a differential scanning calorimetry (DSC) comprising an endotherm at about 171° C.; or
iv) a thermogravimetric analysis (TGA) comprising a thermogram substantially as shown in FIG. 2.

9. Form A polymorph of a dicyclohexylamine (DCHA) salt of (1s,3s)-3-(trifluoromethoxy)cyclobutane-1-carboxylic acid (Compound VIa-1a, DCHA salt Form A), that exhibits an X-ray powder diffraction pattern having one or more peaks selected from 6.7, 10.6, 17.2, 19.0, and 19.6° 2q±0.2° 2q, wherein the X-ray powder diffraction pattern is made using Cu-Kα radiation.

10. The Compound VIa-1a, DCHA salt Form A polymorph of claim 9, further characterized by:
i) the X-ray powder diffraction pattern further comprising one or more peaks selected from 13.5, 15.1, 20.3, 21.4, 27.2, 28.0, 28.8, 35.4, 36.8, and 39.0° 2q±0.2° 2q;
ii) a diffractogram substantially as shown in FIG. 3;
iii) a differential scanning calorimetry (DSC) comprising an endotherm at about 139° C.; or
iv) a thermogravimetric analysis (TGA) comprising a thermogram substantially as shown in FIG. 4.

11. Form A polymorph of a tromethamine (TMA) salt of (1s,3s)-3-(trifluoromethoxy)cyclobutane-1-carboxylic acid (Compound VIa-1d, TMA salt Form A), that exhibits an X-ray powder diffraction pattern having one or more peaks selected from 6.6, 13.2, 19.9, 20.1, 21.8, and 26.7° 2q±0.2° 2q, wherein the X-ray powder diffraction pattern is made using Cu-Kα radiation.

12. The Form A polymorph of claim 11, further characterized by:
i) the X-ray powder diffraction pattern further comprising one or more peaks selected from 15.9, 17.8, 18.9, 19.5, 20.9, 22.5, 28.4, 29.8, 33.5, 34.3, and 34.8° 2q±0.2° 2q;
ii) a diffractogram substantially as shown in FIG. 7;
iii) a differential scanning calorimetry (DSC) comprising an endotherm at about 123° C. and about 136° C.; or
iv) a thermogravimetric analysis (TGA) comprising a thermogram substantially as shown in FIG. 8.

13. A hydrate of a compound of Formula IX-1a:

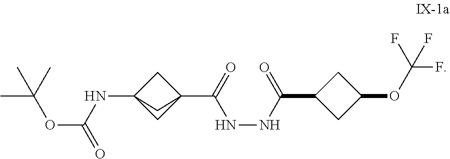

14. A dioxalate salt of 3-(5-((1s,3s)-3-(trifluoromethoxy)cyclobutyl)-1,3,4-oxadiazol-2-yl)bicyclo[1.1.1]pentan-1-amine having the following structure:

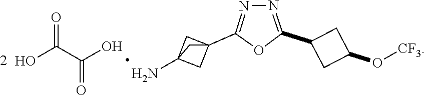

15. A 4-chlorophenoxyacetic acid salt of 3-(5-((1s,3s)-3-(trifluoromethoxy)cyclobutyl)-1,3,4-oxadiazol-2-yl)bicyclo[1.1.1]pentan-1-amine having the following structure:

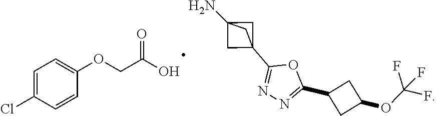

16. Crystalline 3-(5-((1s,3s)-3-(trifluoromethoxy)cyclobutyl)-1,3,4-oxadiazol-2-yl)bicyclo[1.1.1]pentan-1-amine (Compound XI-1a) free base Form A: 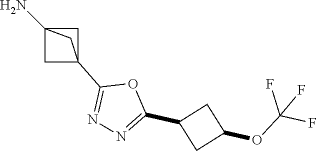
characterized by an X-ray powder diffraction pattern comprising one or more, or two, or three, or four, or five, or six, or seven peaks selected from 6.6, 10.9, 14.5, 19.9, 21.9, 25.0, 25.9° 2θ±0.2° 2θ, as determined on a diffractometer using Cu-Kα radiation (λ=1.54059 Å).

17. A compound selected from the group consisting of: 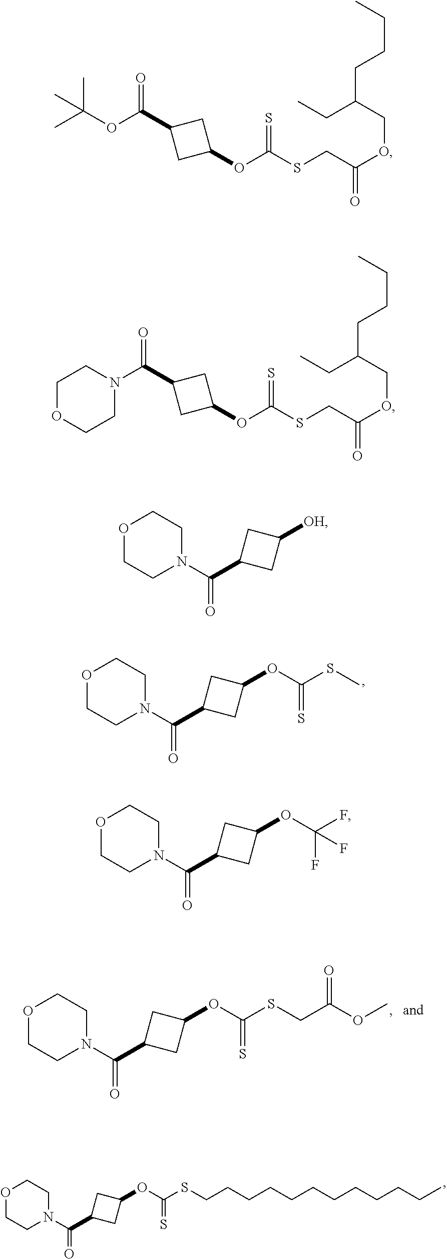
or a salt of each thereof.

18. A method of preparing a compound of Formula V-2: 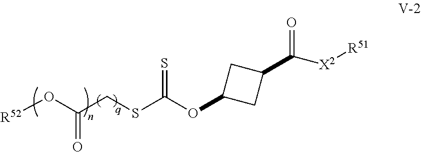
or a salt thereof, comprising contacting a compound of Formula IIa-1: 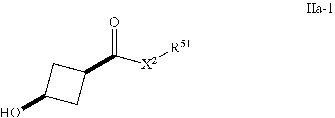
with an activating agent and then contacting with a compound of Formula IV: 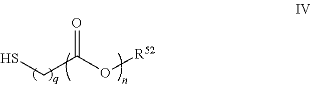
under reaction conditions sufficient to provide the compound of Formula V-2, wherein:
n is 0 or 1;
q is 1, 2, 3, 4, 5, 6, 7, 8, 9, 10, 11, or 12;
X² is O or NR⁵³;
R⁵¹ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino;
R⁵² is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; and
R⁵³ is hydrogen, C₁₋₁₂ alkyl, or silyl, wherein the C₁₋₁₂ alkyl or silyl is optionally substituted with one or more halo or C₁₋₁₂ alkyl independently optionally substituted by one or more oxo, halo, hydroxyl, or amino; or
R⁵¹ and R⁵³ are taken together with the nitrogen atom to which they are attached to form heterocyclyl independently optionally substituted by one or more halo or C₁₋₁₂ alkyl independently optionally substituted by one or more oxo, halo, hydroxyl, or amino;
provided the compound is not ethyl cis-3-[(methylthio)thioxomethoxy]cyclobutanecarboxylate.

19. A method of preparing a compound of Formula IX-2: 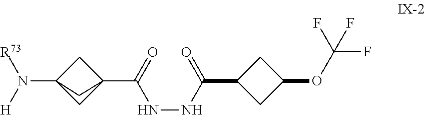
or a solvate thereof, comprising:
contacting a salt of a compound of Formula VIa-1a: 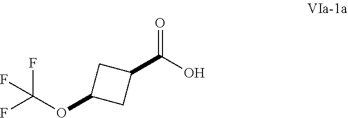 [SMILES: O=C(O)[C@H]1C[C@@H](OC(F)(F)F)C1]
with an acid under reaction conditions sufficient to provide the compound of Formula VIa-1a; and
contacting the compound of Formula VIa-1a with a compound of Formula VIIIa or a salt thereof: 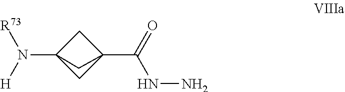
under conditions sufficient to provide the compound of Formula IX-2 or a solvate thereof,
wherein R⁷³ is hydrogen, a protecting group, or

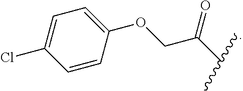 [SMILES: *C(=O)COc1ccc(Cl)cc1]

20. The method of claim 19, wherein R⁷³ is tert-butoxycarbonyl or of Formula

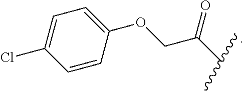 [SMILES: *C(=O)COc1ccc(Cl)cc1]

21. The method of claim 19 or 20, wherein the salt is Formula VII-2a: 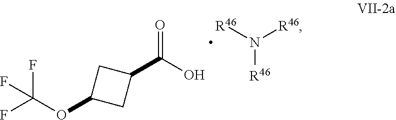
wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

22. A method of preparing a compound of Formula X-2: 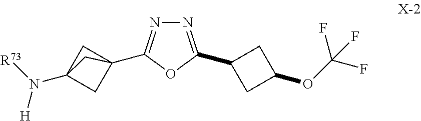
or a salt thereof, comprising:
contacting a salt of a compound of Formula VIa-1a: 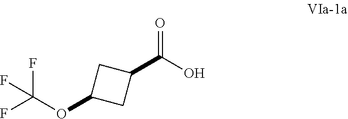
with a first acid under reaction conditions sufficient to provide the compound of Formula VIa-1a; and
contacting the compound of Formula VIa-1a with a compound of Formula VIIIa or a salt thereof: 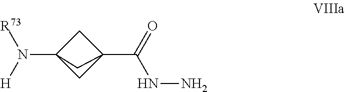
under conditions sufficient to provide a compound of Formula IX-2: 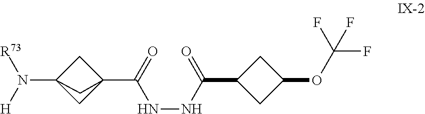
or a solvate thereof; and
contacting the compound of Formula IX-2 or a solvate thereof with a dehydrating agent and with a base under reaction conditions sufficient, to provide the compound of Formula X-2 or a salt thereof, wherein R⁷³ is hydrogen, a protecting group or

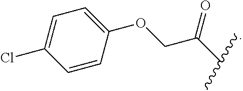 [SMILES: *C(=O)COc1ccc(Cl)cc1]

23. The method of claim 22, wherein the salt of the compound of Formula VIa-1a is Formula VII-2a: 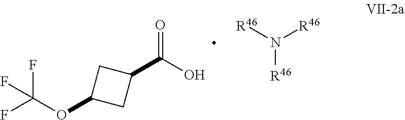
wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

24. The method of claim 22 or 23, wherein R⁷³ is tert-butoxycarbonyl or of Formula

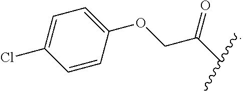 [SMILES: *C(=O)COc1ccc(Cl)cc1]

25. A method of preparing a compound of Formula I: 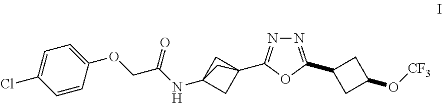
or a salt thereof, comprising:
contacting a salt of a compound of Formula VIa-1a: 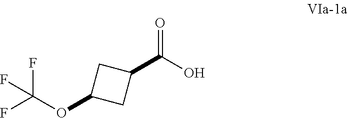 [SMILES: O=C(O)[C@H]1C[C@@H](OC(F)(F)F)C1]
with a first acid under reaction conditions sufficient to provide the compound of Formula VIa-1a; and
contacting the compound of Formula VIa-1a with a compound of Formula VIIIa or a salt thereof: 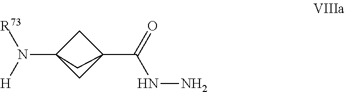
under conditions sufficient to provide a compound of Formula IX-2: 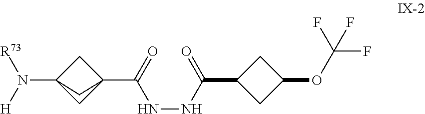
or a solvate thereof;
contacting the compound of Formula IX-2 or a solvate thereof with a dehydrating agent and with a base under reaction conditions sufficient, to provide a compound of Formula X-2: 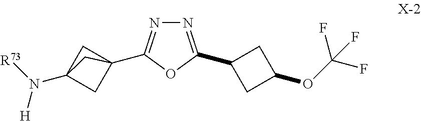
or a salt thereof; and
contacting the compound of Formula X-2 or a salt thereof, under deprotection conditions sufficient to provide the compound of Formula XI-1a: 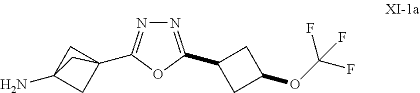
or a salt thereof, and
optionally contacting the compound of Formula XI-1a or a salt thereof with a compound of Formula XII-1: 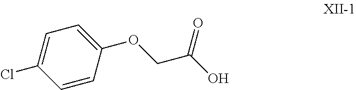 [SMILES: O=C(O)COc1ccc(Cl)cc1]
under reaction conditions sufficient to provide the compound of Formula I or a salt thereof, wherein R⁷³ is hydrogen, a protecting group, or

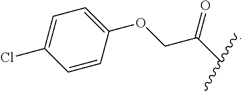 [SMILES: *C(=O)COc1ccc(Cl)cc1]

26. The method of claim 25, wherein the salt of the compound of Formula VIa-1a is Formula VII-2a: 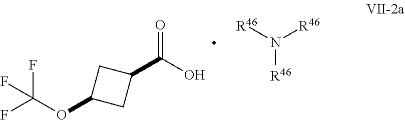
wherein the N(R⁴⁶)₃ moiety is selected from the group consisting of t-butylamine, L-lysine, piperazine, dicyclohexylamine, arginine, tromethamine, ethanolamine, dehydroabietylamine, and dibenzylamine.

27. The method of claim 25 or 26, wherein the salt of the compound of Formula X-2 is a hydrochloride salt, oxalic acid salt, and 4-chlorophenoxyacetic acid salt.

28. The method of any of claims 25-27, wherein the deprotection conditions comprises contacting the compound of Formula X-2 with a second acid.

29. The method of claim 25, wherein the reaction conditions providing the compound of Formula I comprises:
a) a temperature of about 0° C. to about 5° C., and warming to a temperature of about 20° C. to about 25° C.; and/or
b) optionally in presence of a base and diphenylphosphinic chloride.
